(19) United States
(12) Patent Application Publication
(10) Pub. No.: US 2019/0031743 A1
(43) Pub. Date: Jan. 31, 2019

(54) SCREENING METHODS FOR IDENTIFYING ANTIBODIES THAT BIND CELL SURFACE EPITOPES

(71) Applicant: ACHAOGEN, INC., South San Francisco, CA (US)

(72) Inventors: Lee Robert SWEM, Montara, CA (US); Ryan CIRZ, San Mateo, CA (US); Monica SCHWARTZ, South San Francisco, CA (US); Kristina BENDER, South San Francisco, CA (US); Shuang WU, South San Francisco, CA (US); Felix FINDEISEN, South San Francisco, CA (US); Malavika KANNUSWAMY, South San Francisco, CA (US); Dante RICCI, South San Francisco, CA (US); Ami PATEL, Foster City, CA (US)

(21) Appl. No.: 16/071,898

(22) PCT Filed: Jan. 27, 2017
(86) PCT No.: PCT/US2017/015515

§ 371 Date: Jul. 20, 2018

Related U.S. Application Data
(60) Provisional application No. 62/288,729, filed on Jan. 29, 2016.

(51) Int. Cl. C07K 16/12; C07K 16/00; G01N 33/569; G01N 33/543

(52) U.S. Cl. CPC C07K 16/1217; C07K 16/00; G01N 33/56911; C07K 2317/24; G01N 2500/10; G01N 2500/04; C07K 2317/21; G01N 33/5432

(57) ABSTRACT

Provided are assays or methods for identifying antibodies that bind to microorganisms, e.g., pathogenic microorganisms, such as bacteria other infectious agents. In some embodiments, the provided methods for identifying an antibody that binds the target microorganism involves gel encapsulation of antibody-producing cells in gel microdroplets with a target microorganism. Also provided are antibodies produced by the method. Also provided are antibodies that bind a conserved region or epitope across variants or species of Acenitobacter.

### CROSS-REFERENCE TO RELATED APPLICATIONS

This application claims priority from U.S. provisional application No. 62/288,729, filed Jan. 29, 2016, entitled “Screening Methods for Identifying Antibodies that Bind Cell Surface Epitopes,” the contents of which is incorporated by reference in its entirety.

### INCORPORATION BY REFERENCE OF SEQUENCE LISTING

The present application is being filed along with a Sequence Listing in electronic format. The Sequence Listing is provided as a file entitled 757832000140SeqList.TXT, created Jan. 27, 2017, which is 53,519 bytes in size. The information in the electronic format of the Sequence Listing is incorporated by reference in its entirety.

### FIELD

The present disclosure provides assays or methods for identifying antibodies that bind to microorganisms, e.g., pathogenic microorganisms, such as bacteria other infectious agents. In some embodiments, the methods for identifying an antibody that binds the target microorganism involves gel encapsulation of antibody-producing cells in gel microdroplets with a target microorganism. The present disclosure also provides antibodies produced by the method. The present disclosure also provides antibodies that bind a conserved region or epitope across variants or species of Acinetobacter.

### BACKGROUND

Multidrug-resistant bacteria have emerged worldwide and are increasing in prevalence, creating a substantial public health concern. The Centers for Disease Control and Prevention attributes at least 23,000 deaths in the U.S. each year to antibiotic-resistant infections, with some infection types associated with mortality rates as high as 50%. In difficult-to-treat Gram-negative pathogens, such as Acinetobacter spp. and Pseudomonas aeruginosa, rates of multi-drug resistance in the U.S. have been reported as 63% and 13%, respectively. The continued prevalence of these multidrug-resistant isolates has left clinicians with few treatment options for the patients with life-threatening infections. Addressing this urgent need for new antibiotics to treat multidrug-resistant Gram-negative infections is critical. There is a need in the art for methods of identifying therapeutics, e.g., antibodies, specific for pathogenic microorganisms, e.g. bacteria, that are resistant to many of the existing therapeutics. There also is a need in the art for methods of identifying therapeutics that are effective against a broad range of microorganisms, e.g., pathogens. Provided are methods and articles of manufacture that meets such need.

### SUMMARY

Provided herein are methods for identifying an antibody that binds a target microorganism, that includes the steps of: (a) obtaining a plurality of candidate antibody-producing cells; (b) encapsulating the plurality of candidate antibody-producing cells in gel microdroplets with a target microorganism; and (c) determining whether the antibody-producing cell(s) within the gel microdroplet produce an antibody that binds the target microorganism, thereby identifying an antibody that specifically binds to the target microorganism. In some embodiments, step (b) further includes encapsulating, in the microdroplets, an epitope-comprising fragment of the target microorganism or a variant thereof; and step (c) includes determining whether the antibody identified as binding the target microorganism also binds the epitope-comprising fragment thereof within the same gel microdroplet.

Provided herein are methods for identifying an antibody that binds a target microorganism, that includes the steps of: (a) obtaining a plurality of candidate antibody-producing cells; (b) encapsulating the plurality of candidate antibody-producing cells in gel microdroplets with a target microorganism and with an epitope-comprising fragment of the target microorganism or a variant thereof; and (c) determining whether the antibody-producing cell(s) within the gel microdroplet produce an antibody that binds the target microorganism and/or epitope-comprising fragment thereof present in the same gel microdroplet, thereby identifying an antibody that specifically binds to the target microorganism or epitope-comprising fragment thereof.

In some embodiments, the epitope-comprising fragment is bound to a solid support. In some embodiments, the solid support is a bead.

In some embodiments, the target microorganism is a bacterium, a fungus, a parasite or a virus. In some embodiments, the target microorganism is a bacterium or a fungus. In some embodiments, the microorganism is a multi-drug resistant microorganism.

In some embodiments, the microorganism is a bacterium that is a Gram-negative bacterium. In some embodiments, the Gram-negative bacterium is a proteobacterium. In some embodiments, the microorganism is a bacterium selected from among a species of Acinetobacter, Bdellovibrio, Burkholderia, Chlamydia, Enterobacter, Escherichia, Francisella, Haemophilus, Helicobacter, Klebsiella, Legionella, Moraxella, Neisseria, Pantoea, Pseudomonas, Salmonella, Shigella, Stenotrophomonas, Vibrio and Yersinia.

In some embodiments, the microorganism is selected from among Acinetobacter apis, Acinetobacter baumannii, Acinetobacter baylyi, Acinetobacter beijerinckii, Acinetobacter bereziniae, Acinetobacter bohemicus, Acinetobacter boissieri, Acinetobacter bouvetii, Acinetobacter brisouii, Acinetobacter calcoaceticus, Acinetobacter gandensis, Acinetobacter gerneri, Acinetobacter guangdongensis, Acinetobacter guillouiae, Acinetobacter gyllenbergii, Acinetobacter haemolyticus, Acinetobacter harbinensis, Acinetobacter indicus, Acinetobacter johnsonii, Acinetobacter junii, Acinetobacter kookii, Acinetobacter lwoffii, Acinetobacter nectaris, Acinetobacter nosocomialis, Acinetobacter pakistanensis, Acinetobacter parvus, Acinetobacter pitii, Acinetobacter pittii, Acinetobacter puyangensis, Acinetobacter qingfengensis, Acinetobacter radioresistans, Acinetobacter radioresistens, Acinetobacter rudis, Acinetobacter schindleri, Acinetobacter seifertii, Acinetobacter soli, Acinetobacter tandoii, Acinetobacter tjernbergiae, Acinetobacter towneri, Acinetobacter ursingii, Acinetobacter variabilis, Acinetobacter venetianus, Escherichia coli, Haemophilus influenzae, Klebsiella pneumoniae, Pseudomonas aeruginosa, Salmonella typhimurium, Shigella boydii, Shigella dysenteriae, Shigella flexneri, Shigella sonnei, Vibrio cholera and Yersinia pestis. In some embodiments, the microorganism is Acinetobacter baumannii.

In some embodiments, the microorganism is a bacterium that is a Gram-positive bacterium. In some embodiments, the microorganism is selected from among a species of Staphylococcus and Streptococcus.

In some embodiments, the microorganism is a fungus that is an Aspergillus species or a Candida species.

In some embodiments, the microorganism is a parasite that is a Coccidia or a Plasmodium species.

In some embodiments, the plurality of candidate antibody-producing cells are obtained from a donor that has been exposed to the target microorganism or an epitope-comprising fragment of the target microorganism or a variant thereof.

In some embodiments of the methods provided herein, the plurality of candidate antibody-producing cells is obtained by a method that includes the steps of: (i) expanding antibody-producing cells obtained from a donor that has been exposed to the target microorganism or an epitope-comprising fragment of the target microorganism or a variant thereof by introducing a cell composition containing the antibody-producing cells into an immunocompromised animal; and (ii) recovering the expanded antibody-producing cells, thereby obtaining the plurality of candidate antibody-producing cells.

In some embodiments, the cell composition containing the antibody-producing cells includes cells obtained from the spleen and/or lymph node of the donor. In some embodiments, the cell composition includes T cells. In some embodiments, the cell composition includes peripheral blood mononuclear cells (PBMCs) that includes the antibody-producing cells.

In some embodiments, the immunocompromised animal is a SCID mouse.

In some embodiments, the cell composition containing the antibody-producing cells is introduced into the immunocompromised animal intravenously or by transplant into the immunocompromised animal's spleen.

In some embodiments of the methods provided herein, the antibody-producing cells are from a donor exposed to a first variant of the target microorganism or epitope-comprising fragment thereof, and prior to introducing the cell composition containing the antibody-producing cells into the immunocompromised animal, the method includes mixing or incubating the antibody-producing cells with a second variant of the target microorganism or epitope-comprising fragment thereof, wherein the introduced cell composition includes the antibody-producing cells complexed with the second variant of the target microorganism or epitope-comprising fragment thereof.

In some embodiments, the epitope-comprising fragment includes an essential protein or fragment of an essential protein of the target microorganism.

In some embodiments, the epitope-comprising fragment includes a bacterial outer membrane (OM) protein, a membrane protein, an envelope proteins, a cell wall protein, a cell wall component, a surface lipid, a glycolipid, a lipopolysaccharide, a glycoprotein, a surface polysaccharide, a capsule, a surface appendage, a flagellum, a pilus, a monomolecular surface layer, or an S-layer or a fragment thereof derived from the target microorganism.

In some embodiments, the epitope-comprising fragment includes a lipid from the surface of the target microorganism. In some embodiments, the epitope-comprising fragment includes a lipopolysaccharide (LPS) or a lipoprotein.

In some embodiments, the epitope-comprising fragment includes an outer membrane (OM) protein. In some embodiments, the OM protein is selected from among BamA, LptD, AdeC, AdeK, BtuB, FadL, FecA, FepA, FhaC, FhuA, LamB, MepC, MexA, NalP, NmpC, NspA, NupA, Omp117, Omp121, Omp200, Omp71, OmpA, OmpC, OmpF, OmpG, OmpT, OmpW, OpcA, OprA, OprB, OprF, OprJ, OprM, OprN, OstA, PagL, PagP, PhoE, PldA, PorA, PorB, PorD, PorP, SmeC, SmeF, SrpC, SucY, TolC, TtgC and TtgF. In some embodiments, the OM protein is BamA or LptD.

In some embodiments, the epitope-comprising fragment is prepared by solubilization of the OM protein or a fragment thereof. In some embodiments, solubilization is carried out by addition of one or more detergent or surfactant.

In some embodiments of the methods provided herein, the method also includes refolding of the epitope-comprising fragment prior to mixing or incubating with the antibody-producing cells. In some embodiments, the refolding is carried out in the presence of one or more detergent or surfactant.

In some embodiments, the detergent or surfactant is selected from among lauryldimethylamine oxide (LDAO), 2-methyl-2,4-pentanediol (MPD), an amphipol, amphipol A8-35, C8E4, Triton X-100, octylglucoside, DM (n-Decyl-β-D-maltopyranoside), DDM (n-Dodecyl-β-D-maltopyranoside, 3-[(3-Cholamidopropyl)dimethylammonio]-1-propanesulfonate (CHAPS) and 3-[(3-cholamidopropyl)dimethylammonio]-2-hydroxy-1-propanesulfonate (CHAPSO).

In some embodiments of the methods provided herein, the method also includes replacing some or all of the detergent and/or surfactant in the preparation with an amphipathic polymer or a surfactant.

In some embodiments, prior to mixing or incubating with the antibody-producing cells, excess detergent or surfactant is removed or reduced from the preparation of the epitope-comprising fragment to a level or amount that is not toxic to and/or does not induce lysis of the antibody-producing cells.

In some embodiments, the first and second variant each independently includes an epitope-comprising fragment of the target microorganism. In some embodiments, the first and the second variant shares at least one conserved region or domain. In some embodiments, the first and the second variant each comprise at least one region or domain that differs from each other.

In some embodiments, the first and second variant includes an OM protein or fragment thereof derived from two different clinical isolates of the same microorganism.

In some embodiments, the first variant and/or second variant is a full-length OM protein and the other of the first and/or second variant is a fragment of the OM protein that includes deletion of an immunodominant epitope or loop of the OM protein.

In some embodiments, the identified antibody binds to the at least one conserved region or domain of the target microorganism.

In some embodiments of the methods provided herein, the donor has been immunized or infected with the target microorganism or an epitope-comprising fragment of the target microorganism or a variant thereof. In some embodiments, the donor is an immunized animal or an infected animal. In some embodiments, the donor is a mammal or a bird. In some embodiments, the donor is a human, a mouse or a chicken. In some embodiments, the donor is a human donor who was infected by the microorganism. In some embodiments, the donor is a genetically modified non-human animal that produces partially human or fully human antibodies.

In some embodiments of the methods provided herein, the antibody-producing cells comprise peripheral blood mononuclear cells (PBMCs), B cells, plasmablasts or plasma cells. In some embodiments, the antibody-producing cells comprise B cells, plasmablasts or plasma cells.

In some embodiments, the plurality of candidate antibody-producing cells are selected from the donor by a positive or negative selection to isolate or enrich for B cells. In some embodiments, the B cell is a plasmablast or a plasma cell. In some embodiments, the selection is a positive selection based on expression of a cell surface marker selected from among one or more of: CD2, CD3, CD4, CD14, CD15, CD16, CD34, CD56, CD61, CD138, CD235a (Glycophorin A) and FceRIa. In some embodiments, the antibody-producing cells comprise CD138+ cells. In some embodiments, at least or at least about 50%, 60%, 70%, 80%, 85%, 90%, 95%, or more of the cells are plasma cells or plasmablasts and/or are CD138+ cells.

In some embodiments, the antibody is an antibody or an antigen-binding fragment thereof.

In some embodiments, the gel microdroplet is generated by a microfluidics-based method. In some embodiments, the gel microdroplet includes material selected from among agarose, carrageenan, alginate, alginate-polylysine, collagen, cellulose, methylcellulose, gelatin, chitosan, extracellular matrix, dextran, starch, inulin, heparin, hyaluronan, fibrin, polyvinyl alcohol, poly(N-vinyl-2-pyrrolidone), polyethylene glycol, poly(hydroxyethyl methacrylate), acrylate polymers and sodium polyacrylate, polydimethyl siloxane, cis-polyisoprene, Puramatrix™, poly-divenylbenzene, polyurethane, or polyacrylamide or combinations thereof.

In some embodiments, the gel microdroplet includes agarose. In some embodiments, the agarose is low gelling temperature agarose. In some embodiments, the agarose has a gelling temperature of lower than about 35° C., about 30° C., about 25° C., about 20° C., about 15° C., about 10° C. or about 5° C. In some embodiments, the agarose has a gelling temperature of between about 5° C. and about 30° C., about 5° C. and about 20° C., about 5° C. and about 15° C., about 8° C. and about 17° C. or about 5° C. and about 10° C.

In some embodiments of the methods provided herein, step (b) also includes incubating the gel microdroplets at a temperature of between about 0° C. and about 5° C. for about 1 minute to about 10 minutes subsequent to encapsulation.

In some embodiments, the bead, such as the bead bound to the epitope-comprising fragment thereof, has an average diameter of between about 100 nm and about 100 μm, or between about 3 μm and about 5 μm.

In some embodiments, the average ratio of candidate antibody-producing cell per gel microdroplet is less than or less than about 1. In some embodiments, the average ratio of candidate antibody-producing cell per gel microdroplet is between about 0.05 and about 1.0, about 0.05 and about 0.5, about 0.05 and about 0.25, about 0.05 and about 0.1, about 0.1 and about 1.0, about 0.1 and about 0.5, about 0.1 and about 0.25, about 0.25 and about 1.0, about 0.25 and about 0.5 or 0.5 and about 1.0, each inclusive. In some embodiments, the average ratio of candidate antibody-producing cells per microdroplet is or is about 0.1.

In some embodiments, the average ratio of the microorganism per gel microdroplet is between about 50 and about 150 or about 50 and about 100.

In some embodiments, the average ratio of the bead per gel microdroplet is between about 2 and about 10 or about 3 and about 5.

In some embodiments, the average ratio of the candidate cell to microorganism to bead is about 0.1:100:10.

In some embodiments, the gel microdroplets comprise growth media and are surrounded by a non-aqueous environment. In some embodiments, the non-aqueous environment includes an oil. In some embodiments, the oil is gas permeable.

In some embodiments of the methods provided herein, the method also includes incubating the gel microdroplets at a temperature of at or about 37° C. prior to step (c). In some embodiments, the gel microdroplets are incubated in growth media.

In some embodiments of the methods provided herein, the method also includes, prior to step (c), introducing into the gel microdroplets a reagent that binds to antibodies, said reagent that includes a detectable moiety. In some embodiments, the reagent includes a secondary antibody specific for antibodies produced by the encapsulated antibody-producing cells.

In some embodiments, determining whether the antibody-producing cell(s) within the gel microdroplet produce an antibody that binds the target microorganism and/or epitope-comprising fragment thereof present in the same gel microdroplet includes detecting the presence of a complex that includes the steps of: (i) the target microorganism or epitope-comprising fragment thereof; (ii) the antibody produced by the antibody-producing cell; and (iii) the reagent that includes the detectable moiety bound, wherein the presence of the complex indicates that the antibody specifically binds the target microorganism or epitope-comprising fragment thereof.

In some embodiments, determining whether the antibody-producing cell(s) within the gel microdroplet produce an antibody that binds the target microorganism and/or epitope-comprising fragment thereof present in the same gel microdroplet that includes the step of determining whether the presence of the antibody modifies a phenotypic characteristic of the target microorganism in the same gel microdroplet, wherein the presence of the modified phenotypic characteristic indicates that the antibody specifically binds the target microorganism or epitope-comprising fragment thereof.

In some embodiments, the modified phenotypic characteristic is selected from among cell growth, cell death, changes in in behavior, binding, transcription, translation, expression, protein transport, cellular or membrane architecture, adhesion, motility, cellular stress, cell division and/or cell viability.

In some embodiments, determining whether the antibody-producing cell(s) within the gel microdroplet produce an antibody that binds the target microorganism and/or epitope-comprising fragment thereof present in the same gel microdroplet includes detecting a signal produced by a reporter molecule, wherein the signal is produced in the presence of the modified phenotypic characteristic. In some embodiments, the microorganism includes a polynucleotide encoding the reporter molecule. In some embodiments, the polynucleotide includes a regulatory region operably linked to a sequence encoding the reporter molecule, wherein the regulatory region is responsive to the modified phenotypic characteristic. In some embodiments, the regulatory region includes a promoter.

In some embodiments, the modified phenotypic characteristic includes cellular stress and the signal is produced in the presence of the cellular stress. In some embodiments, the cellular stress includes stress to the outer membrane (OM) of the bacterium. In some embodiments, the signal produced by the reporter molecule is detected with a detectable moiety.

In some embodiments, the signal produced by the reporter molecule includes a fluorescent signal, a luminescent signal, a colorimetric signal, a chemiluminescent signal or a radioactive signal. In some embodiments, the reporter molecule is a fluorescent protein, a luminescent protein, a chromoprotein or an enzyme.

In some embodiments, determining whether the antibody-producing cell(s) within the gel microdroplet produce an antibody that binds the target microorganism and/or epitope-comprising fragment thereof present in the same gel microdroplet includes determining whether the presence of the antibody kills the target microorganism in the same gel microdroplet, wherein killing of the target microorganism indicates that the antibody specifically binds the target microorganism or epitope-comprising fragment thereof. In some embodiments, the gel microdroplets comprise a detectable moiety indicative of cell death.

In some embodiments, the detectable moiety includes one or more detectable label selected from among a chromophore moiety, a fluorescent moiety, a phosphorescent moiety, a luminescent moiety, a light absorbing moiety, a radioactive moiety, and a transition metal isotope mass tag moiety.

In some embodiments of the methods provided herein, the method also includes the step of: (d) isolating the microdroplet that includes the cell producing the identified antibody or isolating polynucleotides encoding the antibody identified as specifically binding the target microorganism or epitope-comprising fragment thereof. In some embodiments, isolation is carried out using a micromanipulator or an automated sorter.

In some embodiments of the methods provided herein, the method also includes the step of: (e) determining the sequence of the nucleic acids encoding the identified antibody. In some embodiments, determining the sequence of the nucleic acids is carried out using nucleic acid amplification and/or sequencing. In some embodiments, determining the sequence of the nucleic acids is carried out using single cell PCR and nucleic acid sequencing.

In some embodiments of the methods provided herein, the method also includes the step of: (f) introducing a polynucleotide that contains a sequence of the nucleic acids encoding the identified antibody or fragment thereof into a cell.

In some embodiments, the provided method is completed within about 60 days, 50 days, 40 days, 30 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days or 1 day from completion of step (a).

In some embodiments, the provided method is completed within about 30 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days or 1 day from completion of step (a).

Also provided herein are antibodies identified using the methods provided herein, or any antigen-binding fragments of the antibody. In some embodiments, the provided antibodies bind to an epitope present in the at least one conserved region or domain of BamA (β-barrel assembly machinery) of a Gram-negative bacterium.

Also provided herein are antibodies or antigen-binding fragments thereof, wherein said antibody or antigen-binding fragment thereof binds to an epitope present in at least one conserved region or domain of BamA (β-barrel assembly machinery) of a Gram-negative bacterium.

In some embodiments of the provided antibodies or antigen-binding fragments thereof, the Gram negative bacterium is an Acinetobacter species. In some embodiments, the Gram negative bacterium is Acinetobacter baummannii. In some embodiments, the conserved region or domain is a conserved region or domain that is shared between BamA from A. baumannii ATCC 19606 and A. baumannii ATCC 17978. In some embodiments, the conserved region or domain includes amino acid residues 423-438, 440-460, 462-502, 504-533, 537-544, 547-555, 557-561, 599-604, 606-644, 646-652, 659-700, 702-707, 718-723, 735-747, 749-760, 784-794, 798-804, 806-815 and 817-841 A. baumannii BamA sequence set forth in SEQ ID NO:11. In some embodiments, the conserved region or domain includes the sequences set forth in SEQ ID NOS:12-20.

In some embodiments, the epitope is a contiguous or non-contiguous sequence of the conserved region or domain.

In some embodiments, the antibody or antigen-binding fragment is human.

In some embodiments, the antibody or antigen-binding fragment is a humanized antibody. In some embodiments, the antibody or antigen-binding fragment thereof is produced by antibody-producing cells from a transgenic animal engineered to produce humanized antibodies. In some embodiments, the antibody or antigen-binding fragment is recombinant. In some embodiments, the antibody or antigen-binding fragment is monoclonal.

In some embodiments, the provided antibodies or antigen-binding fragments thereof is an antigen-binding fragment.

In some embodiments, the provided antibodies or antigen-binding fragments thereof also includes an affinity tag, a detectable protein, a protease cleavage sequence, a linker or a nonproteinaceous moiety.

In some embodiments, the provided antibodies or antigen-binding fragments have an equilibrium dissociation constant (K_{D}) for A. baumannii BamA of at or less than or less than about 400 nM, 300 nM, 200 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM.

Also provided herein are polynucleotides encoding any of the antibodies or antigen-binding fragments thereof provided herein.

Also provided herein are compositions that contain any of the antibodies or antigen-binding fragments thereof provided herein. In some embodiments, the composition also contains a pharmaceutically acceptable excipient.

Also provided herein are compositions that contain a plurality of microdroplets, where each microdroplet contains: a candidate antibody-producing cell; and a target microorganism. In some embodiments, each microdroplet also contains the target microorganism or epitope-comprising fragment thereof or a variant thereof bound to a solid support. In some embodiments, the target microorganism contains a polynucleotide encoding a reporter molecule.

Also provided herein are libraries of gel microdroplets, where each microdroplet contains: a candidate antibody-producing cell; and a target microorganism. In some embodiments, each microdroplet also contains the target microorganism or epitope-comprising fragment thereof or a variant thereof bound to a solid support. In some embodiments, the target microorganism contains a polynucleotide encoding a reporter molecule.

### BRIEF DESCRIPTION OF THE DRAWINGS

FIG. 1 provides a diagram of an embodiment of the provided method, which includes, in some aspects, B cell enrichment from a source of antibody-expressing B cells, functional antibody selection and single cell cloning. In some instances, the provided methods can be termed rapid antibody discovery (RAD) platform.

FIG. 2 provides a schematic diagram of one embodiment of rare B cell enrichment in the RAD platform. This method allows the enrichment of antibodies to highly conserved epitopes on the target antigen of interest, by immunizing with one variant and enriching with a second variant that only has the conserved epitopes in common. The example target is shaded according to amino acid conservation. Light shading corresponds to variable regions and dark shading corresponds to conserved regions.

FIG. 3 demonstrates an embodiment of functional antibody selection. This embodiment of the Pathogen Antibody Trap (PAT) technology allows detection of antibody secreting cells that are producing rare antibodies. The green fluorescent signal (light gray spots with arrows) indicate that antibody binding can be seen on the beads and bacteria within the positive PATs.

FIG. 4 provides a homology model of BamA. The left panel shows a ribbon structure, while the right panel shows a space-fill model of A. baumannii BamA. The amino acids are labelled according to conservation among a panel of A. baumannii clinical isolates. Loop 4 is substantially diverse, but a highly conserved epitope is found on the extracellular surface. Light shading corresponds to variable regions and dark shading corresponds to conserved regions.

FIG. 5 shows a schematic of an embodiment of the rare B cell expansion step, exemplified with BamA variants. Each dark spot in the B cell IgG specific analysis represents a B cell that secretes an antibody. Each dark spot in the BamA-variant 2 specific analysis represents a B cell that producing an antibody to a conserved epitope.

FIGS. 6A-6B show immunofluorescence of functional antibody selection. Green fluorescence (indicated by light gray spots and arrows) depicts signal from goat anti-mouse-AlexaFluor488; Arrow depicts signal from Antibody-bound bacteria; Arrowhead depicts signal from Antibody-bound BamA-coated bead; Open arrow depicts signal from unlabeled bacteria; and open arrowhead depicts signal from unlabeled antigen-coated beads; scale bar=25 μm; FIG. 6A depicts a center particle containing a B cell secreting an antibody to a conserved surface-exposed BamA epitope, identified by fluorescent signal from both bacteria and beads. FIG. 6B depicts a single selected particle in pipette tip.

FIG. 7 shows binding of a recombinant antibody to a highly conserved epitope of BamA. Representative ELISA curve (duplicate samples). A recombinant antibody that was identified in the particle screen is shown to bind specifically to three BamA variants (variants 1, 3 and 4), but not a negative control protein (BSA), indicating the epitope is in a highly conserved region of BamA.

FIGS. 8-10 are diagrams showing various embodiments of gel-encapsulated screening methodologies employed in certain embodiments of the provided methods.

FIGS. 11A-11C show the detection of microdroplets that contain antibody-producing cells with bacterial cells with a reporter responsive to outer membrane (OM) stress. Fluorescence signal indicates the presence of disruption of the OM and/or OM stress.

FIG. 12 shows a histogram of optical density (OD) measurements from an ELISA binding assay of nine hybridoma-generated antibodies that target LptD/LptE. The ELISA was performed to assess binding against LptD/LptE at 1:50 and 1:250 dilution, and against a negative control antigen (BamA) at 1:50.

FIGS. 13A and 13B show histogram overlay of fluorescence signal of cell binding response of polyclonal sera generated from mice immunized with a BamA variant 1 to A. baumannii strains differentially expressing BamA variant 5. FIG. 13A shows the binding A. baumannii that does not express BamA on the surface. FIG. 13B shows the binding to A. baumannii expressing BamA variant 5.

### DETAILED DESCRIPTION

Provided herein are assays or methods for identifying antibodies that bind to microorganisms, e.g., pathogenic microorganisms such as bacteria other infectious agents. In some embodiments of the methods provided herein, the method includes identifying an antibody that binds a target microorganism. In some embodiments, the method involves the steps of (a) obtaining a plurality of candidate antibody-producing cells; (b) encapsulating the plurality of candidate antibody-producing cells in gel microdroplets with a target microorganism; and (c) determining whether the antibody-producing cell(s) within the gel microdroplet produce an antibody that binds the target microorganism, thereby identifying an antibody that specifically binds to the target microorganism. In particular embodiments, the antibodies are capable of inhibiting the growth or proliferation of the target cells, bacteria and other infectious agents. In particular embodiments, the antibodies kill the target cells, bacteria and other infectious agents.

Therapeutic antibodies have many advantages over traditional small molecule drugs, making them an attractive option for the treatment of emerging infectious diseases. Antibodies have exquisite specificity for target antigen, which greatly reduces the risk of off-target toxicity. This beneficial safety profile allows prophylactic and therapeutic treatment options, and a margin of safety appropriate for pediatric and elderly populations, which are often at highest risk during emerging infectious disease outbreaks. Additionally, most human antibodies have a long half-life (˜21 days) with predictable human clearance, which could enable single-dose treatment options in infected individuals and further enable prophylactic treatment options in high risk individuals. These favorable antibody properties also support a rapid clinical development path essential for swift response during infectious disease outbreaks. Not only is clinical development expedited, but new antibody discovery technologies make therapeutic antibody identification faster than traditional small molecule discovery. Finally, it is well established that drug combinations limit resistance, but small molecule drug combinations are difficult to rapidly develop because of potential drug-drug interactions and unanticipated off-target toxicities. Antibodies offer the possibility of quickly formulating antibody cocktails that would limit resistance and increase the breadth of potency. For the above reasons, human or humanized antibodies are useful for the treatment of infectious diseases.

Traditionally, it has been difficult to identify single antibodies that can broadly neutralize all clinical isolates of a given pathogen. This is because pathogens are in an “arms race” with the host immune response. For example, when the host immune response is dominated by functional neutralizing antibodies, the pathogen must escape the host defense to remain a successful pathogen.

Pathogens use two fundamental methods to keep the immuno-dominant antibody response from being broadly neutralizing. First, they produce highly variable and immuno-dominant epitopes on essential proteins, tricking the host to produce large numbers of non-functional antibodies toward highly variable epitopes. These epitopes act as decoys that shift the focus of the host immune response away from more conserved important epitopes. Second, they protect the conserved functional epitopes by making them not easily accessible, thereby greatly reducing the number of antibodies that bind to these important epitopes. This makes the frequency of broadly neutralizing antibodies quite low and nearly impossible to discover using traditional antibody discovery methods, such as hybridoma. Recent examples of this paradigm can be found in the literature relating to the discovery of broadly neutralizing influenza A antibodies. The majority of antibodies raised after immunization or during an active influenza infection bind to highly variable epitopes on the influenza A surface. Therefore, the antibody response is not protective during the subsequent season, allowing individuals to become infected with influenza many times throughout their life. A highly conserved epitope on the surface of influenza was identified decades ago, but it wasn't until recent advances in immunology and molecular biology that allowed the discovery of antibodies that could bind this epitope and broadly neutralize all influenza A.

The provided methods provide an efficient and effective method to rapidly generate, screen and identify candidate antibody-producing cells of interest that specifically bind to an epitope-comprising fragment of interest, such as an epitope that is conserved across variants and/or species of target microorganisms.

### I. Definitions

Unless defined otherwise, all technical and scientific terms used herein have the same meaning as commonly understood by those of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the term “effective amount” refers to at least an amount effective, at dosages and for periods of time necessary, to achieve the desired result, e.g., an enhanced immune response to an antigen, a decrease in tumor growth or metastasis, or a reduction in tumor size. An effective amount can be provided in one or more administrations.

As used herein, the singular form “a”, “an”, and “the” includes plural references unless indicated otherwise.

Reference to “about” a value or parameter herein refers to the usual error range for the respective value readily known to the skilled person in this technical field. In particular embodiments, reference to about refers to a range within 10% higher or lower than the value or parameter, while in other embodiments, it refers to a range within 5% or 20% higher or lower than the value or parameter. Reference to “about” a value or parameter herein includes (and describes) aspects that are directed to that value or parameter per se. For example, description referring to “about X” includes description of “X.”

As used herein, the term “modulating” means changing, and includes positive modulating, such as “increasing,” “enhancing,” “inducing” or “stimulating,” as well as negative modulating such as “decreasing,” “inhibiting” or “reducing,” typically in a statistically significant or a physiologically significant amount as compared to a control. An “increased,” “stimulated” or “enhanced” amount is typically a “statistically significant” amount, and may include an increase that is 1.1, 1.2, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the amount produced by no treatment as described herein or by a control treatment, including all integers in between. A “decreased,” “inhibited” or “reduced” amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%), 80%), 85%, 90%), 95%, or 100% decrease in the amount produced by no treatment as described herein or by a control treatment, including all integers in between.

By “statistically significant,” it is meant that the result was unlikely to have occurred by chance. Statistical significance can be determined by any method known in the art.

Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

It is understood that aspects and embodiments of the invention described herein include “comprising,” “consisting,” and “consisting essentially of” aspects and embodiments.

The terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like. Also encompassed by the term are Fab′, Fv, F(ab′)₂, and or other antibody fragments that retain specific binding to antigen, and monoclonal antibodies. Antibodies may exist in a variety of other forms including, for example, Fv, Fab, and (Fab′)₂, as well as bi-functional (i.e., bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988)). (See, generally, Hood et al., “Immunology”, Benjamin, N.Y., 2nd ed. (1984), and Hunkapiller and Hood, Nature, 323, 15-16 (1986)). Also encompassed are polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, heavy chain variable (V_{H}) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof also referred to herein as “antigen-binding fragments.” The term also encompasses intact or full-length antibodies, including antibodies of any class or sub-class, including IgG and sub-classes thereof, IgM, IgE, IgA, and IgD.

As used herein, vector (or plasmid) refers to a nucleic acid construct, typically a circular DNA vector, that contains discrete elements that are used to introduce heterologous nucleic acid into cells for either expression of the nucleic acid or replication thereof. The vectors typically remain episomal, but can be designed to effect stable integration of a gene or portion thereof into a chromosome of the genome. In some cases, vectors contain an origin of replication that allows many copies of the plasmid to be produced in a bacterial or eukaryotic cell without integration of the plasmid into the host cell DNA. Selection and use of such vectors are well known to those of skill in the art.

The terms “polynucleotide” and “nucleic acid molecule” are used interchangeably to refer to a single-stranded and/or double-stranded polynucleotides, such as deoxyribonucleic acid (DNA) and ribonucleic acid (RNA), as well as analogs or derivatives of either RNA or DNA. The length of a polynucleotide molecule is given herein in terms of nucleotides (abbreviated “nt”) or base pairs (abbreviated “bp”). Also included in the term “nucleic acid” are analogs of nucleic acids such as peptide nucleic acid (PNA), phosphorothioate DNA, and other such analogs and derivatives. Nucleic acids can encode gene products, such as, for example, polypeptides, regulatory RNAs, microRNAs, siRNAs and functional RNAs. Hence, nucleic acid molecule is meant to include all types and sizes of DNA molecules including cDNA, plasmids or vectors and DNA including modified nucleotides and nucleotide analogs.

The terms “polypeptide” and “protein” are used interchangeably to refer to a polymer of amino acid residues, and are not limited to a minimum length. Polypeptides may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

As used herein, ‘regulatory sequence’ or ‘regulatory region’ as used in reference to a specific gene, refers to the coding or non-coding nucleic acid control sequence within that gene that are necessary or sufficient to provide for the regulated expression of the coding region of a gene. Thus, the term encompasses promoter sequences, regulatory protein binding sites, upstream activator sequences and the like. Specific nucleotides within a regulatory region may serve multiple functions. For example, a specific nucleotide may be part of a promoter and participate in the binding of a transcriptional activator protein.

By “operably linked” is meant a functional linkage between a nucleic acid expression control sequence (such as a promoter) and a second nucleic acid sequence, wherein the expression control sequence directs transcription of the nucleic acid corresponding to the second sequence.

Percent “identical” or “identity” in the context of two or more nucleic acid or polypeptide sequences refers to two or more sequences that are the same or have a specified percentage of nucleic acid residues or amino acid residues, respectively, that are the same, when compared and aligned for maximum similarity, as determined using a sequence comparison algorithm or by visual inspection. “Percent sequence identity” or “% identity” or “% sequence identity or “% amino acid sequence identity” of a subject amino acid sequence to a reference amino acid sequence means that the subject amino acid sequence is identical (i.e., on an amino acid-by-amino acid basis) by a specified percentage to the reference amino acid sequence over a comparison length when the sequences are optimally aligned. Thus, 80% amino acid sequence identity or 80% identity with respect to two amino acid sequences means that 80% of the amino acid residues in two optimally aligned amino acid sequences are identical.

As used herein, the terms “engineered” and “recombinant” cells or “recombinant” nucleic acid molecules are intended to refer to a cell into which an exogenous DNA segment or gene, such as a cDNA or gene encoding at least one fusion protein has been introduced, or such nucleic acid molecules containing exogenous DNA segments or genes. Therefore, engineered cells are distinguishable from naturally occurring cells which do not contain a recombinantly introduced exogenous DNA segment or gene. Engineered cells are thus cells having a gene or genes introduced through human intervention. Recombinant cells include those having an introduced cDNA or genomic gene, and also include genes positioned adjacent to a promoter not naturally associated with the particular introduced gene.

As used herein, a “reporter molecule” refers to a molecule that is directly or indirectly detectable or whose presence is otherwise capable of being measured. In some aspects, receptor molecules include proteins that can emit a detectable signal such as a fluorescence signal, and enzymes that can catalyze a detectable reaction or catalyze formation of a detectable product. Reporter molecules also can include detectable nucleic acids. In some embodiments, a reporter molecule is a polypeptide which can be detected when it is expressed in the cell. In some cases, expression of the detectable reporter may lead to the production of a signal, for example a fluorescent, bio luminescent or colorimetric signal, which can be detected using routine techniques. The signal may be produced directly from the reporter, after expression, or indirectly through a secondary molecule, such as a labelled antibody.

The terms “reporter cell” and “reporter microorganism” are used interchangeably to refer to an engineered microorganism into which an exogenous or heterologous polynucleotide, such as a cDNA or gene, encoding a reporter molecule has been introduced. Therefore, reporter cells are distinguishable from naturally occurring microorganisms which do not contain a recombinantly introduced exogenous polynucleotide. Reporter cells are thus cells having a gene or genes introduced through human intervention and that express an exogenous reporter molecule.

As used herein, heterologous with reference to a polynucleotide or gene (also referred to as exogenous or foreign) refers to a nucleotide sequence that is not native to the organism or a gene contained therein or not normally produced in vivo by an organism, such as bacteria, from which it is expressed.

As used herein, a kit is a packaged combination that optionally includes other elements, such as additional reagents and instructions for use of the combination or elements thereof. Kits optionally include instructions for use.

The practice of the present disclosure will employ, unless otherwise indicated, conventional techniques of cell culturing, molecular biology (including recombinant techniques), microbiology, cell biology, biochemistry and immunology, which are within the skill of the art. Such techniques are explained fully in the literature, such as, Molecular Cloning: A Laboratory Manual, third edition (Sambrook et al., 2001) Cold Spring Harbor Press; Oligonucleotide Synthesis (P. Herdewijn, ed., 2004); Animal Cell Culture (R. I. Freshney), ed., 1987); Methods in Enzymology (Academic Press, Inc.); Handbook of Experimental Immunology (D. M. Weir & C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Manual of Clinical Laboratory Immunology (B. Detrick, N. R. Rose, and J. D. Folds eds., 2006); Immunochemical Protocols (J. Pound, ed., 2003); Lab Manual in Biochemistry: Immunology and Biotechnology (A. Nigam and A. Ayyagari, eds. 2007); Immunology Methods Manual: The Comprehensive Sourcebook of Techniques (Ivan Lefkovits, ed., 1996); Using Antibodies: A Laboratory Manual (E. Harlow and D. Lane, eds., 1988); and others.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### II. Method of Antibody Screening Using Pathogen Antibody Trap Technology (PAT)

Provided herein are methods to rapidly and effectively screen antibody-producing cells to identify an antibody that binds a target microorganism. The provided methods utilize gel encapsulation of antibody-producing cells, e.g., B cells and/or plasmablasts, and target microorganisms and/or antigens (e.g., in a gel microenvironment) to rapidly screen antibody producing B cells and/or plasmablasts for their ability to produce an antibody that has target microorganism-, e.g., bacterial- or fungal-cell binding, behavior modifying, or cidal activity. The provided methods are particularly useful for identifying single antibodies that can broadly neutralize all or a majority of clinical isolates of a given pathogenic microorganism, and for identifying antibodies that are effective in treating infections that are difficult to treat with conventional therapeutics, e.g., multidrug resistant microorganisms.

The present disclosure relates, in part, to methods and/or assays for identifying antibodies that bind to the surface of cells, bacteria and other infectious agents, e.g., microorganisms. In particular embodiments, the antibodies are capable of inhibiting the growth or proliferation of the target cells, bacteria and other infectious agents. In particular embodiments, the antibodies kill the target cells, bacteria and other infectious agents.

In some embodiments, the provided methods can identify antibodies that are difficult to identify using conventional methods, and/or can identify antibodies that target epitopes that are difficult to raise antibodies against. For example, in some embodiments, the provided methods can identify antibodies against targets that are essential and are conserved across many variants and/or species of target microorganisms, but which antibodies are difficult to identify or obtain. In some cases, the difficulties of identification is due to obstruction of the conserved and essential epitope in conventional methods of producing antibodies or antibody-producing cells, or the predominance of antibodies against variable, immunodominant epitopes of target microorganisms in conventional methods of producing antibodies or antibody-producing cells. In some embodiments, the provided methods allow efficient generation and/or screening of candidate antibody-producing cells that produce antibodies against desired target microorganisms and/or epitope-comprising fragment thereof, thereby reducing the time required for identification of specific antibodies of interest.

Existing methods for identifying technologies have numerous limitations. Currently, B cells are subjected to hybridoma fusion technology upstream of any binding or functional data regarding the antibody produced by that B cell. The hybridoma fusion technology is incredibly inefficient, with fusion rates of approximately 1 in every 5000 B cells. Therefore, the majority of the antibody repertoire produced by an immune response is not interrogated for or tested for antibody binding or function. Additionally, B cell hybridoma fusion partners are not readily available for species other than mouse, which limits the search for rare bacterial or fungal cell-binding and functional antibodies to a single donor animal species.

The provided methods do not rely on hybridoma technology and therefore the entire immune repertoire can be investigated for antibodies that bind or cause a modification of a phenotype on a microorganism, e.g., bacterial or fungal cell. This allows for the discovery of exceedingly rare antibodies, which is not feasible with hybridoma technology. In addition, the provided methods allow one to screen B cells and/or plasmablasts from any animal source that you desire, including but not limited to human, rat, chicken, llama, or, camel.

In particular embodiments of the provided methods, the antibody selection step uses an approach called Pathogen Antibody Trap (PAT) technology, based on gel encapsulation, to screen the antibodies being produced by single antibody-secreting B cells and/or plasmablasts, including enriched single B cells. In particular embodiments, the provided methods allow selection of only those B cells with the highest likelihood of producing functional antibodies prior to performing the more labor intensive steps of antibody cloning, production, and characterization.

In certain embodiments, antibody-producing cells, e.g., B cells are screened using gel encapsulation, e.g., PAT technology. In some embodiments, the PAT technology is typified by encapsulating single antibody secreting B cells and/or plasmablasts within small agarose microdroplets. In certain embodiments, PAT microdroplets are homogenous in size.

The provided methods for identifying an antibody that binds a target microorganism involves using gel microencapsulation of a plurality of candidate producing cells and particular target microorganisms, e.g., pathogenic microorganisms, or epitope-comprising fragment thereof, e.g., an antigen. In some embodiments, the methods include steps of: obtaining a plurality of candidate antibody-producing cells; encapsulating the plurality of candidate antibody-producing cells in gel microdroplets with a target microorganism; and determining whether the antibody-producing cell(s) within the gel microdroplet produce an antibody that binds the target microorganism, thereby identifying an antibody that specifically binds to the target microorganism.

Any microorganism, e.g., pathogen, e.g. any bacterial or fungal species, can be encapsulated within the gel microenvironment and subjected to the screening protocols in accord with the provided methods. Exemplary pathogens are described herein.

In particular embodiments, the technology of the present invention may be used to identify antibodies that bind to cell surface exposed proteins, carbohydrates, lipid moieties, or any combination thereof.

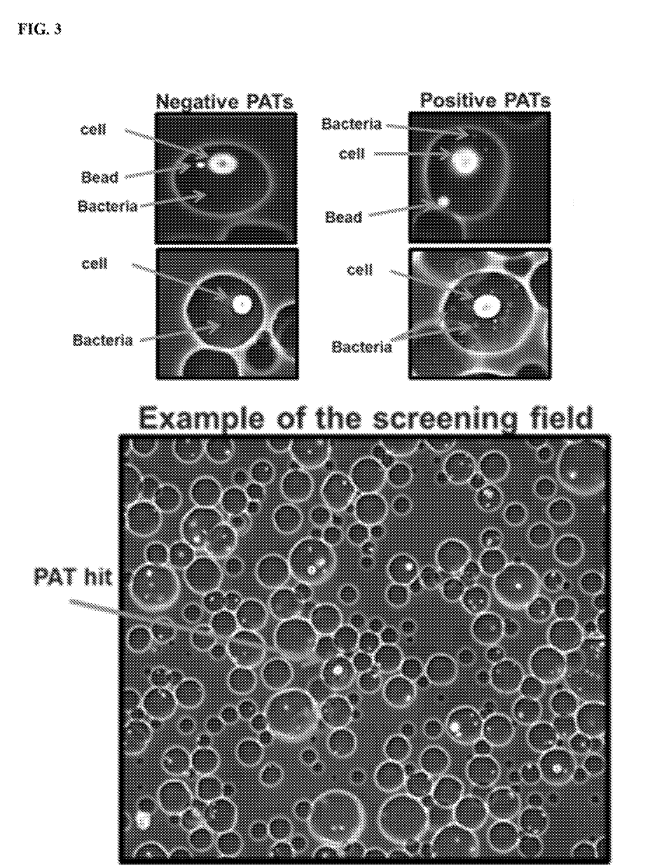

In some cases, beads conjugated to the immunoprotective protein of interest, e.g., epitope-comprising fragment from a target microorganism, can be co-encapsulated within agarose microdroplets. It is found herein that the provided methods can be carried out using microorganisms, e.g., pathogens, such as bacterial cells, which can be co-encapsulated within the same agarose microdroplet resulting in PAT encapsulation. As shown in FIG. 3, it is possible to encapsulate an antibody-producing cell, e.g., hybridoma cell and either one or both of an antigen-conjugated bead (e.g., beads conjugated with an epitope-comprising fragment of a target microorganism) and a microorganism (e.g., bacterial cell) to identify hybridoma cells that secrete an antibody that bind to the antigen (e.g. BamA) on the bead or on the bacterial surface. Using the provided methods, antigen-binding clones can be readily detected even after mixing at very low frequency with hybridoma cells producing antibodies that do not bind the antigen.

Thus, in some embodiments, the methods further include a step of encapsulating an epitope-comprising fragment of the target microorganism or a variant thereof in the microdroplets; and determining whether the antibody-producing cells identified as binding the target microorganism also binds the epitope-comprising fragment thereof within the same gel microdroplet.

In certain embodiments, the presence of a desired antibody is determined visually, e.g., by fluorescence microscopy. In some embodiments, the PATs are stained with a fluorescent secondary antibody to visualize and determine if the primary antibody binds with specificity to the target protein. Using low magnification fluorescent microscopy, punctuate fluorescent spots can be seen within the PAT if the secreted antibody binds to a recognized antigen. In some embodiments, binding of a secreted antibody is detected to an immunoprotective protein conjugated to the bead and to the target protein on the surface of the pathogen. In some such cases, antibody binding to the antigen conjugated bead (e.g., beads conjugated with an epitope-comprising fragment of a target microorganism) and to the pathogen surface, gives very high confidence that the antibody is target specific and able to recognize the naturally occurring, immunoprotective protein on the surface of the pathogen.

In particular embodiments, the PAT technology is used to functionally screen antibodies to directly identify antibodies that inhibit the target, e.g., inhibit the growth or proliferation of a target microorganism.

In some aspects, the PAT that contains the positive B cell of interest can be simply selected for cloning in the next phase of the discovery platform. Because the human eye can so rapidly discern a fluorescent signal within a positive PAT from the lack of signal in the negatives PATs, a single scientist can quickly screen hundreds of thousands of B cells using the PAT technology. Interestingly, the PAT method is much more efficient than fluidic separation systems such as FACS, which permits screening significantly more B cells to find rare antibodies of interest.

In some embodiments, the provided methods may be practiced using high throughput screening of thousand to millions or more gel-encapsulated antibody-producing cells. In some embodiments, millions of B cells can be PAT encapsulated and screened during a single discovery experiment using the provided methods. In certain embodiments, after encapsulation, the B cells are allowed to secrete antibody for a few hours within the agarose droplet before the antibodies are screened and selected. Embodiments of the methods described herein can be used to rapidly screen millions of antibody secreting B cells for pathogen, e.g. bacterial or fungal, cell binding or functional antibodies.

In some embodiments, at least 1 million B cells may be screened per day. In certain embodiments, the methods allow cloning of ˜100 antibodies per PAT screen. In certain embodiments, the methods enable transfection, purification, in vitro potency analysis of ˜100 antibodies per PAT screen.

Particular embodiments of the present disclosure are directed to a state-of-the-art antibody discovery platform that integrates rare B cell enrichment, functional antibody selection, followed by single B cell cloning, which can be termed the Rapid Antibody Discovery (RAD) platform. This platform allows the rapid expansion, selection, and discovery of large panels of functional human antibodies that bind to the most highly conserved and important target protein epitopes.

In certain embodiments, the present disclosure includes a method for identifying an antibody that specifically binds to a target microorganism, e.g., pathogen or epitope-comprising fragment thereof, comprising: (a) expanding antibody-producing cells obtained from an animal infected by or immunized with the target pathogen or epitope-comprising fragment thereof by introducing the antibody-producing cells into an immunocompromised animal; (b) encapsulating antibody-producing cells obtained from the immunocompromised animal following step (a) in gel microdroplets together with the target pathogen and/or epitope-comprising fragment thereof, wherein a plurality of the gel microdroplets comprise only one antibody-producing cell; and (c) determining whether the antibody-producing cell(s) within the gel microdroplet produce an antibody that binds the target pathogen and/or epitope-comprising fragment thereof present in the same gel microdroplet, thereby identifying an antibody that specifically binds to the target pathogen or epitope-comprising fragment thereof.

In some embodiments of the methods provided herein, the methods include a step for in vivo enrichment of or expansion of rare antibody-producing cells that produce antibodies against a specific target microorganism or an antigen or an epitope of the target microorganism. For example, in some embodiments, the plurality of candidate antibody-producing cells is obtained by a method that includes: (i) expanding antibody-producing cells obtained from a donor that has been exposed to the target microorganism or an epitope-comprising fragment of the target microorganism or a variant thereof by introducing antibody-producing cells into an immunocompromised animal; and (ii) recovering the expanded antibody-producing cells, thereby obtaining the plurality of candidate antibody-producing cells. In some embodiments, such steps can be used to enrich or expand rare antibody-producing cells of interest.

In certain embodiments, particular embodiments of the provided methods, e.g., the methods for screening antibody-producing cells, comprises one or more of the following steps: generation of B cells and/or plasmablasts producing humanized or human antibodies against a target of interest; 2) expansion of the B cells and/or plasmablasts, e.g., rare B cells, e.g., using in vivo enrichment, e.g., SCID expansion, to obtain cells enriched for desirable antibodies; 3) gel encapsulation methodologies for encapsulating single B cells with antigen and the pathogen of interest to select B cells of highest potential; and single B cell cloning. In certain embodiments, the human eye can be more adept than automated systems such as FACS at identifying the signal in the provided methods for screening antibody-producing cells. Thus, in certain embodiments, fluorescence microscopy is employed to rapidly identify and select the gel microdroplets containing cells of interest, e.g., cells producing the antibody that specifically binds to a target microorganism.

The present methods provide a platform that allows enrichment for the antibodies of highest therapeutic potential prior to engaging in the more labor intensive downstream steps of antibody discovery. Thus, in particular embodiments, the rare B cell enrichment phase allows for quickly generating large panels of antibody-producing cells, e.g., B cells and/or plasmablasts, with the desired functional activity and greatly improves the chances of successfully generating therapeutic antibody candidates and effective therapeutic antibodies.

In particular embodiments, the provided methods for screening antibody-producing cells, e.g., Rapid Antibody Discovery (RAD) platform, is used for the discovery of therapeutic antibodies for the treatment of infectious diseases. Bactericidal antibodies to target the most difficult to treat infectious diseases caused by Pseudomonas aeruginosa and Acinetobacter baumannii may be generated according to the provided methods. The provided methods can be used to rapidly generate and identify effective antibodies against microorganisms involved in infections that are difficult to treat by conventional therapies.

In certain embodiments, the provided methods are used to generate high affinity human antibodies that kill bacteria directly by binding to highly conserved epitopes on the essential outer membrane proteins BamA and LptD. Initial experiments described herein has shown experimental evidence and validation of these exemplary target antigens as being accessible to antibody binding and essential for bacterial fitness and survival.

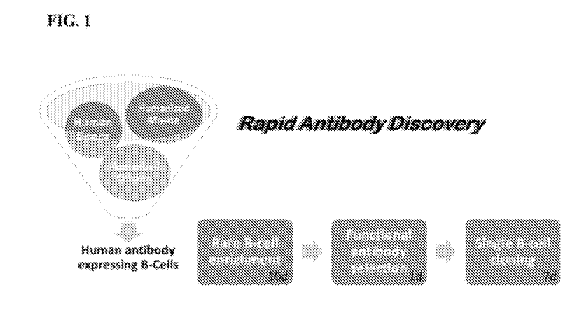

Although the platform is suitable for the discovery of antibodies against any target, it is particularly well-suited to rapidly respond to infectious diseases that pose significant threat to human health. In some embodiments, the provided methods can identify particular antibodies in a substantially shorter time than conventional methods of identifying antibodies. For example, in some embodiments, the method is completed within about 60 days, 50 days, 40 days, 30 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days or 1 day from obtaining the candidate antibody-producing cells. In some embodiments, the method is completed within about 30 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days or 1 day from obtaining the candidate antibody-producing cells. Specifically, the particular embodiments of the provided methods, e.g., antibody discovery platform technology can be broken into three phases: rare B cell enrichment (e.g., about 10 days), functional antibody selection (e.g., about 1 day), and single B cell cloning (e.g., about 7 days), which in-total would take approximately 18 days from B cell extraction to identification of therapeutic antibody candidates (e.g., see FIG. 1).

In some embodiments, the provided methods include identifying an antibody that specifically binds to the target microorganism. In some embodiments, the methods further include isolating the microdroplet comprising the cell producing the identified antibody or isolating polynucleotides encoding the antibody identified as specifically binding the target microorganism or epitope-comprising fragment thereof. In some embodiments, the methods further include determining the sequence of the nucleic acids encoding the identified antibody. In some embodiments, the isolation of antibody-producing cells that produce the antibody of interest and determination of sequences encoding the antibody of interest can be performed using nucleic acid amplification and/or sequencing methods. For example, in some embodiments, single cell PCR and cloning is used for isolation and sequence determination. In certain embodiments, the single B cell cloning phase of the methods for screening antibody-producing cells utilizes the ability of the provided methods to efficiently PCR amplify the heavy and light chain genes that encode the antibody produced within the selected gel microdroplet. In particular embodiments, PCR is performed at the single cell level, circumventing the requirement of 7-day B cell propagation step prior to PCR. Additionally, single cell PCR eliminates the need for a hybridoma fusion partner, which makes antibody discovery possible from any animal B cell source, including humans. Within just a few hours, the provided methods allow progression from a pool of enriched B cells and/or plasmablasts, to selecting the B cells and/or plasmablasts of greatest potential, and to begin PCR amplification of the nucleic acids that encode those antibodies.

The provided methods for screening antibody-producing cells offer many advantages over traditional antibody discovery platforms. First, it allows for the discovery of naturally occurring fully human antibodies, therefore eliminating, in some cases, the need for humanization and ultimately speeding up development timelines. Second, these methods allow for the expansion and enrichment of B cells and/or plasmablasts that produce antibodies to the most important epitopes of the microorganism, e.g., the immunoprotective proteins of interest, e.g., antigen or epitope of interest. Third, the gel encapsulation technology allows testing for antigen specificity and binding prior to committing valuable time and resources to cloning the genes that encode the antibody. In addition, the single B cell cloning technology coupled with linear DNA transfection technology significant reduces the time required compared to traditional antibody discovery methods. Therefore, in about 18 days, the provided methods for screening antibody-producing cells can generate a panel of fully human antibodies that are validated to bind the most highly conserved and important epitopes of the microorganism, e.g., immunoprotective protein target, e.g., antigen or epitope of interest. This is much faster than traditional mouse hybridoma technology, which typically takes at least 2 months before a panel of antibodies has been validated to bind the target antigen or epitope. Antibodies identified by the provided methods have significant advantages over a panel of hybridoma antibodies that come from just the most dominant B cells clones and are therefore can be nonfunctional. Additionally, hybridoma antibodies would still need to undergo the lengthy humanization process after discovery, illustrating how the provided methods for screening antibody-producing cells would save significant time (˜4 months) when responding to emerging infectious disease threats.

The provided methods for screening antibody-producing cells can fill a gap in response capability to emerging infections. Antibody therapeutics offer a safety profile that provides broad clinical applicability, able to serve the needs of pediatric and other special populations. Unlike other antibody generation technologies, provided methods for screening antibody-producing cells have a very short production cycle from B-cell to cloned antibody. This makes it suitable for responding to diseases of previously unknown etiology, where few molecular tools will be available. Of particular relevance is the fact that an infected or recovered victim of an emerging disease can provide the antibody-producing cells, e.g., B-cells, for screening using the provided methods. Further, the extraordinarily selective capacity of the provided methods, rare antibodies can be identified that other methods will miss due to being awash in antibodies lacking therapeutic potential.

In one aspect, the provided method involves an antibody discovery platform that enables the rapid generation of therapeutic candidates to address a multitude of infectious disease threats. As described above, the time required to identify antibodies of interest according to the provided methods is substantially less than using existing methods to identify antibodies of interest. Further, the provided methods allow for identification of rare antibodies that bind to conserved epitopes of interest, which is difficult using existing methods due to the presence of immunodominant, hypervariable epitopes on microorganisms. This technology may also be used to identify such antibodies targeting bacterial or fungal cells. In particular embodiments, the platform is used to generate antibodies with intrinsic bactericidal activity against multidrug-resistant Gram-negative bacteria. In particular embodiments, methods of the present invention are used to identify and obtain antibodies that specifically bind to BamA or LptD.

Advantages of the provided methods for screening antibody-producing cells include, but are not limited to:
The safety, specificity, and pharmacokinetic properties of therapeutic antibodies is well suited for the rapid development of infectious disease countermeasures
The high specificity and low off-target toxicity potential make antibodies an ideal therapeutic for high-risk patient populations such as pediatrics and the elderly.
The provided methods for screening antibody-producing cells allow generation of therapeutic antibodies to important epitopes, not possible with traditional hybridoma or phage antibody approaches
The in vivo rare cell enrichment, e.g., SCID mouse expansion, of rare functional antibodies unlocks the diversity of the entire immune repertoire
The screening of gel microdroplets that include antibody-producing cells, e.g., B cells and/or plasmablasts, is faster than fluidic systems to query large numbers of single cells
Single B cell cloning eliminates the need for a fusion partner, allowing discovery of human antibodies from any cell source; and single B cell cloning ensures proper heavy and light chain pairing, which is not possible with phage display.

A. Candidate Antibody-Producing Cells

In any of the embodiments of the methods provided herein, a plurality of candidate antibody-producing cells to be screened and identified, e.g., B cells and/or plasmablasts, can be from a variety of sources, such as donor animals and/or modified cells. In some embodiments, candidate antibody-producing cells are obtained from a donor, e.g., an animal, that has been exposed to the target microorganism or epitope-comprising fragment thereof or variant thereof and/or any combination thereof. For example, in some embodiments, the antibody-producing cells are obtained from a donor, e.g., an animal, that has been immunized with or infected with the target antigen or epitope or variant thereof, the microorganism of interest that expresses the target antigen or epitope or variant thereof, and/or any combination or mixtures thereof.

In certain embodiments, the antibody-producing cells obtained from an animal infected by or immunized with the target microorganism, e.g., pathogen, or epitope-comprising fragment thereof and expanded are peripheral blood mononuclear cells (PBMCs) or B cells or plasmablasts.

In certain embodiments, the antibody-producing cells are obtained from a human or other animal donor who was infected by the pathogen or immunized with the pathogen or an epitope-comprising fragment thereof. In some embodiments, the donor is a mammal or a bird. In some embodiments, the donor is a human, a mouse or a chicken.

In particular embodiments, human antibody producing B cells are obtained from humans or humanized animals, e.g., mice or chickens, immunized with a target pathogen or infected with a target pathogen. In particular embodiment, the pathogen is a bacteria, virus or other microbe. In some embodiments, the donor is a human donor who was infected by the microorganism.

In certain embodiment, the animal infected by or immunized with the target pathogen or epitope-comprising fragment thereof is a genetically modified non-human animal that produces partially human or fully human antibodies. Such animals are known and available in the art and include, but are not limited to e.g., transchromosomic cattle and transgenic rodents, such as the Trianni transgenic mouse, and transgenic chicken, such as the HuMab Chicken from Crystal Biosciences.

In some embodiments, the antibody-producing cells are cells that have been modified cells, e.g., genetically or physical modified. In some embodiments, the antibody-producing cells are fusion cells, e.g., hybridomas. In some embodiments, the antibody-producing cells have not been modified.

In some embodiments, enrichment of the antibody-producing cells is employed. In some embodiments, enrichment can be carried out by introducing antibody-producing cells complexed with an antigen (e.g. an epitope-comprising fragment of a target microorganism) into an immunocompromised animal, such as a SCID mouse. n certain embodiments, B cells and/or plasmablasts producing antibodies that bind the target pathogen are produced by introducing the target antigen into an immunocompromised animal, such as SCID animals, e.g., mice. In particular embodiments, the antigen is introduced into SCID animals by splenic injection or tail vein injection. Exemplary methods involving methods of B cell enrichment and expansion are described further in Section III below.

In certain embodiments, the immunocompromised animal is a rodent with severe combined immunodeficiency (SCID), e.g., a SCID mouse. Examples of immunocompromised animals that may be used according to the present invention include but are not limited to those described U.S. Patent Application Publication No. US2014/0134638, Depraeter et al. (2001) J. Immunology 166:2929-2936, PCT Patent Application Publication No. WO1999/60846, and U.S. Pat. No. 5,663,481.

In certain embodiments, the methods are used to enrich for antigen-specific plasmablasts or B cells in order to identify rare antibodies, for example, by an in vivo rare cell enrichment step. In particular embodiments, cells from the donor animal, including the antibody-producing cells, e.g., peripheral blood leukocytes or PBMCs, are introduced into the immunocompromised animal by engraftment into the animal's spleen together with antigen (e.g., target pathogen or an epitope-comprising fragment thereof). In other embodiments, they are introduced, either alone or in combination with target pathogen or epitope-comprising fragment thereof, into the immunocompromised animal parenterally, e.g., intravenously, such as by tail vein injection. In certain embodiments, the antibody-producing cells are incubated with the target pathogen or epitope-comprising fragment thereof before being introduced into the immunocompromised animal.

In some embodiments, the plurality of candidate antibody-producing cells are obtained from a library of antibody-producing cells, e.g., B cell libraries or recombinant antibody-producing cell libraries.

B. Target Microorganism or Epitope-Comprising Fragment Thereof

Provided methods can be used to rapidly and specifically identify an antibody that binds a target microorganism. In particular, the provided methods are useful for target microorganisms and/or epitope-comprising fragments thereof, or antigens thereof, in which existing methods used for antibody identification were ineffective, inefficient and/or non-specific, due to difficulties in finding rare antibody-producing cells that produce antibodies specifically targeting the microorganism, antigen or epitope of interest.

In some embodiments, the methods include encapsulating a plurality of candidate antibody-producing cells in gel microdroplets with a target microorganism.

The provided methods can be used to identify antibodies that target any microorganism of interest. For example, the target microorganism can be a pathogenic microorganism, e.g., a pathogen. The target microorganism can be a prokaryote, a eukaryote or a virus. The target microorganism can be unicellular or multicellular. In various embodiments of methods of the present invention, the pathogen is a microorganism, including but not limited to any of those described herein. In particular embodiments, the microorganism is a bacterium or a fungus. In some embodiments, the pathogen is a bacterium, a fungus, a parasite or a virus.

Examples of cells that are amenable to this invention include but are not limited to Escherichia, Klebsiella, Acenitobacter, Enterobacter, pseudomonas, Francisella, burkholderia, staphylococcus, streptococcus, Aspergillus, and Coccidia species.

In some embodiments, the target microorganism is a bacterium, e.g., a Gram negative bacterium. In some embodiments, the bacterium is a proteobacterium. For example, in some embodiments, the target microorganism is selected from among a species of Acinetobacter, Bdellovibrio, Burkholderia, Chlamydia, Enterobacter, Escherichia, Francisella, Haemophilus, Helicobacter, Klebsiella, Legionella, Moraxella, Neisseria, Pantoea, Pseudomonas, Salmonella, Shigella, Stenotrophomonas, Vibrio and Yersinia.

In some embodiments, the microorganism is selected from among Acinetobacter apis, Acinetobacter baumannii, Acinetobacter baylyi, Acinetobacter beijerinckii, Acinetobacter bereziniae, Acinetobacter bohemicus, Acinetobacter boissieri, Acinetobacter bouvetii, Acinetobacter brisouii, Acinetobacter calcoaceticus, Acinetobacter gandensis, Acinetobacter gerneri, Acinetobacter guangdongensis, Acinetobacter guillouiae, Acinetobacter gyllenbergii, Acinetobacter haemolyticus, Acinetobacter harbinensis, Acinetobacter indicus, Acinetobacter johnsonii, Acinetobacter junii, Acinetobacter kookii, Acinetobacter lwoffii, Acinetobacter nectaris, Acinetobacter nosocomialis, Acinetobacter pakistanensis, Acinetobacter parvus, Acinetobacter pitii, Acinetobacter pittii, Acinetobacter puyangensis, Acinetobacter qingfengensis, Acinetobacter radioresistans, Acinetobacter radioresistens, Acinetobacter rudis, Acinetobacter schindleri, Acinetobacter seifertii, Acinetobacter soli, Acinetobacter tandoii, Acinetobacter tjernbergiae, Acinetobacter towneri, Acinetobacter ursingii, Acinetobacter variabilis, Acinetobacter venetianus, Escherichia coli, Haemophilus influenzae, Klebsiella pneumoniae, Pseudomonas aeruginosa, Salmonella typhimurium, Shigella boydii, Shigella dysenteriae, Shigella flexneri, Shigella sonnei, Vibrio cholera and Yersinia pestis. In some embodiments, the pathogen is Acinetobacter baumannii.

In some embodiments, the target microorganism is multi-drug resistant microorganism. Any of the embodiments of the methods provided herein can be used to rapidly and effectively identify antibodies that target those target microorganisms, thereby allowing identification of new therapeutic agents that the multidrug resistant target microorganisms are susceptible to. In some embodiments, the target microorganism is multidrug-resistant Gram-negative bacteria.

In any of the methods provided herein, the antibody to be identified binds a target microorganism, in particular, an epitope-comprising fragment of the target microorganism. For example, in some embodiments, the antibody binds to an antigen expressed in the target microorganism or an epitope, in particular, on the surface of the target microorganism.

In some embodiments, the epitope-comprising fragment can be any fragment or portion of a cell that includes an epitope, which include antigenic determinants that are recognized by the immune molecules, e.g., antibodies or immune receptors. For example, in some embodiments, the epitope-comprising fragment is an antigen. In some embodiments, the epitope-comprising fragment is an epitope, or a fragment or a portion of an antigen.

In some embodiments, the epitope-comprising fragment is a protein or a polypeptide or a fragment thereof. In some embodiments, the epitope-comprising fragment is selected from among one or more of a protein, a glycoprotein, a lipid, a phospholipid, a glycolipid, a lipopolysaccharide, a nucleic acid, a polysaccharide and/or a combination thereof.

In some embodiments, the epitope-comprising fragment is present on the surface of the microorganism. In some embodiments, the epitope-comprising fragment is accessible by the identified antibody on a live microorganism, e.g., bind to an antigen or epitope on the surface of the microorganism. For example, in some embodiments, the epitope-comprising fragment is selected from among bacterial outer membrane (OM) proteins, membrane proteins, envelope proteins, cell wall proteins, surface lipids, glycolipids (e.g. lipopolysaccharide), glycoproteins, surface polysaccharides (e.g. capsule), surface appendages (e.g. flagella or pili), monomolecular surface layers (e.g. S-layer), or any epitope, portion or fragment thereof or a combination thereof. In some embodiments, the epitope-comprising fragment is associated with the outer membrane (OM), cell wall or envelope of the target microorganism. In some embodiments, the target microorganism is a Gram negative bacterium, and the epitope-comprising fragment is an OM protein. In some embodiments, the epitope-comprising fragment is associated with the extracellular side of the OM. In some embodiments, the epitope-comprising fragment is associated with the envelope of a virus, or the cell wall of a bacterium or a fungus.

In some embodiments, the epitope-comprising fragment of the microorganism, e.g., an antigen is an essential component of the target microorganism. In some embodiments, the antigen that contains the epitope-comprising fragment is an essential protein in the target microorganism. In some embodiments, binding of the antibody identified using the methods provided herein to the antigen or the epitope-comprising fragment, can result in blocking, reducing, preventing, altering and/or inhibiting the function of the epitope-comprising fragment that is an essential component of the microorganism, thereby interfering with an essential function in the target microorganism and rendering the target microorganism susceptible to therapeutic interventions using the antibody.

In some embodiments, the epitope-comprising fragment comprises an OM protein of Gram negative bacteria. OM proteins are fully integrated membrane proteins which serve essential functions for the target microorganism, including nutrient uptake, cell adhesion, cell signaling and waste export. In some target microorganisms, the OM proteins also serve as virulence factors for nutrient scavenging and evasion of host defense mechanisms. In some cases, interfering with the function of an essential OM protein in Gram negative bacteria, e.g., by binding of an antibody, can kill or severely inhibit the growth of the bacteria. In some embodiments, the epitope-comprising fragment comprises an OM protein selected from among BamA, LptD, AdeC, AdeK, BtuB, FadL, FecA, FepA, FhaC, FhuA, LamB, MepC, MexA, NalP, NmpC, NspA, NupA, Omp117, Omp121, Omp200, Omp71, OmpA, OmpC, OmpF, OmpG, OmpT, OmpW, OpcA, OprA, OprB, OprF, OprJ, OprM, OprN, OstA, PagL, PagP, PhoE, PldA, PorA, PorB, PorD, PorP, SmeC, SmeF, SrpC, SucY, TolC, TtgC and TtgF.

For selecting a target microorganism, antigen and/or epitope for antibody discovery, there are typically four key considerations upon starting a new therapeutic antibody discovery effort focused on a new infectious disease target. First, the selected target antigen or epitope within the microorganism, e.g., pathogen, of interest must be essential to fitness or viability of the pathogen. Second, it is necessary for the target to be accessible to an antibody therapeutic. Third, epitopes amenable to antibody binding must be highly conserved across the most prevalent clinical isolates of the pathogen. And finally, a strong rationale should be developed for how antibody binding to the conserved epitope would translate to inhibition of the essential target. In some embodiments, the epitope-binding fragment of a target microorganism that meet the four criteria described above is BamA, e.g., an epitope-binding fragment for antibody discovery project in A. baumannii.

In particular embodiment, the provided methods can be used to generate antibodies with intrinsic bactericidal activity against multidrug-resistant Gram-negative bacteria. In some embodiments, such antibodies require a target that is accessible to antibody engagement and for which inhibition is fatal to the cell. Recent characterization of two genes known to encode essential proteins on the surface of Gram-negative bacteria, BamA (β-barrel assembly machinery) and LptD (lipopolysaccharide transport), creates such an opportunity for the discovery of bactericidal antibodies. In some embodiments, epitope-binding fragment of a target microorganism is or comprises BamA or LptD.

Depletion of either LptD or BamA in Escherichia coli stalls assembly of the outer membrane, an essential organelle in Gram-negative bacteria, thereby causing cell death. LptD and BamA are both integral outer-membrane (OM) β-barrel proteins with critical roles in outer-membrane biogenesis. BamA is a 16-stranded β-barrel with five polypeptide transport-associated (POTRA) domains that sit in the periplasm. LptD catalyzes the terminal step in export of lipopolysaccharide to the cell surface, while BamA is required to fold all outer-membrane proteins, including LptD. LptD forms a complex with the lipoprotein LptE to form a complex in the OM. Antibody inhibition of LptD would decrease LPS levels in the outer-membrane causing dramatic sensitization to traditional antibiotics or cell death. Antibody inhibition of BamA would block folding of outer-membrane proteins thereby dramatically compromising the essential functions of the outer-membrane. LptD and BamA are ubiquitous among Gram-negative bacterial species raising the possibility that antibodies that inhibit these targets could be relevant for a broad range of Gram-negative pathogens, leading to a paradigm shift in the way Gram-negative bacterial infections are treated.

In some embodiments, the epitope-comprising fragment comprises A. baumannii BamA. In certain embodiments, the epitope-comprising fragment comprises the sequence of amino acids set forth in SEQ ID NO: 1, 2, 5, 6 or 31 or a fragment, region or domain thereof. In some embodiments, the epitope-comprising fragment comprises the sequence of amino acids comprising at least 90% sequence identity to sequence of amino acids set forth in SEQ ID NO: 1, 2, 5, 6 or 31 or a fragment, region or domain thereof, such as at least 90%, 91%, 92%, 93%, 94%, 95%, 96%, 97%, 98%, or 99% sequence identity thereto. In some embodiments, the epitope-comprising fragment used in the methods provided herein is optionally linked to an affinity tag for purification and/or a cleavage sequence for subsequent removal of the tag.

In some embodiments, the epitope-comprising fragment comprises A. baumannii LptD.

In some embodiments, the epitope-comprising fragment is a portion or a fragment of a protein or a polypeptide. In some embodiments, the epitope-comprising fragment is a polypeptide fragment or a contiguous stretch of amino acid residues, and has a length of between about 5 and about 25 amino acid residues, such as about 7 to about 22, about 9 to about 22, about 10 to about 20, about 12 to about 20, about 13 to about 19, about 14 to about 19, about 13 to about 17 amino acid residues. In some embodiments, the epitope-comprising fragment contains discontinuous (conformational) epitopes comprising polypeptide segments that are distantly separated in the protein sequence and brought into proximity by the three-dimensional folding of the protein. In some embodiments, the conformational epitope has combined length of between about has a length of between about 5 and about 25 amino acid residues, such as about 7 to about 22, about 9 to about 22, about 10 to about 20, about 12 to about 20, about 13 to about 19, about 14 to about 19, about 13 to about 17 amino acid residues.

In some embodiments, the epitope-comprising fragment comprises an epitope that is conserved across many variants of the target microorganism or across different species of microorganisms. In some embodiments, the epitope-comprising fragment comprises an epitope that is conserved across many variants of a protein expressed on the surface of a microorganism or variants thereof. Exemplary variants of A. baumannii include, but are not limited to, A. baumannii ATCC 19606, A. baumannii ATCC 17978, A. baumannii strain 1440422, A. baumannii strain MSP4-16 and A. baumannii strain 1202252.

In some embodiments, the variants are derived different clinical isolates of the same microorganism. In some embodiments, the two or more variants, e.g., variant proteins, each independently comprises an epitope-comprising fragment of the target microorganism. In some embodiments, the two or more variants, e.g., variant proteins, share at least one conserved region or domain. In some embodiments, the two or more variants each comprise at least one region or domain that differs from each other. In some embodiments, the two or more variants, e.g., protein variants, differ in length, e.g., one of the variants has a deletion of a particular region or domain of the protein. The epitope-comprising fragments used in some embodiments of the methods provided herein, can be derived from naturally occurring variants and/or can be genetically engineered or manipulated. For example, in some embodiments, the epitope-comprising fragments comprise a first variant and a second variant protein, and the first and/or second variant is a full-length and the other of the first and/or second variant is a fragment of the protein comprising deletion of an immunodominant epitope or loop of the protein. In some embodiments, the epitope-comprising fragment can be engineered to preclude antibody binding to a conserved epitope on the periplasmic portion or an intracellular portion of a membrane protein. In some embodiments, domains or regions of the epitope-comprising fragments can be swapped between different variants to result in a new variant that comprises certain domains or regions from one variant, and other domains or regions from another variant of the target microorganism. For example, in some embodiments, a variable loop containing an epitope can be swapped between different variants. For example, BamA variant 5 (set forth in SEQ ID NO:31) is a modified version of BamA variant 1, where the extracellular Loop 4, a loop that is highly variable between different isolates and variants of BamA, is replaced by the extracellular Loop 4 sequence of BamA variant 2.

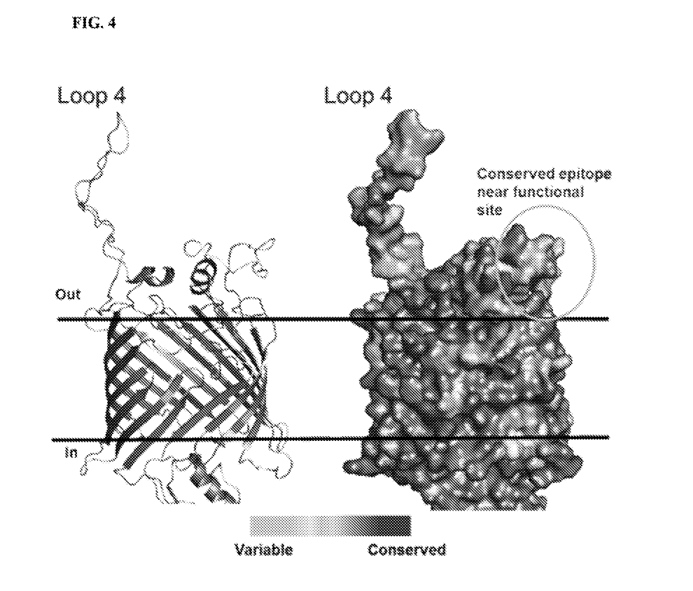

In some embodiments, the conserved epitope is an epitope that is conserved between at least two different variants of A. baumannii. In some embodiments, the conserved epitope an epitope that is conserved between at least two different variants of BamA. For example, in some embodiments, the target microorganism is A. baumannii, and a first and second variant of BamA is expressed on a first and second variant of A. baumannii. In some embodiments, the first and second variants of A. baumannii are derived from different clinical isolates. BamA contains regions or domains that exhibit significant amino acid diversity between different variants, in particular, in the extracellular loops, e.g., in loop 4 (see, e.g., FIG. 4). In some embodiments, the regions or domains that exhibit significant amino acid diversity are hypervariable and/or immunodominant regions or domains. BamA also contains conserved domains or regions, that are conserved across different variants. In some embodiments, such highly conserved domains or regions are essential or critical to the function of the protein.

In some embodiments, the conserved epitope is or comprises a contiguous sequence of amino acids. In some embodiments, the conserved epitope is or comprises a non-contiguous sequence of amino acids. For example, BamA is a transmembrane protein, and contains a periplasmic domain, transmembrane β-barrel and extracellular and periplasmic loops. For antibodies that bind to an epitope-comprising fragment on surface of the target microorganism, e.g., an OM protein, the extracellular loops are exposed on the surface of the target microorganism. Thus, such antibodies will bind to the epitopes within the exposed extracellular loops. For OM proteins that are transmembrane proteins, such as BamA, the epitope can comprise non-contiguous sequences, as the antibody can bind an epitope that comprises one or more discrete extracellular loops or portions thereof or a combination thereof.

Exemplary regions that are conserved in various A. baumannii can include amino acid residues 423-438, 440-460, 462-502, 504-533, 537-544, 547-555, 557-561, 599-604, 606-644, 646-652, 659-700, 702-707, 718-723, 735-747, 749-760, 784-794, 798-804, 806-815 and 817-841 of the A. baumannii ATCC 19606 BamA sequence set forth in SEQ ID NO:11. In some embodiments, exemplary conserve regions that are conserved in various A. baumannii include any one or more of the amino acid sequences set forth in SEQ ID NOS:12-30 or any fragments thereof.

In some embodiments, the epitope bound by the antibody identified using the methods provided herein is a conserved epitope between different variants of the microorganism, e.g., a conserved epitope on different variants of a protein expressed on the surface of the microorganism. In some embodiments, the identified antibody binds to the at least one conserved region or domain of the target microorganism. Such identified antibodies that bind to conserved epitopes can be effective against broad range of microorganism variants, e.g., pathogens of different serotypes, or a variety of pathogen species.

In some embodiments the epitope-comprising fragments thereof may be generated by expression in cell systems or grown in media that enhance protein production. In some embodiments, all or a portion of the epitope-comprising fragment can be produced using recombinant techniques. In some embodiments, the epitope-comprising fragment can be produced in recombinant bacterial or fungal protein expression systems. In some embodiments, exemplary bacterial cells that can be used for recombinant express include E. coli strains MC4100, B1LK0, RR1, E. coli LE392, E. coli B, E. coli X 1776 (ATCC No. 31537), E. coli BL21-DE3, and E. coli W3110 (F-, λ-, prototrophic, ATCC No. 273325).

In some embodiments, the epitope-comprising fragments are produced recombinantly, and are subject to purification. In some embodiments, polynucleotides encoding the epitope-comprising fragments or variants thereof, are operably linked to polynucleotides encoding an affinity tag or a purification tag, to facilitate purification. Exemplary affinity tags include polyhistidine tags (e.g., set forth in SEQ ID NO:10), Strep tag, FLAG tag, AviTag™, HA-tag, myc tag and GST tag. In some embodiments, the polynucleotides encode a fusion protein of the epitope-comprising fragment and the affinity tag. In some embodiments, purification columns are used to isolate or purify the epitope-comprising fragment from the rest of the biological material from the recombinant expression system. In some embodiments, the epitope-comprising fragment used in the methods provided herein is optionally linked to a cleavage sequence, such as a protease cleavage site. In some embodiments, protease cleavage site can be used for subsequent removal of the affinity tag. Exemplary cleavage sequence includes Tobacco Etch Virus (TEV) cleavage site. In some embodiments, the epitope-comprising fragments used in the methods provided herein are optionally linked to one or more tags and/or one or more cleavage sequences. Exemplary of such tags include AviTag-10×His-TEV (set forth in SEQ ID NO:9).

In some embodiments, the epitope-comprising fragment is a membrane protein, such as an OM protein, and the provided method comprises generating a preparation of the epitope-comprising fragments, that comprises solubilization, denaturation and/or refolding of the membrane-associated polypeptides or fragments. In some embodiments, solubilization and/or refolding requires another protein that forms a complex with the epitope-comprising fragment. For example, LptD forms a complex with the lipoprotein LptE in the OM, and a preparation of LptE is required for proper refolding of LptD. Preparations of epitope-binding fragment can be generated by standard recombinant DNA techniques and, if necessary, the epitope-binding fragments can be solubilized, such as using any of the methods known in the art or described herein. Exemplary steps for solubilization of membrane proteins include those described in WO 2015/097154.

In some embodiments, the provided method also includes refolding of the epitope-comprising fragment prior to mixing or incubating with the antibody-producing cells. In some embodiments, the refolding is carried out in the presence of one or more detergent or surfactant. In some embodiments, epitope-comprising fragments can be solubilized, denatured and/or refolded using detergents or surfactants in the preparation. In some embodiments, the solubilized and/or denatured preparations can be refolded or re-natured, e.g., in the presence of detergents or surfactants. In some embodiments, the detergent or surfactant is selected from among lauryldimethylamine oxide (LDAO), 2-methyl-2,4-pentanediol (MPD), an amphipol, amphipol A8-35, C8E4, Triton X-100, octylglucoside, DM (n-Decyl-β-D-maltopyranoside), DDM (n-Dodecyl-β-D-maltopyranoside, 3-[(3-Cholamidopropyl)dimethylammonio]-1-propanesulfonate (CHAPS) and 3-[(3-cholamidopropyl)dimethylammonio]-2-hydroxy-1-propanesulfonate (CHAPSO). In some embodiments, excess detergent in the preparation can be removed prior to immunization or contacting or incubating with antibody-producing cells.

In certain embodiments, the epitope-comprising fragment thereof is bound to a solid support, such as a bead. In certain embodiments, an antigen-containing fragment or pathogen antigen is tethered to a substrate using a suitable linking agent (e.g., a suitable ortho-nitrobenzyl-based linking agent) that possesses one or more of the following features: a tag for linking to a substrate, a spacer moiety, a linker, e.g., a cleavable linker, and a reactive group. In certain embodiments, the tag may be an affinity tag, e.g., a biotin group or the like, or a reactive moiety (e.g. a carboxy group, an amino group, a halo group, a tosylate group, a mesylate group, a reactive hydroxyl groups or metal oxide) that can react with suitable sites (e.g., alcohols, amino nucleophiles, thiol nucleophiles or silane groups on the surface of a substrate to produce a covalent bond between the substrate and the linker or the antigen-containing fragment. In certain embodiments, the spacer may contain an unreactive alkyl chain, e.g., containing 3-12 carbon atoms (e.g., 5-aminocaproic acid) and the cleavable linker may be chosen as containing appropriate chemistry (see above). The reactive group generally reacts with the effector molecule and forms a covalent bond therewith. Suitable reactive groups include halogens (that are sulhydryl reactive), N-hydroxysuccinimide (NHS)-carbonate (that are amine-reactive) and N,N-diisopropyl-2-cyanoethyl phosphoramidite (that are hydroxyl-reactive), and several other reactive groups are known in the art and may be readily employed in the instant methods.

In certain non-limiting embodiments, beads can range in size from 20 nm to 200 μm or larger. In some embodiments, the bead has an average diameter of between about 100 nm and about 100 μm, about 250 nm and about 75 μm, about 500 nm and about 50 μm, about 750 nm and about 25 μm, about 1 μm and about 10 μm, about 2 μm and about 8 μm, about 3 μm and about 7 μm or between about 3 μm and about 5 μm; or has an average diameter of about 1 μm, 2 μm, 3 μm, 4 μm, 5 μm, 6 μm, 7 μm, 8 μm, 9 μm or 10 μm.

In some embodiments, a bead may be made, e.g., of polystyrene, but other materials such as polymethylmethacrylate (PMMA), polyvinyltoluene (PVT), styrene/butadiene (S/B) copolymer, styrene/vinyltoluene (S/VT) are also used. Beads useful in the present invention can be obtained commercially from numerous sources including Molecular Probes (Invitrogen), Bangs Labs, and Polymicrospheres, Inc.

Beads can be made to display a variety of chemically functional groups on their surface. Reactive groups commonly used include carboxyl, amino, aldehyde, hydroxyl, epoxy, and chloromethyl (See, e.g., U.S. Pat. Nos. 4,217,338, 5,326,692, 5,786,219, 4,717,655, 7,445,844, 5,573,909 and 6,023,540). In certain embodiments, linkers may be attached to these reactive groups, and target antigen-containing fragments may be conjugated directly or indirectly via a linker.

C. Gel Encapsulation

The provided methods involve encapsulating the plurality of candidate antibody-producing cells in microdroplets, e.g. gel microdroplets, with a target microorganism. In some embodiments, the microdroplets comprise (i) a candidate antibody-producing cell and (ii) a target microorganism. In some embodiments, the methods further comprise encapsulating, in the microdroplets, an epitope-comprising fragment of the target microorganism or a variant thereof, e.g., an antigen or an epitope or a variant thereof of the target microorganism. In particular embodiments, microdroplets comprise: (i) one or more antibody-producing cell; and (ii) a target microorganism, e.g., pathogen, and/or an epitope-comprising fragment thereof. In some embodiments, the epitope-comprising fragment is bound to a solid support, such as a bead. The microdroplets, e.g. gel microdroplets, may comprise multiple copies of the target microorganism, e.g., pathogen, and/or epitope-comprising fragment thereof. The microdroplets, e.g. gel microdroplets, provide for a rapid and efficient method of screening antibodies that bind the target antigen, and can substantially reduce the time required to identify antibodies with desired binding specificity to a specific target, compared to any conventional methods.

In some embodiments, the plurality of candidate antibody-producing cells is selected or purified by a positive or negative selection to isolate or enrich for antibody-producing cells, e.g., B cells, plasmablasts and/or plasma cells. In some embodiments, the antibody-producing cells are plasmablasts or plasma cells. In some embodiments, the antibody-producing cells are selected or purified from an organ or a tissue sample from the donor or immunocompromised animal prior to encapsulation. In some embodiments, the organ or tissue sample is a spleen or lymph node. In some embodiments, the organ or tissue sample is peripheral blood. In some embodiments, the cells obtained from the donor or immunocompromised animal are peripheral blood mononuclear cells (PBMCs), B cells, plasma cells and/or plasmablasts.

In some embodiments, cells from the organ or tissue sample, such as the plurality of candidate antibody-producing cells are subject to one or more positive or negative selection based on expression of cell surface markers. In some embodiments, obtaining candidate antibody-producing cells includes a selection of cell types based on the expression or presence in the cell of one or more specific molecules, such as surface markers, e.g., surface proteins, intracellular markers, or nucleic acid. In some embodiments, any known method for selection based on such markers may be used to obtain candidate antibody-producing cells. In some embodiments, the selection is affinity- or immunoaffinity-based selection. For example, the isolation in some aspects includes selection of cells and cell populations based on the cells' expression or expression level of one or more markers, typically cell surface markers, for example, by incubation with an antibody or binding agent that specifically binds to such markers, followed generally by washing steps and selection of cells having bound the antibody or binding agent, from those cells having not bound to the antibody or binding agent.

Such selection steps can be based on positive selection, in which the cells having bound the reagents are retained for further use, and/or negative selection, in which the cells having not bound to the antibody or binding agent are retained.

The selection need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of selection steps are carried out, where the positively or negatively selected fraction from one step is subjected to another selection step, such as a subsequent positive or negative selection. In some examples, a single selection step can deplete cells expressing multiple markers simultaneously, such as by incubating cells with a plurality of antibodies or binding agents, each specific for a marker targeted for negative selection. Likewise, multiple cell types can simultaneously be positively selected by incubating cells with a plurality of antibodies or binding agents expressed on the various cell types.

In some embodiments, the selection is a positive selection and the cell surface marker is selected from among one or more of: CD2, CD3, CD4, CD14, CD15, CD16, CD34, CD56, CD61, CD138, CD235a (Glycophorin A) and FceRIa. In some embodiments, one or more selection steps, such as one or more separate selection step is used to obtain candidate antibody-producing cells for encapsulation and screening. In some embodiments, commercial cell selection kits, such as B cell isolation kits available from Miltenyi Biotech, EasySep™ B Cell Isolation Kit from Stemcell Technologies, CD138+ cell isolation kit from Stemcell Technologies or Dynabeads B Cells Kit, can be used to obtain candidate antibody-producing cells. Other known markers and/or methods can be used to isolate desired candidate antibody-producing cells, e.g., B cells and/or plasmablasts. In some embodiments, the plurality of candidate antibody-producing cells for encapsulation comprise CD138+ cells. In some embodiments, at least or at least about 50%, 60%, 70%, 80%, 85%, 90%, 95%, or more of the cells are plasma cells or plasmablasts and/or are CD138+ cells.

In some embodiments, the candidate antibody-producing cells are mixed with media optimized for gel encapsulation. In some embodiments, the gel encapsulation media includes cell culture media that promotes viability of antibody-producing cells, and a density gradient media that prevents sedimentation of the antibody-producing cells during encapsulation to increase efficiency of encapsulation. Exemplary density gradient media that can be used include commercially available density gradient media, such as OptiPrep™, Lymphoprep™ Polymorphprep™, Nycodenz®, Nycoprep 1.077™, Polysucrose™ 400, Ficoll®, Histodenz™, or Histopaque®.

In some embodiments, the gel microdroplet comprises a polymer matrix and/or a gel matrix. In certain embodiments, gel microdroplets comprise agarose, carrageenan, alginate, alginate-polylysine, collagen, cellulose, methylcellulose, gelatin, chitosan, extracellular matrix, dextran, starch, inulin, heparin, hyaluronan, fibrin, polyvinyl alcohol, poly(N-vinyl-2-pyrrolidone), polyethylene glycol, poly(hydroxyethyl methacrylate), acrylate polymers and sodium polyacrylate, polydimethyl siloxane, cis-polyisoprene, Puramatrix™, poly-divenylbenzene, polyurethane, or polyacrylamide. In particular embodiments, the gel micro-drops comprise a polymer matrix, which may be e.g., agarose, carrageenan, alginate, alginate-polylysine, collagen, a plant-derived gum, cellulose or a derivatives thereof (e.g., methylcellulose), gelatin, chitosan or an extracellular matrix (ECM), as described by Kleinman (U.S. Pat. No. 4,829,000), or combinations thereof. Synthetic hydrogels that may be used in the gel microdrop include but are not limited to polyvinyl alcohol, block copolymer of ethylene-vinylalcohol, sodium polystyrene sulfonate, vinyl-methyl-tribenzyl ammonium chloride and polyphosphazene.

Gel microdroplets and screening methodologies that may be used according to the present invention include any known and available in the art. Examples of gel microdroplets and screening methodologies that may be used include but are not limited to those described in U.S. Pat. Nos. 8,415,173, 8,030,095, 7,939,344, 7,413,868, and 8,445,193, U.S. Patent Application Publication Nos. US20080038755 and US20060073095, and PCT Patent Application Publication No. WO2015/038817.

In some embodiments, the microdroplets are generated by a microfluidics-based method. Exemplary microfluidics-based devices that can be used to generate the microdroplets include μEncapsulator System (Dolomite Microfluidics) and Cellena® Microencapsulator (Biomedal Lifescience).

In some embodiments, gel microdroplets comprise agarose. In some embodiments, the agarose is low gelling temperature agarose, such as an ultra-low gelling temperature agarose. In some embodiments, the low gelling temperature agarose allows for the agarose to stay liquid at lower temperatures, e.g., temperatures that permit viability of the antibody-producing cell and the target microorganism, e.g., pathogen, and thereby allow live cell and target microorganism, e.g., pathogen encapsulation. In some embodiments, the gelling temperature of the agarose used in encapsulation is such that the temperature of liquid agarose does not adversely affect viability of the antibody-producing cell and/or the target microorganism, e.g., pathogen, and gel encapsulation can be carried out in a liquid state. In some embodiments, the agarose has a gelling temperature of lower than about 35° C., such as about 30° C., about 25° C., about 20° C., about 15° C., about 10° C. or about 5° C. In some embodiments, the agarose is an ultra-low gelling temperature agarose, such as those with a gelling temperature of lower than about 20° C., about 15° C., about 10° C. or about 5° C. In some embodiments, the agarose has a gelling temperature of between about 5° C. and about 30° C., about 5° C. and about 20° C., about 5° C. and about 15° C., about 8° C. and about 17° C. or about 5° C. and about 10° C., such as about 8° C. and about 17° C.

In some embodiments, the gel encapsulation is carried out at a temperature that allows viability of the antibody-producing cells and the target microorganism, e.g., pathogen, e.g., about 37° C., about 35° C., about 30° C., about 25° C. or about 20° C.

In some embodiments of the methods provided herein, the methods include a step of incubating the microdroplets at a temperature lower than the gelling temperature of the polymer matrix and/or gel matrix, e.g., at a temperature of between about 0° C. and about 5° C., such as about 0° C., about 1° C., about 2° C., about 3° C., about 4° C., or about 5° C. In some embodiments, the incubation is for about 1 min to about 10 min, such as about 1 min, about 2 min, about 3 min, about 4 min, about 5 min, about 6 min, about 7 min, about 8 min, about 9 min, or about 10 min.

In some embodiments, the provided methods further comprise incubating the gel microdroplets at a temperature of at or about 37° C. prior to determination of binding. This step facilitates survival and antibody secretion by the antibody-producing cells, e.g., B cells or plasmablasts. In some embodiments, the gel microdroplets are incubated in growth media. The time of incubation in media can be determined based on optimal survival and antibody secretion by the antibody-producing cells. In some embodiments, the incubation is about 45 minutes to 2 hours, such as about one hour.

In some embodiments, the gel microdroplets are surrounded by a non-aqueous environment, during or after the encapsulation step. In some embodiments, the gel microdroplets comprise growth media and are surrounded by a non-aqueous environment. In some embodiments, the non-aqueous environment comprises an oil. In some embodiments, the oil is gas permeable. The presence of the gas permeable oil allows for physical separation of the microdroplets and can ensure that the secreted antibodies do not escape the non-aqueous environment, thereby resulting in a sufficiently high concentration of the antibody in the microdroplets for increased efficiency of screening. Exemplary gas-permeable oils that can be used include fluorinated oils, including but are not limited to, 3M™ Novec™ 7500 and Fluorinert FC40 (Sigma Aldrich). In some embodiments, the gel microdroplets are incubated in a non-aqueous environment after encapsulation. In some embodiments, the gel microdroplets are incubated in a non-aqueous environment at a temperature of at or about 37° C. prior to determination of binding. In some embodiments, the non-aqueous environment comprises a gas-permeable oil, such as 3M™ Novec™ 7500 or Fluorinert FC40.

In some embodiments, the microdroplet comprises one or more target microorganism, e.g., pathogen or one or more epitope-comprising fragment of the target microorganism, e.g., pathogen or a variant thereof. In some embodiments, the microdroplet comprises one or more target microorganism, e.g., pathogen and one or more epitope-comprising fragment of the target microorganism, e.g., pathogen or a variant thereof. In some embodiments, the target microorganism, e.g., pathogen in the microdroplet expresses the epitope or variant thereof on the surface of the target microorganism, e.g., pathogen. In some embodiments, the epitope-comprising variant thereof is bound to a solid support, such as a bead. For example, in some embodiments, the microdroplets comprise one or more beads that are coated with the epitope-comprising fragment.

In some embodiments, the microdroplet comprises antibody-producing cells. In some embodiments, the microdroplets, on average, comprise one or fewer antibody-producing cells. In some embodiments, the average ratio of candidate antibody-producing cell per gel microdroplet is less than or less than about 1. In some embodiments, the average ratio of candidate antibody-producing cell per gel microdroplet is between about 0.05 and about 1.0, about 0.05 and about 0.5, about 0.05 and about 0.25, about 0.05 and about 0.1, about 0.1 and about 1.0, about 0.1 and about 0.5, about 0.1 and about 0.25, about 0.25 and about 1.0, about 0.25 and about 0.5 or 0.5 and about 1.0, each inclusive. In some embodiments, t the average ratio of candidate antibody-producing cells per microdroplet is or is about 0.1.

In some embodiments the microdroplets may contain a single antibody-producing cell and multiple target microorganism, e.g., pathogens. In some embodiments, the microdroplets may contain a single antibody-producing cell and multiple epitope-comprising fragment of the target microorganism, such as epitope-comprising fragments that are bound to solid support, e.g. beads.

The number of antibody-producing cells and the target microorganism, e.g., pathogen and/or epitope-comprising fragments may be controlled by Poisson statistics, e.g., as described in Powell (Biotechnology 1990 8: 333-7); Weaver et al (Biotechnology 1991 9: 873-877). During the encapsulation process, the components particles (e.g., antibody-producing cells, target microorganism, e.g., pathogens, epitope-comprising fragments, e.g., those bound to solid support) are randomly distributed into the nascent microdropletlets. Since virtually all of the particles become embedded in microdroplets, if the number of particles exceeds the number of microdroplets, each microdroplet may contain, on average, >1 particle. Likewise, if the number of microdroplets exceeds that of the particles, then each microdroplet may contain, on average, <1 particle.

In general, for some of the methods described herein, it may be desirable to have one or fewer antibody-producing cell per microdroplet since this ensures the encapsulation of a single type of antibody-producing cell that may act upon the target microorganism, e.g., pathogen and/or the epitope-comprising fragments, and thus generate a result that is more clearly interpretable than if multiple types of antibody-producing cells were present in the microdroplet. In some instances, microdroplets may contain antibody-producing cells that will be allowed to grow over time, resulting in multiple antibody-producing cells per microdroplet. In this case, the cells in one microdroplet would be clonal in origin, and hence only produce one type of antibody.

In some embodiments, the ratio of candidate cells to target microorganism, e.g., pathogens to epitope-comprising fragments, and the average number of each in a microdroplet, can be optimized based on the desired method of screening, detection and identification and the parameters of gel encapsulation. Exemplary variables for consideration for such optimization include, but are not limited to, e.g., size of the target microorganism, size of the microdroplet, number of other particles in the microdroplet, strength of the detection signal, antibody output of the antibody-producing cells and affinity of the antibodies. With respect to the target microorganism, e.g., pathogens and/or epitope-comprising fragments, it may be desirable to have multiple members of each type contained within each microdroplet. In some embodiments, the average ratio of the candidate cell to microorganism to bead is about 0.1:100:10. In some embodiments, the average ratio of the candidate cell to microorganism to bead is about 0.1:200:5, or about 0.1:50:20.

The number of target microorganism, e.g., pathogen per microdroplet can be optimized to ensure visibility of signal during the screening and identification of antibody-producing cells, and in relation to the size of the microdroplet. In the case of target microorganism that is a bacterium, the average number of target microorganism, per microdroplet can be between about 5 and about 500, such as about 10 and about 250, about 50 and about 200, about 50 and about 150, about 50 and about 100, or about 80 and about 120, such as about 10, 20, 30, 40, 50, 60, 70, 80, 90, 100, 110, 120, 130, 140, 150, 160, 170, 180, 190 or 200. The number of target microorganism per microdroplet may be lower on average for microorganisms that are larger in cell size, e.g., a fungus or a parasite.

The number of epitope-comprising fragments, e.g., those bound to solid support per microdroplet can be optimized for fluorescent signal sensitivity and specificity. Exemplary variables for consideration include, but are not limited to e.g., size of the bead, number of other particles in the microdroplet, size of the microdroplet, strength of the detection signal, antibody output of the antibody-producing cells and affinity of the antibodies. For example, for epitope-comprising fragments that are coated on beads, the average ratio of the bead per gel microdroplet can be between about 2 and about 25, about 3 and about 8, about 3 and about 7, about 3 and about 5, about 5 and about 20, about 5 and about 15, about 7 and about 15, about 8 and about 12, about 9 and about 11, or about 3, 4, 5, 6, 7, 8, 9, 10, 11, 12, 13, 14, 15, 16, 17, 18, 19 or 20.

D. Detecting or Identifying Antibody-Producing Cells

In some embodiments of the methods provided herein, the methods involve determining whether the antibody-producing cell(s) within the gel microdroplet produce an antibody that binds the target microorganism. Such steps can allow identification of an antibody that specifically binds to the target microorganism. In particular, in some embodiments, the methods provided herein can identify antibodies that are difficult to identify using conventional method. In some embodiments, the provided methods can be used to identify antibodies against target epitopes that are difficult to identify using conventional methods.

In some embodiments, such steps for determining binding includes determining whether the antibody identified as binding the target microorganism also binds the epitope-comprising fragment thereof within the same gel microdroplet. In some embodiments, the steps for determination of binding include methods and/or assays that detect presence of binding of molecules, e.g., binding of the antibody to an epitope-comprising fragment of a target microorganism. In some embodiments, the provided methods include methods and/or assays that detect binding, modification of a phenotypic characteristic of the target microorganism and/or death or viability of the target microorganism. In some embodiments, determination of binding and/or the downstream effects thereof, such as modification of a phenotypic characteristic of the target microorganism and/or death or viability, are carried out within the gel microdroplet, and/or using a reporter molecule.

In some embodiments, the provided method comprises a step of introducing into the gel microdroplets a reagent that binds to the antibodies prior to determining whether an antibody-producing cell within a gel microdroplet produces an antibody that binds the target microorganism, said reagent comprising a detectable moiety. For example, the reagent comprises a secondary antibody specific for antibodies produced by the encapsulated antibody-producing cells.

In particular embodiments, gel microdroplets comprise a detectable moiety that facilitates the detection of antibodies that bind the target pathogen or epitope-comprising fragment thereof. In certain embodiments, the detectable moiety specifically binds to antibodies produced by the encapsulated antibody-producing cell. In certain embodiments, the detectable moiety is a labeled secondary antibody specific for antibodies produced by the encapsulated antibody-producing cells. Antibody-producing cells, e.g., B cells and/or plasmablasts, from any species can be used in this technology by simply varying the fluorescent secondary antibody such that it is specific for the primary antibody produced by the B cell.

In particular embodiments, determining whether the antibody-producing cell(s) within the gel microdroplet produce an antibody that binds the target microorganism, e.g., pathogen and/or epitope-comprising fragment thereof present in the same gel microdroplet comprises detecting the presence of a complex comprising: (i) the target microorganism, e.g., pathogen, or epitope-comprising fragment thereof; (ii) the antibody produced by the antibody-producing cell; and (iii) the detectable moiety, wherein the presence of the complex indicates that the antibody specifically binds the target pathogen or epitope-comprising fragment thereof. In some embodiments, the determining binding can be carried out by addition of a labeled secondary antibody, e.g., antibody that can bind to primary antibodies produced by the candidate antibody-producing cells. For example, in some embodiments, the secondary antibody can detect presence of and/or binding of a primary antibody against a specific epitope, e.g., a conserved epitope, and the presence of the secondary antibody indicates the presence of the primary antibody and/or the binding of the primary antibody to the targeted microorganism and/or epitope-comprising fragment thereof. In some embodiments, the secondary antibody comprises a detectable label.

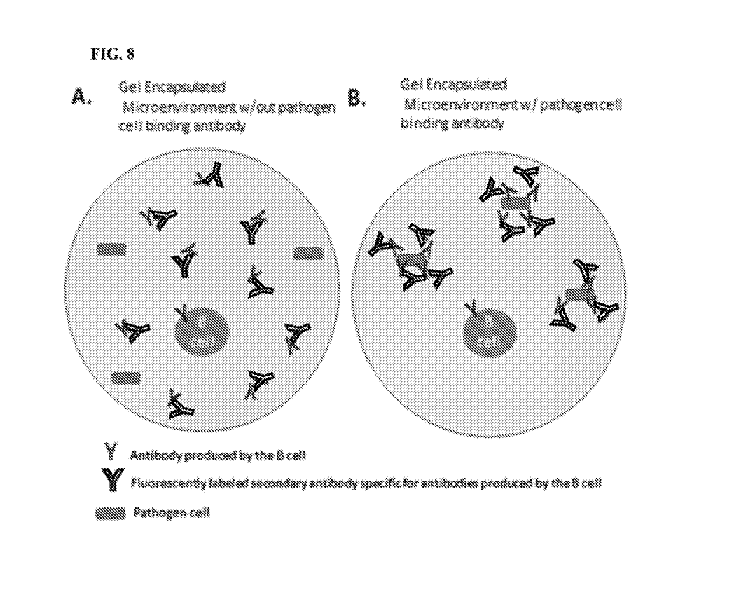

For example, in one exemplary embodiment illustrated in FIG. 8, single antibody-producing cells, e.g., B cells and/or plasmablasts, from any source can be encapsulated individually within a gel microenvironment. The antibody-producing cells, e.g., B cells and/or plasmablasts, will secrete a primary antibody which will accumulate within the gel encapsulated microenvironment. Any target microorganism, e.g., bacterial or fungal cell of interest, can be encapsulated within the same gel microenvironment. Additionally, a secondary fluorescent antibody specific for the primary antibody isotype produced by the antibody-producing cells, e.g., B cells and/or plasmablasts, can also be encapsulated within the gel microenvironment. The primary antibody will be engaged by the secondary antibody to form a fluorescent antibody complex. If the primary antibody has no binding specificity for the bacterial or fungal cell, the fluorescent antibody complexes will remain diffuse, which can be detected by the use of a fluorescent microscope to analyze the individual gel microenvironments, e.g., as lacking a spot and/or a discrete punctuate signal from the detectable label. As shown in FIG. 8, if the primary antibody binds to the surface of the target microorganism, e.g., bacterial or fungal cell, the fluorescent antibody complex will form discrete punctuate fluorescent spots within the gel microenvironment, which can be detected by using a fluorescent microscope, and this B cell will be selected for downstream processing and antibody discovery.

In certain embodiments, determining whether the antibody-producing cell(s) within the gel microdroplet produce an antibody that binds the target microorganism, e.g., pathogen and/or epitope-comprising fragment thereof present in the same gel microdroplet comprises determining whether the presence of the antibody modifies a phenotypic characteristic of the target pathogen in the same gel microdroplet, wherein the presence of the modified phenotypic characteristic indicates that the antibody specifically binds the target pathogen or epitope-comprising fragment thereof. In particular embodiments, the modified phenotypic characteristic is cell growth or cell death.

In some embodiments, the methods provided herein, the modified phenotypic characteristic is selected from among cell growth, cell death, changes in in behavior, binding, transcription, translation, expression, protein transport, cellular or membrane architecture, adhesion, motility, cellular stress, cell division and/or cell viability.

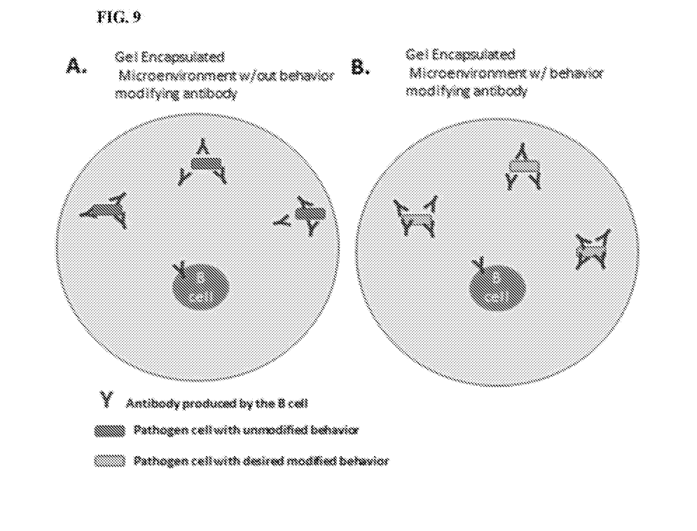

For example, in an embodiment illustrated in FIG. 9, a single antibody-producing cell, e.g., B cells and/or plasmablasts, is encapsulated within a microenvironment with the target microorganisms, e.g., bacterial or fungal cells, that are engineered to report on the cellular status of interest. For example, to obtain an antibody that causes cellular stress, the bacterial or fungal cells are engineered to change fluorescent properties upon stress induction. These engineered reporter strains could produce a fluorescent compound upon stress induction or alternatively become labile to a given chemical that under stress causes a florigenic change that can be detected by fluorescent microscopy. If the antibody does not engage the target microorganisms, e.g., bacterial or fungal cell, no observable phenotypic change will occur within the bacterial cell and those B cells will not be of interest. The antibody could make specific contact with the target microorganisms, e.g., bacterial or fungal cells, but not elicit the desired bacterial or fungal phenotype and again will not be of interest. However, as shown in FIG. 9, if the antibody binds specifically to the target microorganisms, e.g., bacterial or fungal cells, and modulates a desired behavior, that antibody-producing cell, e.g., B cells and/or plasmablasts, will be selected for downstream processing and antibody discovery. As described above, a fluorescent secondary antibody specific for the primary isotype produced by the antibody-producing cell, e.g., B cells and/or plasmablasts, could be added to simultaneously detect binding to the target microorganisms, e.g., bacterial or fungal cells, and behavior modification in the target microorganism, e.g., a bacteria or fungus.

In some embodiments, the methods provided herein involve determining whether the antibody-producing cell(s) within the gel microdroplet produce an antibody that binds the target microorganism and/or epitope-comprising fragment thereof present in the same gel microdroplet, which includes detecting a signal produced by a reporter molecule, wherein the signal is produced in the presence of the modified phenotypic characteristic.

In some embodiments, the microorganism used in the methods provided herein comprises a polynucleotide encoding a reporter molecule. For example, in some embodiments, the microorganism that is encapsulated in the gel microdroplets with antibody-producing cells are genetically engineered to contain polynucleotides that produces a reporter molecule, e.g., detectable reporter molecule, in response to a particular physiological stimulus or in a particular cellular state. By genetically engineering the microorganism, e.g., bacterial or fungal cell, to report on the cellular state of interest, rapid identification of behavior modifying antibodies may also be easily detected.

In some embodiments, the polynucleotide comprises a regulatory region operably linked to a sequence encoding the reporter molecule, wherein the regulatory region is responsive to the modified phenotypic characteristic. In some embodiments, the regulatory region comprises a promoter. For example, in some embodiments, the regulatory region is responsive to specific modified phenotypic characteristics. In some embodiments, the regulatory region is responsive to, e.g., directs modification of expression of the reporter molecule operably linked thereto, in the presence of the modified phenotypic characteristic, e.g., of the target microorganism.

In some embodiments, the modified phenotypic characteristic comprises cellular stress and the signal is produced in the presence of the cellular stress. In some embodiments, the cellular stress comprises stress to the outer membrane (OM) of the bacterium.

One of skill in the art can readily determine whether expression of a gene is modulated in the presence of a modified phenotypic change. For example, one can compare transcription levels of genes of a microorganism that has been exposed to the modified phenotypic characteristic. In some embodiments, expression levels can be assessed or determined using any method known to a skilled artisan, such as by using quantitative PCR, microarrays, RNA-Seq, northern blotting, or SAGE. In some embodiments, genes whose sequences, or portions or fragments of sequences, have been identified as having been modulated (e.g. increased or decreased) can be identified using a reference sequence of the microorganism genome. Exemplary genome sequences of microorganisms are known and readily available online on the world wide web at tigr.org, kegg.jp, or ncbi.nlm.nih.gov/genbank.

In some embodiments, the regulatory region or portion thereof comprises a sequence upstream or 5′ of the open reading frame (ORF) of a gene whose expression is modulated (e.g. increased) in response to the modified phenotypic change. In some embodiments, the sequence of the regulatory region or portion thereof is sufficient to provide for regulated expression of the coding region of the reporter molecule operatively linked thereto, such as upon induction or in the presence of the modified phenotypic change. It is within the level of a skilled artisan to carry out recombinant DNA techniques, including deletional analysis, to determine or identify regulatory region sequences, or portions thereof, sufficient to induce expression of the reporter molecule under different conditions. In some embodiments, the regulatory region is or comprises a native promoter.

One of skill in the art can identify a regulatory region through standard techniques. For example, one could identify a regulatory region by fusing a putative regulatory region or sequence to a sequence encoding a reporter molecule, introducing the construct using standard techniques into the microorganism, inducing the putative regulatory region or upstream sequence by causing the modified phenotypic change, and determining if the reporter molecule is induced. Putative regulatory regions can often be shortened or lengthened without influencing activity or inducibility. One of skill in the can systematically test the effect of removing nucleotides from putative regulatory region sequence to determine what putative regulatory elements are required or sufficient for the modified phenotypic behavior.

In some embodiments, the detectable label is selected from among a chromophore moiety, a fluorescent moiety, a phosphorescent moiety, a colorimetric moiety, a luminescent moiety, a chemiluminescent moiety, a light absorbing moiety, a radioactive moiety, and a transition metal isotope mass tag moiety. For example, in some embodiments, any fluorophores, e.g., a fluorescent moiety, that are detectable by fluorescent microscopy can be used as a readout for bacterial or fungal cell behavior modification or cell death.

In some embodiments, the detection is carried out using an apparatus selected from among a light microscope, a fluorescent microscope, a spectrophotometer, a fluorescence-activated cell sorter, a fluorescent sample reader, a 3D tomographer or a camera.

In some embodiments, the signal produced by the reporter molecule is detected with a detectable moiety. In some embodiments, the signal produced by the reporter molecule comprises a fluorescent signal, a luminescent signal, a colorimetric signal, a chemiluminescent signal or a radioactive signal. In some embodiments, the reporter molecule is a fluorescent protein, a luminescent protein, a chromoprotein or an enzyme.

In some embodiments of the methods provided herein, determining whether the antibody-producing cell(s) within the gel microdroplet produce an antibody that binds the target microorganism and/or epitope-comprising fragment thereof present in the same gel microdroplet comprises determining whether the presence of the antibody kills the target microorganism in the same gel microdroplet, wherein killing of the target microorganism indicates that the antibody specifically binds the target microorganism or epitope-comprising fragment thereof. In some embodiments, the gel microdroplets comprise a detectable moiety indicative of cell death.

In certain embodiments, determining whether the antibody-producing cell(s) within the gel microdroplet produce an antibody that binds the target microorganism, e.g., pathogen and/or epitope-comprising fragment thereof present in the same gel microdroplet comprises determining whether the presence of the antibody kills the target pathogen in the same gel microdroplet, wherein killing of the target pathogen indicates that the antibody specifically binds the target pathogen or epitope-comprising fragment thereof. In some embodiments, the gel microdroplets comprise a detectable moiety indicative of cell death. In certain embodiments, the detectable moiety is capable of distinguishing between living and dead cells, e.g., a vital dye. In particular embodiments, the gel microdroplets comprise a detectable moiety indicative of cell death. By using fluorescent dyes that distinguish live from dead bacterial or fungal cells, rapid identification of bactericidal or fungicidal antibodies could be indentified.

In some embodiments, the detectable moiety emits a signal depending on the viability of the cell, e.g., is a dye or a kit including a dye indicative of cell viability and/or death. Exemplary detectable moieties indicative of cell death include, but are not limited to: 4′,6-diamidino-2-phenylindole, 5-carboxyfluorescein diacetate, 5-cyano-2,3-ditolyl tetrazolium chloride (CTC), 7-AAD, acetoxymethyl ester (CFDA AM), an indicator of membrane integrity, Aqua-fluorescent reactive dye, BacLight Bacterial Membrane Potential Kit, BacLight mounting oil, BacLight RedoxSensor CTC Vitality Kit, BacLight RedoxSensor Green Vitality Kit, Bacteria Counting Kit (Assays for Cell Enumeration, C12-resazurin, Calcein AM, calcein AM ethidium homodimer-1, Calcein Blue AM, Calcein Violet AM, Calcofluor White M2R, Carbonyl cyanide 3-chlorophenylhydrazone (CCCP), CCCP in DMSO, Cell Proliferation and Cell Cycle—Section 15.4), CellTrace calcein violet AM, DAPI, DEAD Red nucleic acid stain, Detailed protocols (Product Information Sheet), dihydrochloride (DAPI), Dimethylsulfoxide (DMSO), DiOC18, DiOC2 in DMSO, DMSO, Dodecylresazurin (C12-resazurin), Ethidium homodimer-1, F34953, Fixable Viability Dye eFluor® 450, Fixable Viability Dye eFluor® 455UV, Fixable Viability Dye eFluor® 506, Fixable Viability Dye eFluor® 520, Fixable Viability Dye eFluor® 660, Fixable Viability Dye eFluor® 780, Fluorescent reactive dye, FUN 1 cell stain, FungaLight CFDA AM/Propidium Iodide Yeast Vitality Kit for flow cytometry, Hexidium iodide, LIVE BacLight Bacterial Gram Stain Kit, LIVE/DEAD Cell Vitality Assay Kit, LIVE/DEAD Cell-Mediated Cytotoxicity Kit, LIVE/DEAD Fixable Dead Cell Stain Kits, LIVE/DEAD Reduced Biohazard Cell Viability Kit #1, LIVE/DEAD Sperm Viability Kit, LIVE/DEAD Viability/Cytotoxicity Kit, LIVE/DEAD Yeast Viability Kit, LIVE/DEAD BacLight Bacterial Viability and Counting Kit, LIVE/DEAD BacLight Bacterial Viability Kit, LIVE/DEAD FungaLight Yeast Viability Kit for flow cytometry, LIVE/DEAD® Fixable Aqua stain, LIVE/DEAD® Fixable Blue stain, LIVE/DEAD® Fixable Violet stain, LIVE/DEAD® Fixable Yellow stain, Reaction buffer, Reaction mixture, RedoxSensor Green reagent, Resazurin, Resorufin, Sodium azide, Sodium bicarbonate, Suspended microsphere standard, SYBR 14 nucleic acid stain, SYBR™ 14 dye, SYTO 10 nucleic acid stain, SYTO 24 green-fluorescent nucleic acid stain, SYTO 9 nucleic acid stain, SYTO BC bacteria stain, SYTOX Green nucleic acid stain, SYTOX™ Green dye, Texas Red-X conjugate of wheat germ agglutinin (WGA), ViaGram Red+Bacterial Gram Stain and Viability Kit, Vybrant Cell Metabolic Assay Kit and Vybrant Cytotoxicity Assay Kit.

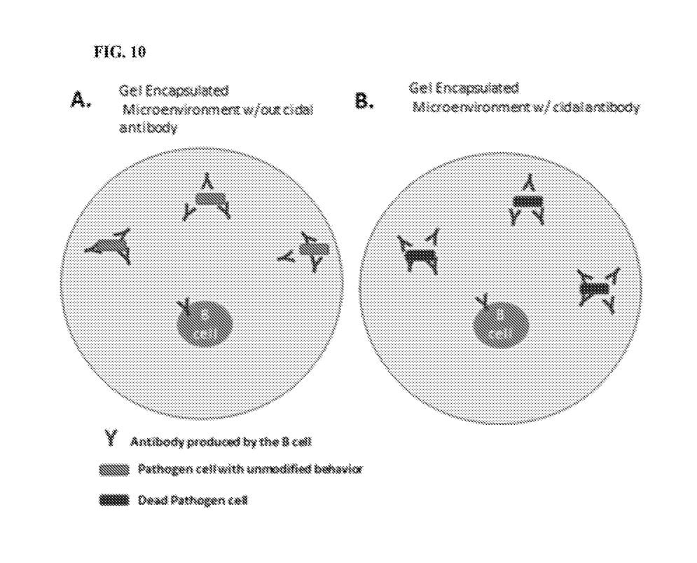

In an exemplary embodiment illustrated in FIG. 10, a single antibody-producing cell, e.g., B cell and/or plasmablast, is encapsulated within a microenvironment with a target microorganism, e.g., bacterial or fungal cells. However, also within this microenvironment is a dye that specifically enters and concentrates within cells that are dead versus cells that are alive. Therefore, dead target microorganism, e.g., bacterial or fungal cells, can be detected using microscopy. Antibodies can then be rapidly screened for their ability to cause a cidal phenotype on the target microorganism, e.g., bacterial or fungal cell of interest. If the antibody does not engage the target microorganism, e.g., bacterial or fungal cell, no observable phenotypic change will likely occur within the bacterial cell and those B cells will not be of interest. In particular embodiments, the antibody could make specific contact with the target microorganism, e.g., bacterial or fungal cells, but not elicit the cidal response. However, if the antibody binds specifically to the target microorganism, e.g., bacterial or fungal cell and causes cell death, that antibody-producing cell, e.g., B cell and/or plasmablast may be selected for downstream processing and antibody discovery. A fluorescent secondary antibody specific for the primary isotype produced by the antibody-producing cell, e.g., B cell and/or plasmablast, could be added in order to simultaneously detect bacterial or fungal cell binding and bacterial or fungal cell death. The provided methods are not limited to a dye molecule to detect cell death, as any reporter system could be used to specifically identify antibody-producing cell, e.g., B cells and/or plasmablasts, that produce cidal antibodies, e.g., antibodies that can cause a death of the target microorganism. In some embodiments, the antibodies are bactericidal antibodies.

The present disclosure also allows a functional output to be determined prior to expending time and resources necessary to clone, transiently express, purify, and test the antibody for function. Therefore, only the heavy and light chain genes from those antibody-producing cells, e.g., B cells and/or plasmablasts, previously determined to be making a functional antibody of interest will be progressed to the cloning phase. In some cases, this can save considerable time and money in the quest for rare functional antibodies, and can facilitate efficient screening of antibody-producing cells to rapidly and effectively identify antibodies of interest.

E. Isolation and Identification of Antibodies

In some embodiments, the provided methods include isolating the microdroplet comprising the cell producing the identified antibody or isolating polynucleotides encoding the antibody identified as specifically binding the target microorganism or epitope-comprising fragment thereof. In some embodiments, the provided methods also include determining the sequence of the nucleic acids encoding the identified antibody.

In some embodiments, the gel microdroplet that contains the cell producing the identified antibody, e.g., antibody of interest that binds to a target microorganism or epitope-comprising fragment thereof, is separated away from the plurality of microdroplets. In some embodiments, the isolation is carried out using a micromanipulator or an automated sorter. For example, in some embodiments, the gel microdroplets are visually screened under a microscope, e.g., under a fluorescence microscope, and the microdroplet that contains the cell producing the identified antibody, e.g., antibodies that exhibit particular desired properties as described herein, can be physically separated from other microdroplets as they are identified during the screening process. In some embodiments, the microdroplets are separated using a micromanipulator. In some embodiments, automated sorters can be used to sort particular droplets based on a criterion, e.g., level of detectable signal in the microdroplet.

Other technologies such as FACS, allow single B cell manipulations. However, FACS requires the antibody to be expressed and remain attached to the B cell surface in order to query antigen binding. Because of the high physical sheer forces during FACS, it is impossible to use FACS to isolate B cells that make antibodies that bind to the surface of bacterial or fungal cells. Therefore, the present invention described here allows the user to agnostically identify antibodies that bind to the surface of the microorganism, e.g., bacterial or fungal and elicit a cellular response. Such depth of knowledge about the antibody being produced by a B cell is not feasible with FACS alone.

In some embodiments, the provided methods also include determining the sequence of the nucleic acids encoding the identified antibody. In some embodiments, determining the sequence of the nucleic acids is carried out using nucleic acid amplification and/or sequencing. Any methods known in the art to determine the sequence of nucleic acids can be used in the art. In particular, techniques that allow determination of nucleic acid sequences from a small amount of starting material, such as single cell PCR, can be used to determine the sequence of the antibody produced by the cell contained in the gel microdroplet. In particular embodiments, the antibody from the B cell within microenvironments of interest can be identified by reverse transcription (RT)-PCR, proteomics, or any other downstream methods used to obtain the molecular signature of the antibody. In some embodiments, determining the sequence of the nucleic acids is carried out using single cell PCR and nucleic acid sequencing. In particular embodiments, provided methods further comprise isolating polynucleotides encoding the antibody identified as specifically binding the target microorganism, e.g., pathogen, or epitope-comprising fragment thereof (or fragments thereof), subcloning the polynucleotides into an expression vector, and producing recombinant antibodies that specifically bind the target pathogen.

Any gel microenvironment, e.g., gel microdroplet, identified as harboring a cell, e.g., B cell and/or plasmablast, that produces an antibody of interest as described above, can be retrieved and the antibody encoding heavy and light chain genes of the antibody-producing cell, e.g., B cell and/or plasmablast, can be PCR amplified, cloned, sequenced, and expressed according to established protocols. For example, in some embodiments, the methods provided herein also include introducing a polynucleotide comprising the sequence of the nucleic acids encoding the identified antibody or fragment thereof into a cell. In some embodiments, the polynucleotide can be introduced into a mammalian cell. In some embodiments, the polynucleotide can be introduced into a cell for recombinant expression. In some embodiments, the polynucleotide includes sequences that encode

In certain embodiments, the present invention provides a rapid method of producing the recombinant antibody by transfecting mammalian cells with the linear PCR DNA product that encodes the antibody. This eliminates the time consuming step of plasmid cloning prior to antibody production. It typically takes 10 days for plasmid cloning and verification before mammalian cell transfection can begin to make the antibody protein of interest. By being able to transfect mammalian cells with the linear PCR product, methods of the present invention may be used to begin producing antibodies within the same day that the PCR product is generated. The ability to PCR amplify the antibody genes from a single antibody-producing cell, e.g., B cell, and also transfect those genes as linear DNA product reduces the amount of time between B cell generation and therapeutic antibody generation by at least 17 days.

These antibodies can then be used to test in vitro and in vivo activity and efficacy on the specific microorganism, e.g., bacterial or fungal cell, used for detection. In some embodiments, the in vitro and in vivo activity and efficacy of such antibodies can also be tested on other variants of the same microorganism or other species of microorganisms.

The identified antibody can be further tested and evaluated for its activity. In some embodiments, the identified antibody is tested for binding to a broad range of targets, e.g., binding to many variants of the microorganism or epitope-comprising fragment thereof, and/or binding to a conserved epitope, e.g., an epitope that is conserved between many variants of the microorganism or epitope-comprising fragment thereof. In some embodiments, the antibody is tested for its functional activity, e.g., killing activity against the target microorganism, and/or ability to modify the phenotypic characteristics of the target microorganism. In some embodiments, the antibody is tested for antimicrobial activity, bactericidal activity and/or fungicidal activity. In some embodiments, the antibody is tested for its ability to induce complement fixation. In some embodiments, the antibody is tested for its functional activity against a broad range of targets, e.g., many variants of the microorganism or epitope-comprising fragment thereof and/or broad range of microorganism variants, e.g., pathogens of different serotypes, or a variety of pathogen species. In some embodiments, the antibody is tested for broadly neutralizing activity.

### III. In Vivo Rare Cell Enrichment

Some embodiments of the methods provided herein can include an in vivo rare cell enrichment step, to allow preferential stimulation and expansion of rare antibody-producing cells in vivo. In some embodiments, the in vivo rare cell enrichment step can be used to enrich for antigen-specific plasmablasts or B cells in order to identify rare antibodies. In particular, rare cells that produce antibodies that bind to a conserved epitope on the surface of a target microorganism, e.g., a non-immunodominant conserved epitope, can be preferentially stimulated using this method, greatly increasing the probability of identifying such rare cells using the methods.

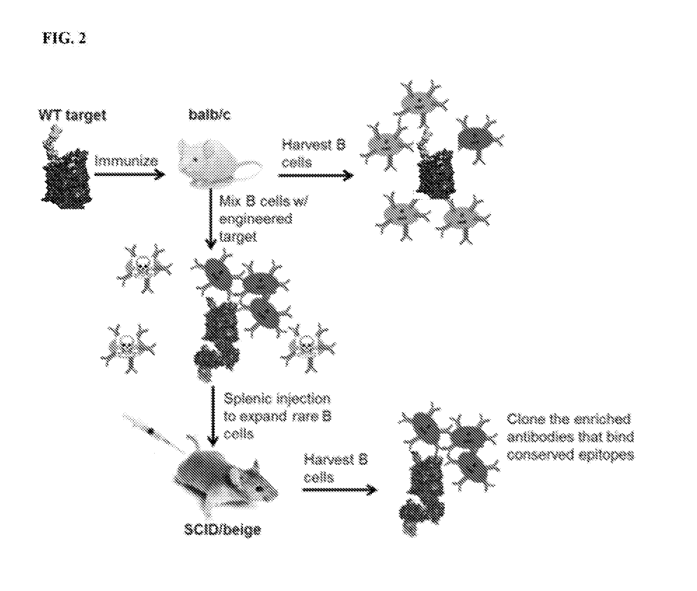

In certain embodiments, the in vivo rare cell enrichment step, e.g. rare B cell enrichment phase, involves generating a pool of candidate antibody-producing cells, e.g., B cells and/or plasmablasts, that are highly enriched for their ability to make antibodies against the immunoprotective protein of interest, e.g., an epitope-comprising fragment of a target microorganism. Because this technology does not rely on traditional hybridoma or phage display technologies, the pool of candidate antibody-producing cells, e.g., B cells, used for enrichment can come from any source. For example, the B cells utilized during this phase could be retrieved from a human subject who fell victim to infection, a human subject who has recently recovered from infection, or a humanized animal, e.g., an animal genetically engineered to produce humanized antibodies, that has been immunized with the target antigen, e.g. epitope-comprising fragment of a target microorganism. Regardless of the source, the candidate antibody-producing cells, e.g., B cells, can be expanded and enriched in an antigen specific manner within the spleen of an irradiated immunocompromised animal, e.g., SCID mouse (e.g., see FIG. 2).

In certain embodiments, methods of the present invention preferentially allows expansion of those candidate antibody-producing cells, e.g., B cells, that make antibodies to the most highly conserved epitopes of the immunoprotective target protein, antigen or epitope of the target microorganism, e.g., pathogen. Therefore, the technology enriches for antibodies that have the highest potential to bind important, critical or essential epitopes on the pathogen surface and have the highest likelihood of broad pathogen neutralization. This aspect sets the platform apart from more traditional technologies that query panels of antibodies, of which the majority do not have specificity for the target antigen of interest or bind to highly variable non-functional epitopes of the target antigen. While this traditional approach can be effective, it is incredibly labor intensive and slow, which limits its usefulness when responding to emerging infectious disease threats.

In some embodiments of the methods, the plurality of candidate antibody-producing cells is obtained by a method comprising: (i) expanding antibody-producing cells obtained from a donor that has been exposed to the target microorganism or an epitope-comprising fragment of the target microorganism or a variant thereof by introducing a cell composition comprising the antibody-producing cells into an immunocompromised animal; and (ii) recovering the expanded antibody-producing cells, thereby obtaining the plurality of candidate antibody-producing cells.

In some embodiments, the cell composition comprising the antibody-producing cells comprises cells obtained from the spleen and/or lymph node of the donor animal, such as an animal infected with or immunized with the target microorganism. In some embodiments, the cells obtained from the spleen and/or lymph node include peripheral blood mononuclear cells (PBMCs) comprising antibody-producing cells, e.g., B cells or plasmablasts, T cells, and NK cells, dendritic cells, and other immune cells. In some embodiments, the cell composition comprises T cells. Such cell compositions comprising the antibody-producing cells can be introduced to an immunocomprised animal, such as a severe combined immunodeficiency (SCID) mouse. In some embodiments, the cell composition is introduced parenterally, e.g., intravenously, such as by tail vein injection, or by transplant into the immunocompromised animal's spleen.

In some embodiments, the in vivo rare cell enrichment also includes a step of stimulating the cell composition from the donor animal with the target microorganism or a specific epitope-comprising fragment thereof, antigen or epitope or any variant thereof, prior to introducing the cell composition into the immunocompromised animal. In some embodiments, the candidate antibody-producing cells are contacted with or incubated with the target microorganism, target antigen or an epitope thereof and/or a variant of the target antigen or an epitope thereof. In some embodiments, the candidate antibody-producing cells are contacted with a mixture of one or more target microorganisms and/or variant antigens and/or epitopes, such as a mixture of different antigen variants. In some embodiments, the candidate antibody-producing cells are contacted with a target microorganism variant that expresses a different variant of the epitope-comprising fragment, compared to the variant of target microorganism or epitope-comprising fragment thereof that the donor animal had been exposed to.

In some embodiments, the antibody-producing cells are incubated or contacted with the target microorganism or epitope-comprising fragment thereof, before being introduced into the immunocompromised animal. In some embodiments, the incubation allows or results in the formation of a complex between the antibody-producing cell and the target microorganism or epitope-comprising fragment thereof, by virtue of the recognition of the target epitope by the specific antibodies produced from the antibody-producing cell. In some embodiments, this incubation provides specific stimulation to the candidate cells that produce the antibody of interest, e.g., antibody that binds to the target microorganism or epitope-comprising fragment thereof, in particular a conserved epitope on the target microorganism or epitope-comprising fragment thereof. Thus, this can preferentially stimulate the rare antibody-producing cell of interest, and result in expansion and enrichment of the rare cell of interest.

In some embodiments, the antibody producing cells and/or antigen and/or target microorganisms are introduced into the spleen of the immunocompromised animal or introduced intravenously.

In some embodiments, the antibody-producing cells are from a donor exposed to a first variant of the target microorganism or epitope-comprising fragment thereof, and prior to introducing the cell composition comprising the antibody-producing cells into the immunocompromised animal, the method comprises mixing or incubating the antibody-producing cells with a second variant of the target microorganism or epitope-comprising fragment thereof, wherein the introduced cell composition comprising the antibody-producing cells complexed with the second variant of the target microorganism or epitope-comprising fragment thereof.

In some embodiments, the first and second variant each independently comprises an epitope-comprising fragment of the target microorganism. In some embodiments, the first and the second variant shares at least one conserved region or domain. In some embodiments, the first and the second variant each comprise at least one region or domain that differs from each other, such as a domain or a region that is variable or hypervariable.

In some embodiments, the first and second variant comprises an OM protein or fragment thereof derived from two different clinical isolates of the same microorganism. In some embodiments, the first or second variants can be further modified from existing variants, e.g., clinical isolates. For example, in some embodiments, the first variant and/or second variant is a full-length OM protein and the other of the first and/or second variant is a fragment of the OM protein comprising deletion of an immunodominant epitope or loop of the OM protein.

In some embodiments, the variant of target microorganism or epitope-comprising fragment thereof that the donor animal had been exposed to, e.g., by immunization or infection, is a different from the variant of target microorganism or epitope-comprising fragment thereof used in the stimulation prior to the introduction to the immunocompromised animal. For example, in some embodiments, the donor animal is immunized with one variant of an epitope-comprising fragment from a target microorganism, e.g., BamA variant 1 (set forth in SEQ ID NO:1), and the cell composition obtained from the donor animal is contacted with a different variant of the epitope-comprising fragment from a target microorganism, e.g., BamA variant 2 (set forth in SEQ ID NO:2), prior to introduction into the immunocompromised animal for in vivo enrichment. In some embodiments, the donor animal has been infected with a target microorganism expressing one variant of an epitope-comprising fragment, e.g., BamA variant 1 (set forth in SEQ ID NO:1), and the cell composition obtained from the donor animal is contacted with a different variant of the epitope-comprising fragment from a target microorganism, e.g., BamA variant 2 (set forth in SEQ ID NO:2), prior to introduction into the immunocompromised animal for in vivo enrichment. In some embodiments, BamA variant 3 (set forth in SEQ ID NO:5) and/or BamA variant 4 (set forth in SEQ ID NO:6) may be used in either steps. In some embodiments, any known variants or clinical isolates of BamA can be used for immunization and/or in vivo enrichment.

In some embodiments, any one or more other variants, any other variants of the corresponding epitope-binding fragment from other variants of the target microorganisms, e.g., different clinical isolates or different serotypes, or corresponding epitope-binding fragment from a related but different microorganism, can be used for exposure in the donor animal and stimulation of the antibody-producing cells prior to in vivo enrichment, in any order and/or in any combination. For example, in some embodiments, the variant epitope-comprising fragment comprises a BamA variant with the sequence of amino acids set forth in SEQ ID NO: 1, 2, 5, 6 or 31 or a fragment, region or domain thereof. In some embodiments, the variant epitope-comprising fragment comprises a sequence of amino acids comprising at least 90% sequence identity to sequence of amino acids set forth in SEQ ID NO: 1, 2, 5, 6 or 31 or a fragment, region or domain thereof, such as at least 90%, 91%, 92%, 93%, 94%, 95%, 96%, 97%, 98%, or 99% sequence identity thereto.

Utilizing different variants of the epitope-comprising fragments for donor exposure and in vivo enrichment allows specific stimulation of cells that produce antibodies that target an epitope shared by the different variants used. Thus, this can result in the identification of antibodies targeting conserved epitopes that can be effective against broad range of target microorganism variants, e.g., target microorganisms of different serotypes, or target microorganism species.

In some embodiments, the epitope-comprising fragment is generated and prepared for contacting and/or incubation with the candidate antibody-producing cells in the in vivo rare cell enrichment step. In some embodiments, one or more detergent or surfactant is used to prepare the epitope-comprising fragment, for solubilization and/or refolding of the protein. In particular, for membrane proteins, solubilization and/or refolding steps can be required. In some embodiments, epitope-comprising fragments can be solubilized, denatured and/or refolded using detergents or surfactants in the preparation. In some embodiments, the solubilized and/or denatured preparations can be refolded or re-natured, e.g., in the presence of detergents or surfactants. In some embodiments, the detergent or surfactant is selected from among lauryldimethylamine oxide (LDAO), 2-methyl-2,4-pentanediol (MPD), an amphipol, amphipol A8-35, C8E4, Triton X-100, octylglucoside, DM (n-Decyl-β-D-maltopyranoside), DDM (n-Dodecyl-β-D-maltopyranoside, 3-[(3-Cholamidopropyl)dimethylammonio]-1-propanesulfonate (CHAPS) and 3-[(3-cholamidopropyl)dimethylammonio]-2-hydroxy-1-propanesulfonate (CHAPSO).

In some embodiments, the preparation is subject to a detergent exchange, replacing some or all of the detergent and/or surfactant in the preparation with an amphipathic polymer or a surfactant, such as an amphipol, e.g., amphipol A8-35. In some embodiments, prior to contacting the preparation of epitope-comprising fragments with antibody-producing cells, excess detergent or surfactant is removed or reduced from the preparation of the epitope-comprising fragment to a level or amount that is not toxic to and/or does not induce lysis of the antibody-producing cells. In some embodiments, removal of detergent is carried out using gel filtration columns.

In some embodiments, the methods can include isolating candidate antibody-producing cells, e.g., B cells and/or plasmablasts, from the spleen of the immunocompromised animal, thereby obtaining a plasmablast population enriched for plasmablasts having specificity to an epitope-comprising fragment in the microorganism. In some embodiments, such candidate cells can be subject to encapsulation and identification using any of the methods provided herein.

### IV. Antibodies

Provided are antibodies that bind an epitope-comprising fragment, e.g., an antigen or an epitope, of a target microorganism. In some embodiments, provided are antibodies that bind a bacterial outer membrane (OM) protein. In some embodiments, provided are antibodies that bind Acinetobacter baummannii BamA. In some embodiments, the provided antibodies bind to an epitope in the target microorganism. In some embodiments, provided are antibodies that bind to an epitope present in at least one conserved region of a target microorganism or epitope-comprising fragment thereof, i.e., regions that are conserved between different variants of the microorganism or epitope-comprising fragment thereof, e.g., an antigen or an epitope. In some embodiments, the antibodies are antibodies identified using the methods provided herein.

In some embodiments, provided are antibodies that bind to an epitope present in at least one conserved region of an OM protein of Gram negative bacteria. Provided are antibodies or antigen-binding fragments thereof that bind to an epitope present in at least one conserved region or domain of a Gram-negative bacterium. In some embodiments, provided are antibodies that bind to an epitope present in at least one conserved region of BamA in Acinetobacter species. In some embodiments, provided are antibodies that bind to an epitope present in at least one conserved region, e.g., one or more conserved amino acids that are conserved in one or more variants or isolates, of Acinetobacter baummannii BamA. In some embodiments, provided are antibodies that bind to region that is conserved between BamA from A. baumannii ATCC 19606 and A. baumannii ATCC 17978. In some embodiments, the antibodies bind to a region that is conserved between BamA from A. baumannii strain 1440422, A. baumannii strain MSP4-16 and/or A. baumannii strain 1202252.

In some embodiments, the epitope is or comprises a contiguous sequence of amino acids. In some embodiments, the epitope is or comprises a non-contiguous sequence of amino acids. Exemplary regions that are conserved in various A. baumannii can include amino acid residues 423-438, 440-460, 462-502, 504-533, 537-544, 547-555, 557-561, 599-604, 606-644, 646-652, 659-700, 702-707, 718-723, 735-747, 749-760, 784-794, 798-804, 806-815 and 817-841 of the A. baumannii ATCC 19606 BamA sequence set forth in SEQ ID NO:11. In some embodiments, the conserve regions that are conserved in various A. baumannii include any one or more of the amino acid sequences set forth in SEQ ID NOS:12-30 or any fragments thereof. In some embodiments, the provided antibodies bind to an epitope that is partially or fully contained within the conserved regions.

The antibodies include isolated antibodies. In some embodiments, the provided antibodies are human antibodies. In some embodiments, the provided antibodies are humanized antibodies, such as an antibody in which all or substantially all complementary determining region (CDR) amino acid residues are derived from non-human CDRs and all or substantially all framework region (FR) amino acid residues are derived from human FRs. In some embodiments, the antibodies are monoclonal antibodies. In some embodiments, the antibodies are produced by cells a humanized animal, e.g., an animal genetically engineered to produce humanized antibodies. In some embodiments, the antibodies are produced by cells from a transgenic mouse or a transgenic chicken engineered to produce humanized or partially humanized antibodies, such as the Trianni transgenic mouse, and transgenic chicken, such as the HuMab Chicken from Crystal Biosciences.

In some embodiments, the provided antibodies are capable of binding the epitope-comprising fragment of a target microorganism, with at least a certain affinity, as measured by any of a number of known methods. In some embodiments, the affinity is represented by an equilibrium dissociation constant (K_{D}). In some embodiments, the provided antibodies bind, such as specifically bind, to the epitope-comprising fragment of a target microorganism or an epitope therein, with an affinity or K_{A} (i.e., an equilibrium association constant of a particular binding interaction with units of 1/M; equal to the ratio of the on-rate [kₒₙ or kₐ] to the off-rate [k_{off} or k_{d}] for this association reaction, assuming bimolecular interaction) equal to or greater than 10⁵ M⁻¹. In some embodiments, the provided antibodies bind, such as specifically bind, to the epitope-comprising fragment of a target microorganism or an epitope therein, with a K_{D} (i.e., an equilibrium dissociation constant of a particular binding interaction with units of M; equal to the ratio of the off-rate [k_{off} or k_{d}] to the on-rate [kₒₙ or kₐ] for this association reaction, assuming bimolecular interaction) of equal to or less than 10⁻⁵ M. For example, the equilibrium dissociation constant K_{D} ranges from 10⁻⁵ M to 10⁻¹³ M, such as 10⁻⁷ M to 10⁻¹¹ M, 10⁻⁸ M to 10⁻¹⁰ M, or 10⁻⁹ M to 10⁻¹⁰ M. In certain embodiments, the K_{D}, of the antibody to a The epitope-comprising fragment of a target microorganism, is at or less than or about 400 nM, 300 nM, 200 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM or less.

In some embodiments, the provided antibodies are recombinantly produced. In some embodiments, a polynucleotide comprising the sequence of the nucleic acids encoding the identified antibody or fragment thereof into a cell. In some embodiments, the antibodies are produced in mammalian cell or a cell for recombinant expression, e.g., into bacterial cells or yeast cells. In some embodiments, the polynucleotide includes sequences that are operably linked to polynucleotides encoding another moiety, e.g., an affinity tag, a detectable label, protease cleavage sequence and/or a flexible linker. In some embodiments, the polynucleotide encode a fusion protein of the provided antibody or fragment thereof, and another moiety, e.g., an affinity tag, a detectable label and/or protease cleavage sequence. In some embodiments, the detectable label is a fluorescent protein, a luminescent protein, a chromoprotein or an enzyme.

In some embodiments, the provided antibodies are functional antigen-binding fragments. In some embodiments, the antibodies include those that are single domain antibodies, containing a heavy chain variable (V_{H}) region that, without pairing with a light chain antigen-binding site (e.g., light chain variable (V_{L}) region) and/or without any additional antibody domain or binding site, are capable of specifically binding to the epitope-comprising fragment of a target microorganism or an epitope therein. Also among the antibodies are multi-domain antibodies, such as those containing V_{H} and V_{L} domains, comprised of the V_{H} domain or antigen-binding site thereof of the single-domain antibody. In some embodiments, the antibodies include a heavy chain variable region and a light chain variable region, such as scFvs. The antibodies include antibodies that specifically bind to the epitope-comprising fragment of a target microorganism or an epitope therein.

In certain embodiments, the antibody is altered to increase or decrease the extent to which the antibody is glycosylated, for example, by removing or inserting one or more glycosylation sites by altering the amino acid sequence and/or by modifying the oligosaccharide(s) attached to the glycosylation sites, e.g., using certain cell lines. In some embodiments, an N-linked glycosylation, which is a glycosylation site that occurs at asparagines in the consensus sequence -Asn-Xaa-Ser/Thr is removed or inserted.

For example, in some embodiments, the provided antibodies have one or more amino acid modifications in the Fc region, such as those having a human Fc region sequence or other portion of a constant region (e.g., a human IgG1, IgG2, IgG3 or IgG4 Fc region) comprising an amino acid modification (e.g. a substitution) at one or more amino acid positions. Such modifications can be made, e.g., to improve half-life, alter binding to one or more types of Fc receptors, and/or alter effector functions. Other modifications include cysteine engineering, in which one or more residues of an antibody are substituted with cysteine residues, in order to generate reactive thiol groups at accessible sites, e.g., for use in conjugation of agents and linker-agents, to produce immunoconjugates. Cysteine engineered antibodies are described, e.g., in U.S. Pat. Nos. 7,855,275 and 7,521,541.

In some embodiments, the antibodies (e.g., antigen-binding fragment) are modified to contain additional nonproteinaceous moieties, including water soluble polymers, such as polyethylene glycol (PEG). The polymer may be of any molecular weight, and may be branched or unbranched. The number of polymers attached to the antibody may vary and one or more different polymers can be attached.

### V. Exemplary Embodiments

Illustrative embodiments of these and other aspects of the invention are described in further detail below. However, the invention is not limited to these specific embodiments.

1. A method for identifying an antibody that binds a target microorganism, comprising:

(a) obtaining a plurality of candidate antibody-producing cells;

(b) encapsulating the plurality of candidate antibody-producing cells in gel microdroplets with a target microorganism; and

(c) determining whether the antibody-producing cell(s) within the gel microdroplet produce an antibody that binds the target microorganism, thereby identifying an antibody that specifically binds to the target microorganism.

2. The method of embodiment 1, wherein:

step (b) further comprises encapsulating, in the microdroplets, an epitope-comprising fragment of the target microorganism or a variant thereof; and

step (c) comprises determining whether the antibody identified as binding the target microorganism also binds the epitope-comprising fragment thereof within the same gel microdroplet.

3. A method for identifying an antibody that binds a target microorganism, comprising:

(a) obtaining a plurality of candidate antibody-producing cells;

(b) encapsulating the plurality of candidate antibody-producing cells in gel microdroplets with a target microorganism and with an epitope-comprising fragment of the target microorganism or a variant thereof; and

(c) determining whether the antibody-producing cell(s) within the gel microdroplet produce an antibody that binds the target microorganism and/or epitope-comprising fragment thereof present in the same gel microdroplet, thereby identifying an antibody that specifically binds to the target microorganism or epitope-comprising fragment thereof.

4. The method of any of embodiments 1-3, wherein the epitope-comprising fragment is bound to a solid support.

5. The method of embodiment 4, wherein the solid support is a bead.

6. The method of any of embodiments 1-5, wherein the target microorganism is a bacterium, a fungus, a parasite or a virus.

7. The method of embodiment 6, wherein the target microorganism is a bacterium or a fungus.

8. The method of embodiment 6 or embodiment 7, wherein the microorganism is a multi-drug resistant microorganism.

9. The method of any of embodiments 6-8, wherein the microorganism is a bacterium that is a Gram-negative bacterium.

10. The method of embodiment 9, wherein the Gram-negative bacterium is a proteobacterium.

11. The method of any of embodiments 6-10, wherein the microorganism is a bacterium selected from among a species of Acinetobacter, Bdellovibrio, Burkholderia, Chlamydia, Enterobacter, Escherichia, Francisella, Haemophilus, Helicobacter, Klebsiella, Legionella, Moraxella, Neisseria, Pantoea, Pseudomonas, Salmonella, Shigella, Stenotrophomonas, Vibrio and Yersinia.

12. The method of any of embodiments 6-11, wherein the microorganism is selected from among Acinetobacter apis, Acinetobacter baumannii, Acinetobacter baylyi, Acinetobacter beijerinckii, Acinetobacter bereziniae, Acinetobacter bohemicus, Acinetobacter boissieri, Acinetobacter bouvetii, Acinetobacter brisouii, Acinetobacter calcoaceticus, Acinetobacter gandensis, Acinetobacter gerneri, Acinetobacter guangdongensis, Acinetobacter guillouiae, Acinetobacter gyllenbergii, Acinetobacter haemolyticus, Acinetobacter harbinensis, Acinetobacter indicus, Acinetobacter johnsonii, Acinetobacter junii, Acinetobacter kookii, Acinetobacter lwoffii, Acinetobacter nectaris, Acinetobacter nosocomialis, Acinetobacter pakistanensis, Acinetobacter parvus, Acinetobacter pitii, Acinetobacter pittii, Acinetobacter puyangensis, Acinetobacter qingfengensis, Acinetobacter radioresistans, Acinetobacter radioresistens, Acinetobacter rudis, Acinetobacter schindleri, Acinetobacter seifertii, Acinetobacter soli, Acinetobacter tandoii, Acinetobacter tjernbergiae, Acinetobacter towneri, Acinetobacter ursingii, Acinetobacter variabilis, Acinetobacter venetianus, Escherichia coli, Haemophilus influenzae, Klebsiella pneumoniae, Pseudomonas aeruginosa, Salmonella typhimurium, Shigella boydii, Shigella dysenteriae, Shigella flexneri, Shigella sonnei, Vibrio cholera and Yersinia pestis.

13. The method of embodiment 12, wherein the microorganism is Acinetobacter baumannii.

14. The method of any of embodiments 6-8, wherein the microorganism is a bacterium that is a Gram-positive bacterium.

15. The method of embodiment 14, wherein the microorganism is selected from among a species of Staphylococcus and Streptococcus.

16. The method of any of embodiments 6-8, wherein the microorganism is a fungus that is an Aspergillus species or a Candida species.

17. The method of embodiment 6 or embodiment 8, wherein the microorganism is a parasite that is a Coccidia or a Plasmodium species.

18. The method of any of embodiments 1-17, wherein the plurality of candidate antibody-producing cells are obtained from a donor that has been exposed to the target microorganism or an epitope-comprising fragment of the target microorganism or a variant thereof.

19. The method of any of embodiments 1-18, wherein the plurality of candidate antibody-producing cells is obtained by a method comprising:

(i) expanding antibody-producing cells obtained from a donor that has been exposed to the target microorganism or an epitope-comprising fragment of the target microorganism or a variant thereof by introducing a cell composition comprising the antibody-producing cells into an immunocompromised animal; and

(ii) recovering the expanded antibody-producing cells, thereby obtaining the plurality of candidate antibody-producing cells.

20. The method of embodiment 19, wherein the cell composition comprising the antibody-producing cells comprises cells obtained from the spleen and/or lymph node of the donor.

21. The method of embodiment 19 or embodiment 20, wherein the cell composition comprises T cells.

22. The method of any of embodiments 19-21, wherein the cell composition comprises peripheral blood mononuclear cells (PBMCs) comprising the antibody-producing cells.

23. The method of any of embodiments 19-22, wherein the immunocompromised animal is a SCID mouse.

24. The method of any of embodiments 19-23, wherein the cell composition comprising the antibody-producing cells is introduced into the immunocompromised animal intravenously or by transplant into the immunocompromised animal's spleen.

25. The method of any of embodiments 19-24, wherein:

the antibody-producing cells are from a donor exposed to a first variant of the target microorganism or epitope-comprising fragment thereof, and

prior to introducing the cell composition comprising the antibody-producing cells into the immunocompromised animal, the method comprises mixing or incubating the antibody-producing cells with a second variant of the target microorganism or epitope-comprising fragment thereof, wherein the introduced cell composition comprises the antibody-producing cells complexed with the second variant of the target microorganism or epitope-comprising fragment thereof.

26. The method of any of embodiments 1-25, wherein the epitope-comprising fragment comprises an essential protein or fragment of an essential protein of the target microorganism.

27. The method of any of embodiments 1-26, wherein the epitope-comprising fragment comprises a bacterial outer membrane (OM) protein, a membrane protein, an envelope proteins, a cell wall protein, a cell wall component, a surface lipid, a glycolipid, a lipopolysaccharide, a glycoprotein, a surface polysaccharide, a capsule, a surface appendage, a flagellum, a pilus, a monomolecular surface layer, or an S-layer or a fragment thereof derived from the target microorganism.

28. The method of any of embodiments 1-27, wherein the epitope-comprising fragment comprises a lipid from the surface of the target microorganism.

29. The method of embodiment 28, wherein the epitope-comprising fragment comprises a lipopolysaccharide (LPS) or a lipoprotein.

30. The method of any of embodiments 1-27, wherein the epitope-comprising fragment comprises an outer membrane (OM) protein.

31. The method of embodiment 30, wherein the OM protein is selected from among BamA, LptD, AdeC, AdeK, BtuB, FadL, FecA, FepA, FhaC, FhuA, LamB, MepC, MexA, NalP, NmpC, NspA, NupA, Omp117, Omp121, Omp200, Omp71, OmpA, OmpC, OmpF, OmpG, OmpT, OmpW, OpcA, OprA, OprB, OprF, OprJ, OprM, OprN, OstA, PagL, PagP, PhoE, PldA, PorA, PorB, PorD, PorP, SmeC, SmeF, SrpC, SucY, TolC, TtgC and TtgF.

32. The method of embodiment 31, wherein the OM protein is BamA or LptD.

33. The method of any of embodiments 25-27 and 30-32, wherein the epitope-comprising fragment is prepared by solubilization of the OM protein or a fragment thereof.

34. The method of embodiment 33, wherein solubilization is carried out by addition of one or more detergent or surfactant.

35. The method of embodiments 33 or embodiment 34, further comprising refolding of the epitope-comprising fragment prior to mixing or incubating with the antibody-producing cells.

36. The method of embodiment 35, wherein the refolding is carried out in the presence of one or more detergent or surfactant.

37. The method of any of embodiments 34-36, wherein the detergent or surfactant is selected from among lauryldimethylamine oxide (LDAO), 2-methyl-2,4-pentanediol (MPD), an amphipol, amphipol A8-35, C8E4, Triton X-100, octylglucoside, DM (n-Decyl-β-D-maltopyranoside), DDM (n-Dodecyl-β-D-maltopyranoside, 3-[(3-Cholamidopropyl)dimethylammonio]-1-propanesulfonate (CHAPS) and 3-[(3-cholamidopropyl)dimethylammonio]-2-hydroxy-1-propanesulfonate (CHAPSO).

38. The method of any of embodiments 34-37, further comprising replacing some or all of the detergent and/or surfactant in the preparation with an amphipathic polymer or a surfactant.

39. The method of any of embodiments 34-38, wherein prior to mixing or incubating with the antibody-producing cells, excess detergent or surfactant is removed or reduced from the preparation of the epitope-comprising fragment to a level or amount that is not toxic to and/or does not induce lysis of the antibody-producing cells.

40. The method of any of embodiments 25-39, wherein the first and second variant each independently comprises an epitope-comprising fragment of the target microorganism.

41. The method of any of embodiments 25-40, wherein the first and the second variant shares at least one conserved region or domain.

42. The method of embodiment 41, wherein the first and the second variant each comprise at least one region or domain that differs from each other.

43. The method of any of embodiments 25-42, wherein the first and second variant comprises an OM protein or fragment thereof derived from two different clinical isolates of the same microorganism.

44. The method of any of embodiments 25-43, wherein the first variant and/or second variant is a full-length OM protein and the other of the first and/or second variant is a fragment of the OM protein comprising deletion of an immunodominant epitope or loop of the OM protein.

45. The method of any of embodiments 41-44, wherein the identified antibody binds to the at least one conserved region or domain of the target microorganism.

46. The method of any of embodiments 18-45, wherein the donor has been immunized or infected with the target microorganism or an epitope-comprising fragment of the target microorganism or a variant thereof.

47. The method of any of embodiments 18-46, wherein the donor is an immunized animal or an infected animal.

48. The method of any of embodiments 18-47, wherein the donor is a mammal or a bird.

49. The method of any of embodiments 18-48, wherein the donor is a human, a mouse or a chicken.

50. The method of any of embodiments 18-49, wherein the donor is a human donor who was infected by the microorganism.

51. The method of any of embodiments 18-50, wherein the donor is a genetically modified non-human animal that produces partially human or fully human antibodies.

52. The method of any of embodiments 1-51, wherein the antibody-producing cells comprise peripheral blood mononuclear cells (PBMCs), B cells, plasmablasts or plasma cells.

53. The method of any of embodiments 1-52, wherein the antibody-producing cells comprise B cells, plasmablasts or plasma cells.

54. The method of any of embodiments 18-53, wherein the plurality of candidate antibody-producing cells are selected from the donor by a positive or negative selection to isolate or enrich for B cells.

55. The method of embodiment 54, wherein the B cell is a plasmablast or a plasma cell.

56. The method of embodiment 55, wherein the selection is a positive selection based on expression of a cell surface marker selected from among one or more of: CD2, CD3, CD4, CD14, CD15, CD16, CD34, CD56, CD61, CD138, CD235a (Glycophorin A) and FceRIa.

57. The method of any of embodiments 52-56, wherein the antibody-producing cells comprise CD138+ cells.

58. The method of any of embodiments 52-57, wherein at least or at least about 50%, 60%, 70%, 80%, 85%, 90%, 95%, or more of the cells are plasma cells or plasmablasts and/or are CD138+ cells.

59. The method of any of embodiments 1-58, wherein the antibody is an antibody or an antigen-binding fragment thereof.

60. The method of any of embodiments 1-59, wherein the gel microdroplet is generated by a microfluidics-based method.

61. The method of any of embodiments 1-60, wherein the gel microdroplet comprises material selected from among agarose, carrageenan, alginate, alginate-polylysine, collagen, cellulose, methylcellulose, gelatin, chitosan, extracellular matrix, dextran, starch, inulin, heparin, hyaluronan, fibrin, polyvinyl alcohol, poly(N-vinyl-2-pyrrolidone), polyethylene glycol, poly(hydroxyethyl methacrylate), acrylate polymers and sodium polyacrylate, polydimethyl siloxane, cis-polyisoprene, Puramatrix™, poly-divenylbenzene, polyurethane, or polyacrylamide or combinations thereof.

62. The method of embodiment 61, wherein the gel microdroplet comprises agarose.

63. The method of embodiment 62, wherein the agarose is low gelling temperature agarose.

64. The method of embodiment 62 or embodiment 63, wherein the agarose has a gelling temperature of lower than about 35° C., about 30° C., about 25° C., about 20° C., about 15° C., about 10° C. or about 5° C.

65. The method of embodiment 62 or embodiment 63, wherein the agarose has a gelling temperature of between about 5° C. and about 30° C., about 5° C. and about 20° C., about 5° C. and about 15° C., about 8° C. and about 17° C. or about 5° C. and about 10° C.

66. The method of any of embodiments 1-65, wherein step (b) further comprises incubating the gel microdroplets at a temperature of between about 0° C. and about 5° C. for about 1 minute to about 10 minutes subsequent to encapsulation.

67. The method of any of embodiments 5-66, wherein the bead has an average diameter of between about 100 nm and about 100 μm, or between about 3 μm and about 5 μm.

68. The method of any of embodiments 1-67, wherein the average ratio of candidate antibody-producing cell per gel microdroplet is less than or less than about 1.

69. The method of any of embodiments 1-68, wherein the average ratio of candidate antibody-producing cell per gel microdroplet is between about 0.05 and about 1.0, about 0.05 and about 0.5, about 0.05 and about 0.25, about 0.05 and about 0.1, about 0.1 and about 1.0, about 0.1 and about 0.5, about 0.1 and about 0.25, about 0.25 and about 1.0, about 0.25 and about 0.5 or 0.5 and about 1.0, each inclusive.

70. The method of embodiment 69, wherein the average ratio of candidate antibody-producing cells per microdroplet is or is about 0.1.

71. The method of any of embodiments 1-70, wherein the average ratio of the microorganism per gel microdroplet is between about 50 and about 150 or about 50 and about 100.

72. The method of any of embodiments 5-71, wherein the average ratio of the bead per gel microdroplet is between about 2 and about 10 or about 3 and about 5.

73. The method of any of embodiments 5-72, wherein the average ratio of the candidate cell to microorganism to bead is about 0.1:100:10.

74. The method of any of embodiments 1-73, wherein the gel microdroplets comprise growth media and are surrounded by a non-aqueous environment.

75. The method of embodiment 74, wherein the non-aqueous environment comprises an oil.

76. The method of embodiment 75, wherein the oil is gas permeable. 77. The method of any of embodiments 1-76, further comprising incubating the gel microdroplets at a temperature of at or about 37° C. prior to step (c).

78. The method of embodiment 77, wherein the gel microdroplets are incubated in growth media.

79. The method of any of embodiments 1-78, wherein prior to step (c), introducing into the gel microdroplets a reagent that binds to antibodies, said reagent comprising a detectable moiety.

80. The method of embodiment 79, wherein the reagent comprises a secondary antibody specific for antibodies produced by the encapsulated antibody-producing cells.

81. The method of embodiment 79 or embodiment 80, wherein determining whether the antibody-producing cell(s) within the gel microdroplet produce an antibody that binds the target microorganism and/or epitope-comprising fragment thereof present in the same gel microdroplet comprises detecting the presence of a complex comprising: (i) the target microorganism or epitope-comprising fragment thereof; (ii) the antibody produced by the antibody-producing cell; and (iii) the reagent comprising the detectable moiety bound, wherein the presence of the complex indicates that the antibody specifically binds the target microorganism or epitope-comprising fragment thereof.

82. The method of any of embodiments 1-78, wherein determining whether the antibody-producing cell(s) within the gel microdroplet produce an antibody that binds the target microorganism and/or epitope-comprising fragment thereof present in the same gel microdroplet comprises determining whether the presence of the antibody modifies a phenotypic characteristic of the target microorganism in the same gel microdroplet, wherein the presence of the modified phenotypic characteristic indicates that the antibody specifically binds the target microorganism or epitope-comprising fragment thereof.

83. The method of embodiment 82, wherein the modified phenotypic characteristic is selected from among cell growth, cell death, changes in in behavior, binding, transcription, translation, expression, protein transport, cellular or membrane architecture, adhesion, motility, cellular stress, cell division and/or cell viability.

84. The method of embodiment 82 or embodiment 83, wherein determining whether the antibody-producing cell(s) within the gel microdroplet produce an antibody that binds the target microorganism and/or epitope-comprising fragment thereof present in the same gel microdroplet comprises detecting a signal produced by a reporter molecule, wherein the signal is produced in the presence of the modified phenotypic characteristic.

85. The method of embodiment 84, wherein the microorganism comprises a polynucleotide encoding the reporter molecule.

86. The method of embodiment 85, wherein the polynucleotide comprises a regulatory region operably linked to a sequence encoding the reporter molecule, wherein the regulatory region is responsive to the modified phenotypic characteristic.

87. The method of embodiment 86, wherein the regulatory region comprises a promoter.

88. The method of any of embodiments 82-87, wherein the modified phenotypic characteristic comprises cellular stress and the signal is produced in the presence of the cellular stress.

89. The method of any of embodiments 83-88, wherein the cellular stress comprises stress to the outer membrane (OM) of the bacterium.

90. The method of any of embodiments 84-89, wherein the signal produced by the reporter molecule is detected with a detectable moiety.

91. The method of any of embodiments 84-90, wherein the signal produced by the reporter molecule comprises a fluorescent signal, a luminescent signal, a colorimetric signal, a chemiluminescent signal or a radioactive signal.

92. The method of any of embodiments 84-91, wherein the reporter molecule is a fluorescent protein, a luminescent protein, a chromoprotein or an enzyme.

93. The method of any of embodiments 1-78, wherein determining whether the antibody-producing cell(s) within the gel microdroplet produce an antibody that binds the target microorganism and/or epitope-comprising fragment thereof present in the same gel microdroplet comprises determining whether the presence of the antibody kills the target microorganism in the same gel microdroplet, wherein killing of the target microorganism indicates that the antibody specifically binds the target microorganism or epitope-comprising fragment thereof.

94. The method of embodiment 93, wherein the gel microdroplets comprise a detectable moiety indicative of cell death.

95. The method of any of embodiments 79-81, 90-92 and 94, wherein the detectable moiety comprises one or more detectable label selected from among a chromophore moiety, a fluorescent moiety, a phosphorescent moiety, a luminescent moiety, a light absorbing moiety, a radioactive moiety, and a transition metal isotope mass tag moiety.

96. The methods of any of embodiments 1-95, further comprising:

(d) isolating the microdroplet comprising the cell producing the identified antibody or isolating polynucleotides encoding the antibody identified as specifically binding the target microorganism or epitope-comprising fragment thereof.

97. The method of embodiment 96, wherein isolation is carried out using a micromanipulator or an automated sorter.

98. The method of any of embodiments 1-97, further comprising:

(e) determining the sequence of the nucleic acids encoding the identified antibody.

99. The method of embodiment 98, wherein determining the sequence of the nucleic acids is carried out using nucleic acid amplification and/or sequencing.

100. The method of embodiment 98 or embodiment 99, wherein determining the sequence of the nucleic acids is carried out using single cell PCR and nucleic acid sequencing.

101. The methods of any of embodiments 98-100, further comprising:

(f) introducing a polynucleotide comprising a sequence of the nucleic acids encoding the identified antibody or fragment thereof into a cell.

102. The method of any of embodiments 1-101, wherein the method is completed within about 60 days, 50 days, 40 days, 30 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days or 1 day from completion of step (a).

103. The method of embodiment 102, wherein the method is completed within about 30 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days or 1 day from completion of step (a).

104. The antibody identified by the method of any of embodiments 1-103, or an antigen-binding fragment thereof.

105. The antibody or antigen-binding fragment thereof of embodiment 104, that binds to an epitope present in the at least one conserved region or domain of BamA (β-barrel assembly machinery) of a Gram-negative bacterium.

106. An antibody or antigen-binding fragment thereof, wherein said antibody or antigen-binding fragment thereof binds to an epitope present in at least one conserved region or domain of BamA (β-barrel assembly machinery) of a Gram-negative bacterium.

107. The antibody or antigen-binding fragment thereof of embodiment 105 or embodiment 106, wherein the Gram negative bacterium is an Acinetobacter species.

108. The antibody or antigen-binding fragment thereof of any of embodiment 105-107, wherein the Gram negative bacterium is Acinetobacter baummannii.

109. The antibody or antigen-binding fragment thereof of any of embodiments 105-108, wherein the conserved region or domain is a conserved region or domain that is shared between BamA from A. baumannii ATCC 19606 and A. baumannii ATCC 17978.

110. The antibody or antigen-binding fragment thereof of embodiment 109, wherein the conserved region or domain comprises amino acid residues 423-438, 440-460, 462-502, 504-533, 537-544, 547-555, 557-561, 599-604, 606-644, 646-652, 659-700, 702-707, 718-723, 735-747, 749-760, 784-794, 798-804, 806-815 and 817-841 A. baumannii BamA sequence set forth in SEQ ID NO:11.

111. The antibody or antigen-binding fragment thereof of embodiment 110, wherein the conserved region or domain comprises the sequences set forth in SEQ ID NOS:12-20.

112. The antibody or antigen-binding fragment thereof of any of embodiments 105-111, wherein the epitope is a contiguous or non-contiguous sequence of the conserved region or domain.

113. The antibody or antigen-binding fragment of any of embodiments 104-112, wherein the antibody or antigen-binding fragment is human.

114. The antibody or antigen-binding fragment of any of embodiments 104-112, wherein the antibody or antigen-binding fragment is a humanized antibody.

115. The antibody or antigen-binding fragment of embodiment 114, wherein the antibody or antigen-binding fragment thereof is produced by antibody-producing cells from a transgenic animal engineered to produce humanized antibodies.

116. The antibody or antigen-binding fragment of any of embodiments 104-115 wherein the antibody or antigen-binding fragment is recombinant.

117. The antibody or antigen-binding fragment of any of embodiments 104-116, wherein the antibody or antigen-binding fragment is monoclonal.

118. The antibody or antigen-binding fragment of any of embodiments 104-117, that is an antigen-binding fragment.

119. The antibody or antigen-binding fragment of any of embodiments 104-118, wherein said antibody or antigen-binding fragment further comprises an affinity tag, a detectable protein, a protease cleavage sequence, a linker or a nonproteinaceous moiety.

120. The antibody or antigen-binding fragment of any of embodiments 104-11911, wherein:

said antibody or antigen-binding fragment has an equilibrium dissociation constant (K_{D}) for A. baumannii BamA of at or less than or less than about 400 nM, 300 nM, 200 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM.

121. A polynucleotide encoding the antibody or antigen-binding fragment thereof of any of embodiments 104-120.

122. A composition comprising the antibody of any of embodiments 104-120.

123. The composition of embodiment 122, further comprising a pharmaceutically acceptable excipient.

124. A composition comprising a plurality of microdroplets, each microdroplet comprising:

a candidate antibody-producing cell; and

a target microorganism.

125. The composition of embodiment 124, wherein each microdroplet further comprises the target microorganism or epitope-comprising fragment thereof or a variant thereof bound to a solid support.

126. The composition of embodiment 124 or embodiment 125, wherein the target microorganism comprises a polynucleotide encoding a reporter molecule.

127. A library of microdroplets, each microdroplet comprising:

a candidate antibody-producing cell; and

a target microorganism.

128. The library of embodiment 127, each microdroplet further comprises the target microorganism or epitope-comprising fragment thereof or a variant thereof bound to a solid support.

129. The library of embodiment 127 or embodiment 128, wherein the target microorganism comprises a polynucleotide encoding a reporter molecule.

130. A method for identifying an antibody that specifically binds to a target pathogen or epitope-comprising fragment thereof, comprising:

(a) expanding antibody-producing cells obtained from an animal infected by or immunized with the target pathogen or epitope-comprising fragment thereof by introducing the antibody-producing cells into an immunocompromised animal;

(b) encapsulating antibody-producing cells obtained from the immunocompromised animal following step (a) in gel micro-droplets together with the target pathogen and/or epitope-comprising fragment thereof, wherein a plurality of the gel micro-droplets comprise only one antibody-producing cell; and

(c) determining whether the antibody-producing cell(s) within the gel micro-droplet produce an antibody that binds the target pathogen and/or epitope-comprising fragment thereof present in the same gel micro-droplet, thereby identifying an antibody that specifically binds to the target pathogen or epitope-comprising fragment thereof.

131. The method of embodiment 130, further comprising isolating polynucleotides encoding the antibody identified as specifically binding the target pathogen or epitope-comprising fragment thereof.

132. The method of embodiment 130 or embodiment 131, wherein the animal infected by or immunized with the target pathogen or epitope-comprising fragment thereof is a human donor who was infected by the pathogen.

133. The method of embodiment 130 or embodiment 131, wherein the animal infected by or immunized with the target pathogen or epitope-comprising fragment thereof is a genetically modified non-human animal that produces partially human or fully human antibodies.

134. The method of any one of embodiments 130-133, wherein the pathogen is a microorganism.

135. The method of embodiment 134, wherein the microorganism is a bacterium or a fungus.

136. The method of any of embodiments 130-135, wherein the immunocompromised animal is a SCID mouse.

137. The method of any of embodiments 130-136, wherein PBMCs comprising the antibody-producing cells are introduced into the immunocompromised animal.

138. The method of any one of embodiments 130-137, wherein the antibody-producing cells are introduced into the immunocompromised animal intravenously or by transplant into the immunocompromised animal's spleen.

139. The method of any one of embodiments 130-138, wherein the gel micro-droplets comprise a detectable moiety that binds to antibodies.

140. The method of embodiment 139, wherein the detectable moiety is a labeled secondary antibody specific for antibodies produced by the encapsulated antibody-producing cells.

141. The method of embodiment 139 or embodiment 140, wherein determining whether the antibody-producing cell(s) within the gel micro-droplet produce an antibody that binds the target pathogen and/or epitope-comprising fragment thereof present in the same gel micro-droplet comprises detecting the presence of a complex comprising: (i) the target pathogen or epitope-comprising fragment thereof; the antibody produced by the antibody-producing cell; and (iii) the detectable moiety, wherein the presence of the complex indicates that the antibody specifically binds the target pathogen or epitope-comprising fragment thereof.

142. The method of any one of embodiments 130-138, wherein determining whether the antibody-producing cell(s) within the gel micro-droplet produce an antibody that binds the target pathogen and/or epitope-comprising fragment thereof present in the same gel micro-droplet comprises determining whether the presence of the antibody modifies a phenotypic characteristic of the target pathogen in the same gel micro-droplet, wherein the presence of the modified phenotypic characteristic indicates that the antibody specifically binds the target pathogen or epitope-comprising fragment thereof.

143. The method of embodiment 142, wherein the modified phenotypic characteristic is cell growth or cell death.

144. The method of any one of embodiments 130-138, wherein determining whether the antibody-producing cell(s) within the gel micro-droplet produce an antibody that binds the target pathogen and/or epitope-comprising fragment thereof present in the same gel micro-droplet comprises determining whether the presence of the antibody kills the target pathogen in the same gel micro-droplet, wherein killing of the target pathogen indicates that the antibody specifically binds the target pathogen or epitope-comprising fragment thereof.

145. The method of embodiment 144, wherein the gel micro-droplets comprise a detectable moiety indicative of cell death.

### VI. Examples

The following examples are included for illustrative purposes only and are not intended to limit the scope of the invention.

### Example 1: Illustrative Methods for Gel Encapsulation and Screening

This example describes an illustrative method of gel encapsulation and screening according to the provided methods.

Live mammalian antibody-producing cells (in this case hybridoma cells, but plasma cells also could be used) were encapsulated in agarose particles (approx. 100 um diameter) along with bacterial cells expressing an antigen of interest, BamA, and with beads conjugated with BamA, an exemplary protein (antigen) of interest. The hybridoma cells were cells that secreted either an antibody known to bind to BamA on the surface of live bacterial cells, or a control antibody, to test whether the encapsulation screening can be used to quickly distinguish cells that produce antibodies that bind to the antigen of interest, and cells that do not.

To generate agarose particles, agarose (stored at 4° C.) was heated to 70° C. in a water bath and then cooled to 37° C. A sample, containing B cells, the bacterial pathogen of interest and the beads conjugated to the exemplary BamA protein, was prepared in a 300 μL volume (in media) and warm to 37° C. for 5 minutes. Approximately 300 μL 4% agarose was added to the sample and mixed well. Slowly, 600 μL dispersed phase was added to 16 mL 200 centistokes (cSt) dimethylpolysiloxane (DMPS) oil at 37° C., and then rapidly stirred for 2 minutes. This was transferred to an ice bath and was slowly stirred for 5 minutes to solidify, to which approximately 15 mL of media was poured on top. The agarose particles were pelleted by centrifugation for 9 minutes at 2600 rpm (˜1800×g). The agarose particles were washed in 15 mL hybridoma media, each sample was resuspended in 30 mL growth media, transferred to a T75 flask and incubated at 37° C. in CO₂ for 3 hours with agitation. The agarose particles were incubated in the growth media to allow the mammalian cell to secrete antibody.

The agarose particles were washed and stained with a fluorescent detection agent (i.e. secondary antibody, live/dead stain). Specifically, the agarose particles were pelleted, washed once in in cold PBS and each sample was resuspended in 2 mL PBS+10% FBS containing 20 μg/ml goat anti-mouse (GAM) (H+L)-AF488 labeled secondary antibody and 2 uM Syto® 64 red fluorescent nucleic acid stain and incubated on ice for 45 minutes. The incubated solution was washed two times in cold PBS, the pellet resuspended in 1 mL cold PBS and store on ice until imaging.

The agarose particles were screened using an inverted fluorescence microscope searching for either bacteria or beads that were fluorescent in the same channel (i.e. green fluorescence). Exemplary images from the encapsulation are shown in FIG. 3. As shown in FIG. 3, agarose particles containing bacterial pathogen cells encapsulated with hybridoma cells, “designated pathogen antibody traps (PAT), that were positive for the fluorescent signal indicated that the encapsulated hybridoma cell secreted an antibody bound to BamA on beads and on the bacterial surface. FIG. 3 also shows, in the larger field image, that antigen positive agarose particles can be readily detected.

‘Hit’ particles are picked and deposited in PCR tubes for single-cell cloning. The resulting DNA from single-cell RT-PCR will be expressed in a TBD transient transfection expression system.

### Example 2: Methods for Rare B Cell Enrichment

This example demonstrates methods for enriching for rare B cells that produce antibodies to highly conserved epitopes, The methods may be used to find rare antibodies to highly conserved epitopes on essential Gram-negative proteins, such as BamA and LptD of Acinetobacter baummannii. In addition, the enriched B cells can be used in provided methods to rapidly discovery antibodies to important and conserved epitopes on any infectious disease target.

A. Acinetobacter baumannii BamA as as a Target

Experiments were performed to assess if BamA is a selected target of interest within A. baummannii for targeting by an antibody-based discovery method. BamA was shown to be essential for A. baumannii survival using BamA protein depletion analysis. The BamA protein is located within the outer-membrane and therefore, is accessible to an antibody. To confirm accessibility, a panel of BamA specific antibodies was generated that could bind to the target on the surface of a clinical isolate of A. baumannii.

To understand the BamA epitopes of highest conservation, protein sequence analysis was performed on over one-hundred A. baumannii clinical isolates. The consensus sequence was assessed and mapped onto a structural model of BamA to illustrate regions of high conservation. Interestingly, while the membrane embedded beta-strands and periplasmic loops were highly conserved across all isolates, a few of the extracellular antibody-accessible loops showed significant diversity (FIG. 4). Specifically, loop 4 seems to be highly variable. Interestingly, the panel of antibodies described above to validate BamA accessibility was shown to bind to the variable loop 4. This panel of antibodies was generated using the traditional hybridoma technology, indicating that antibodies directed at loop 4 dominate the host immune response toward BamA. Loop 4, when deleted from the protein, did not affect function, which is consistent with the general concept that protein regions of low conservation are typically not important for function. To further confirm its effect on function, antibodies that bound to this variable loop were shown not to inhibit BamA function. The low conservation, lack of functional importance, and host immuno-dominance makes loop 4 a classic immune-system decoy.

However, a highly conserved epitope on the antibody-accessible surface of BamA that would be amenable to antibody binding was identified (FIG. 4, e.g. circled region). The presence of a conserved epitope is consistent with the ability of pathogens to often produce highly variable decoy epitopes and to protect highly conserved epitopes of greatest importance. Because BamA changes conformations to perform essential functions, an antibody that binds to this highly conserved epitope could block the function and lead to bacterial cell death.

B. Phase 1: Rare B Cell Enrichment

The studies described above demonstrated that BamA was a good target to enrich for rare antibodies that bind highly conserved epitopes. Two different variants of the A. baumannii BamA protein that differed dramatically at the amino acid level within loop 4 were produced. The variants also were engineered with an N-terminal Avi-10His-TEV tag. BamA-variant 1 was engineered with an N-terminal Avi-10His-TEV tag, and to delete an N-terminal periplasmic domain containing five globular POTRA subdomains in tandem, and the sequence is set forth in SEQ ID NO:3. The N-terminal deletion was intended to help bias the B cell enrichment toward B cells that made antibodies to conserved epitopes on the surface of the bacteria. BamA-variant 2 was engineered with an N-terminal 6×His tag, and the sequence is set forth in SEQ ID NO:4. The engineered BamA-variant 2 retained the N-terminal periplasmic domain.

To generate the starting antibody-producing cell pool, BALB/c mice were immunized with BamA-variant 1. It was shown that the vast majority of antibodies produced bound to loop 4. Next, those B cells were subjected to the rare B cell enrichment phase. B cells were harvested from the lymph nodes of the immunized mice and mixed with the BamA-variant 2 to provide a survival signal to only those B cells that have surface antibody that recognizes conserved BamA epitopes. Therefore, all B cells that make a loop 4 antibody should be depleted from the population because loop 4 is not conserved between the original immunogen (BamA-variant 1) and the expansion antigen (BamA-variant 2). Because solubilization is required to purify BamA from the membrane when it is recombinant expressed, experiments also were performed to remove detergent prior to mixing BamA with the B cells, since the presence of detergent impacted the viability of the B cells. Example 6 below describes an exemplary method to remove detergent.

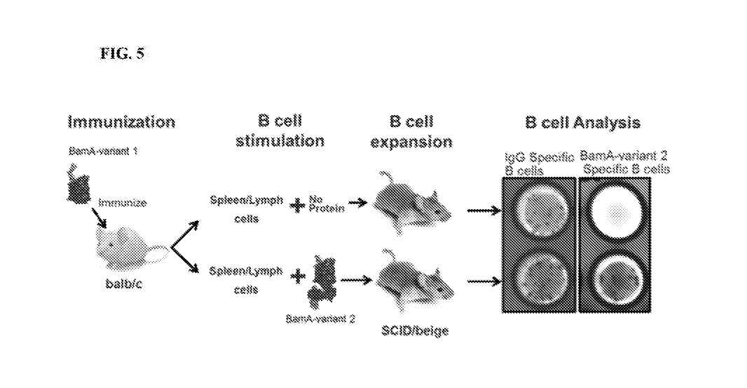

After BamA-variant 2 stimulation, the B cells were injected into the spleens of recently irradiated SCID/beige mice, where they were allowed to propagate for 10 days. As a control, half of the starting B cell population, which was not stimulated with BamA-variant 2, also was injected into the SCID spleen without antigen stimulation. After the 10 day antigen specific expansion in the SCID mouse, the B cells were harvested and subjected to ELISpot analysis to determine if the B cells that received BamA-variant 2 stimulation had a higher frequency of antibodies to conserved epitopes. The BamA-variant 2 stimulation generated a significant number of B cells making antibodies to conserved epitopes, while the mock treated cells showed no cross-reactivity to BamA-variant 2 by ELISpot (FIG. 5).

### Example 3: Pathogen Antibody Trap (PAT) for Screening Antibodies

This example demonstrates implementation of the provided methods to screen large numbers of B cells for binding to a target of interest and isolation of the antibodies by single B cell cloning and transfection protocols. This method can be used to rapidly identify antibodies against a bacteria or other microorganism during an outbreak.

A. Functional Antibody Selection

Antibody secreting B cells from a BALB/c mouse immunized with BamA-variant 1 (set forth in SEQ ID NO:3) were screened. Single B cells were co-encapsulated with bacterial cells expressing BamA-variant 1 and with beads coated with BamA-variant 2 (set forth in SEQ ID NO: 4). In some experiments, the antibody-secreting cells, the beads and the cells were encapsulated generally as described in Example 4 below. These B cells were then allowed to secrete primary antibody into the particle.

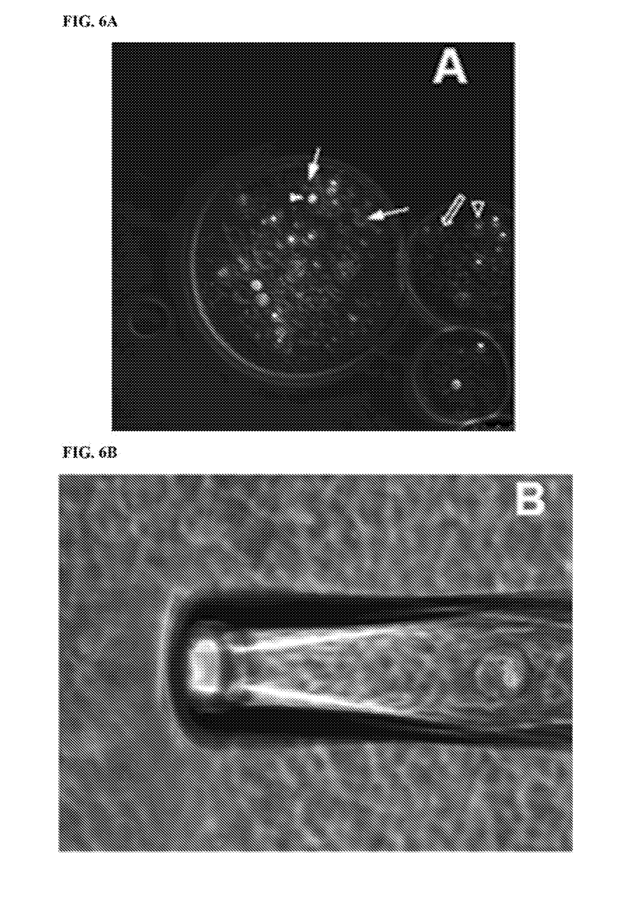

To visualize primary antibody binding to the bacteria or beads, a fluorescent secondary antibody was soaked into the particles. Various binding patterns were observed. FIG. 6 illustrates a representative study. As shown in FIG. 6, B cells that produced antibodies to a conserved surface-exposed epitope on BamA were identified by the presence of fluorescent beads and fluorescent bacteria. Other particles exhibited fluorescent signal on bacteria or beads only, identifying antibodies that bind the surface-exposed variable regions or non-surface-exposed conserved regions of BamA, respectively. These different binding patterns permitted assessment of the correlation between the binding patterns observed in the encapsulated particles to that of antibody binding of the final recombinant antibodies.

In a first screen, 20,000 antibody secreting B cells were screened in one day and ten particles with fluorescent signal on BamA-variant 2 coated beads were identified. On a second day, 50,000 antibody secreting B cells were screened and two particles with fluorescent bacteria and beads (FIG. 6) and fourteen particles with bacterial fluorescent signal were selected. These selected particles were selected for single B cell cloning.

B. Single B Cell Cloning

The particles selected above were then processed for single cell reverse transcription (RT) and PCR of the heavy and light chain genes that encode the antibody. Of the ten B cells expressing antibodies with an epitope to a conserved region of BamA, PCR products for both heavy and light chain genes from six of the B cells were generated.

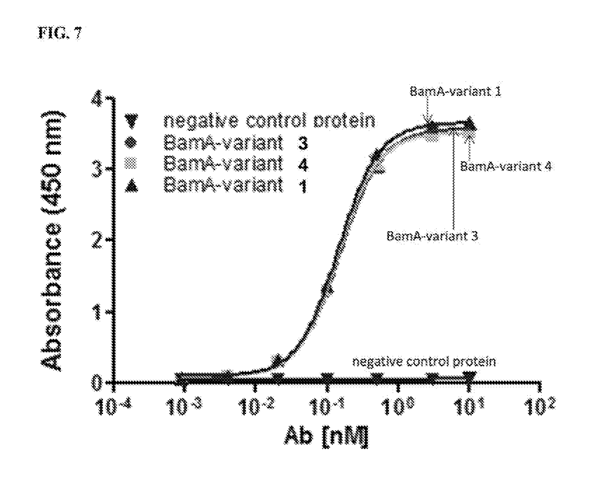

The linear PCR products that encoded the antibodies were transfected into mammalian cells to produce recombinant antibody that was tested for BamA binding by ELISA, for binding to a preparation of BamA variant 1 (set forth in SEQ ID NO:3), BamA variant 3 (set forth in SEQ ID NO:7) and BamA variant 4 (set forth in SEQ ID NO:8): Five of these recombinant antibodies bound highly conserved epitopes on BamA (FIG. 7). As shown in FIG. 7, antibody binding was confirmed for these antibodies by ELISA on three different BamA variants (BamA variants 1, 3 and 4) that all differed dramatically in the sequence of loop 4 and other variable extracellular loops.

### Example 4: Microorganism Encapsulation and Particle Screening

This Examples describes an exemplary method to prepare an antibody-producing cell sample, encapsulate the antibody-producing cells with microorganisms and/or beads and screen particles to rapidly and efficiently identify cells producing antibodies against an immunogen of interest, such as BamA or other bacterial outer membrane protein or immunogen.

A. Preparation of Antibody-Producing Cells

Mice, e.g. balb/C mice, were immunized with an immunogen of interest, such as with A. baumannii bacterial cells or a purified BamA protein or variant thereof. Several weeks later, spleens and/or lymph nodes from immunized mice were removed. Antibody-producing cells were isolated using the Pan B Cell Isolation Kit (Miltenyi Biotec, Cat. No. 130-095-813) followed by CD138+ cell isolation using the EasySep™ CD138+ cell isolation kit (Stemcell Technologies). The pan B cell preparation contained approximately 10% antibody-producing cells and also resulted in a cell preparation devoid of tissue debris and unknown junk. The further CD138+ cell isolation further enriched for the antibody-producing cells by more than 3-fold. The high enrichment of antibody-producing cells during this step can increase the efficiency of the screen by reducing the number of total particles screened to find a particle of interest (hit), which is advantageous because cell viability during the screen can, in some cases, decrease with increasing time of the screen.

B. Encapsulation

The isolated cells containing antibody-producing cells were encapsulated with beads (average diameter: 3-5 μm) coated with an immunogen or target of interest (e.g. such as BamA or other outer membrane protein described in Example 4) and a microorganism to be screened (e.g. A. baumannii bacterial cells) (average size: 0.5-1 μm).

Prior to encapsulation, a preparation of 4% ultra low gelling temperature agarose (Sigma-Aldrich, Cat. No. A5030) in phosphate buffered saline (PBS) was melted at 70° C. for 15 minutes, then cooled to 37° C. for encapsulation of live antibody-producing cells. Ultra-low gelling temperature agarose allows the agarose to stay in liquid state at much lower temperature, thereby permitting encapsulation of live cells.

The individual components to be encapsulated were spun down and resuspended to a desired concentration for encapsulation. The isolated cells containing antibody-producing cells were centrifuged and resuspended in encapsulation media (combination of Iscove's Modified Dulbecco's Medium (IMDM) and OptiPrep™ Density Gradient Medium (Sigma-Aldrich, Cat. No. D1556)). The density gradient media was included to prevent sedimentation of the antibody-producing cell during encapsulation, which can increase efficiency of single cell encapsulation in the particles. Antigen-coated beads and bacterial cells were centrifuged and resuspended in 2× OptiPrep™ Density Gradient Medium. The antibody-producing cells, beads and bacterial cells were combined at an approximate ratio of 0.1:5:100, per agarose gel particle. The ratio was determined based on optimization of cell viability and potential fluorescent signal for each component per agarose gel particle. In some cases, it was found that too many beads per particle resulted in clumped beads that trapped fluorescent antibody and emitted non-specific fluorescent signal. Further, in some aspects, less than 3 beads per particle was found to decrease fluorescent signal, which, in some cases, made the beads more difficult to visualize during screening. In the case of the bacteria (or other microorganism), the presence of too many or too few bacteria could, in some cases, make it more difficult to visualize the cells. Too many bacterial also, in some aspects, mean there would not be enough antibody secreted to coat or bind all of the bacteria.

The combined sample containing all components was warmed to 37° C. for 5 minutes. Approximately 125 μL of the melted agarose solution was added to the media containing the components, and approximately 100 μL of the encapsulation mixture was loaded on the μEncapsulator (Dolomite Microfluidics) chip, following the manufacturer's instructions to generate encapsulated particles in a non-aqueous environment. The encapsulated particles were collected and incubated on ice for 5-10 minutes to gel the agarose. The gelled encapsulated particles were transferred onto a 6-well dish containing 1 mL of 3M™ Novec™ 7500 as a non-aqueous gas permeable oil and incubated for 1 hour at 37° C., with agitation, to allow for antibody secretion by the B cells. The presence of the gas permeable oil allowed for physical separation of the droplets and ensured that the secreted antibody did not escape the non-aqueous environment, thereby resulting in a sufficiently high concentration of the antibody in the microdroplets for increased efficiency of the screening methods. The samples were then transferred into tubes, the emulsion was broken using Pico-Break (Dolomite Microfluidics), and washed with 2% fetal bovine serum (FBS) in PBS.

The particle samples were incubated with 20 μg/mL IgG (subclasses 1+2a+2b+3), Fcγ fragment specific secondary antibody conjugated to a green fluorophore (goat anti-mouse Fc-AF488; Jackson Immunoresearch Cat. No. 102646-750, diluted in 10% FBS/PBS) in the dark for 30 minutes on ice for visualization of produced antibodies. The samples were then washed in 2% FBS/PBS and stored on ice until ready to screen. Particles at a volume of approximately 300-500 μL (or up to 1 mL depending on the density) were placed onto a round coverglass bottom screening dish (Fisher Scientific, Cat. No. 14035-20). The coverglass bottom screening dish allowed for brighter high resolution imaging and visualization of fluorescent signal than a thicker imaging surface. Filtered PBS was added to a total volume not to exceed 2 mL and particles were allowed to settle to bottom of the dish. The particles were imaged and screened for antibody binding by visualization of a fluorescent signal using a fluorescent microscope. Particles were detected that co-encapsulated with fluorescent beads and/or fluorescent bacteria and antibody-producing cells. An antibody-producing cell from a particle that was positive for a signal was selected using a micromanipulator needle (Origio, Cat. No. C140819).

### Example 5: Use of A. baumannii Reporter Cells in Microparticle-Based Screen for Antibodies that Perturb the Gram-Negative Cell Envelope

An outer membrane (OM) stress transcriptional reporter A. baumannii cell was encapsulated with antibody-producing cells using the methods substantially described in Example 4, except that particles containing an antibody-producing cell that secreted an antibody that induced a phenotypic change in the bacteria were identified by induction of a fluorescent reporter molecule in the bacterial cell under the operable control of a regulatory region responsive to a modified phenotypic change involving cellular stress to the outer membrane.

To identify a regulatory region responsive to outer membrane stress, outer membrane stress was induced in A. baumannii by either depletion of BamA, an essential OM biogenesis factor, or by growth in the presence of polymyxin B nonapeptide (PMBN), which is an agent known to disrupt or permeabilize the outer membrane of Gram-negative bacteria. Changes in gene expression were assessed using RNA-Seq. Expression of approximately 790 genes was upregulated greater than 2-fold or more in response to one or both of the agents causing the modified phenotypic change. Expression of approximately 640 genes was downregulated greater than 2-fold or more in response to one or both of the agents causing the modified phenotypic change.

Exemplary reporter constructs were generated containing transcriptional regulatory regions upstream of exemplary genes identified as being upregulated greater than 10-fold. A DNA sequence upstream of the open reading frame (ORF) of each gene was coupled to a fluorescent reporter molecule. The fusion polypeptides were incorporated by assembly into an expression vector and introduced into A. baumannii to generate the reporter bacterial cell.

An antibody-producing cell pool was generated by immunizing Balb/C mice with A. baumannii Ab307-0294. Antibody-producing B cells were isolated from spleen and lymph node cells of the immunized Balb/C animals in a two-step cell purification process substantially as described in Example 4. First, spleen and lymph node cells were harvested and purified using the Pan B Cell Isolation Kit (Miltenyi Biotec, Cat. No. 130-095-813) to remove tissue debris and other material. Then, the cell preparation was further purified using EasySep™ CD138+ cell isolation kit (Stemcell Technologies) to obtain a B cell preparation.

Single B cells were co-encapsulated with reporter bacterial cells as described in Example 4, except in this exemplary experiment antigen-coated beads were not co-encapsulated. Also, because the bacterial cells express a reporter molecule the incubation with the secondary antibody to detect secreted antibodies was omitted.

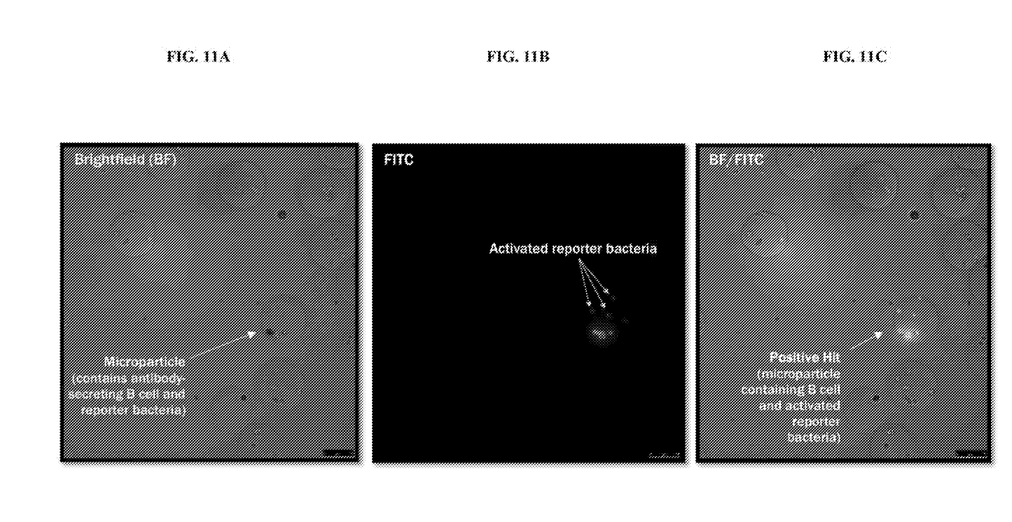

The particles were imaged and screened for antibody binding by visualization of a fluorescent signal using a fluorescent microscope. Particles were detected that co-encapsulated with fluorescent bacteria and antibody-producing cells. As shown in FIG. 11A-11C, particles containing fluorescent bacteria were observed, indicating the existence of an antibody-secreting B cell that secreted a molecule that bound to the cells in a manner to disrupt the outer membrane and/or induce an outer membrane stress. This B cell is then selected using a micromanipulator needle (Origio, Cat. No. C140819) for single-cell antibody cloning.

### Example 6: Purification and Preparation of Antigens for Immunization

This example describes methods to generate, purify and prepare exemplary outer membrane proteins as antigen for immunization, to obtain antibody-producing cells against the immunized protein.

A. Purification of Antigen: BamA

To generate and purify the barrel portion of A. baummannii BamA, E. coli BL21-DE3 cells were transformed with a plasmid encoding the barrel portion of A. baummannii BamA containing an N-terminal Avi-10His-TEV Tag (e.g., encoding a BamA variant set forth in SEQ ID NO:3). E. coli cells were cultured and expression of the protein was induced by the addition of isopropyl β-D-1-thiogalactopyranoside (IPTG). Cells were harvested and lysed by resuspending the cell pellet in lysis buffer (50 mM Tris pH 8, 150 mM NaCl, 20 μl DNAseI (25 μg/μl), 1 mM PMSF, 1 Roche Complete protease inhibitor per 50 ml) and lysozyme, and additionally homogenizing the cells using an LM-10 Microfluidizer® (Microfluidics, Westwood, Mass.) three times at 18,000 psi.

The homogenized samples were centrifuged and washed several times in wash buffer (50 mM Tris pH 8, 150 mM NaCl, 0.1 mM PMSF, 1 mM DTT, 0.5% Triton-X 100, Roche Complete protease inhibitor) and incubated in 8 M urea, 50 mM Tris pH 8, 150 mM NaCl, overnight at room temperature for solubilization. Samples were then centrifuged and passed through pre-packed 3 ml Co²⁺ column (ThermoScientific, Prod#89969), washed several times in UniA buffer (8 M urea, 50 mM Tris pH 7.4, 150 mM NaCl), followed by elution with UniB buffer (150 mM imidazole, 8 M urea, 50 mM Tris pH 7.4, 150 mM NaCl). Buffer exchange and sample concentration was performed, using 8 M urea, 50 mM Tris pH 7.4, 150 mM NaCl, 1 mM DTT and Amicon Ultra-15 device with a 10 kDa molecular weight cutoff (Millipore).

The prepared protein samples were refolded by adding 1 part of protein sample into 9 parts of 1.1× refolding buffer (55 mM Tris pH 8, 165 mM NaCl, 66 mM SDS, 1.65 MPD), mixing and incubating at room temperature for 3 days. The refolded protein samples were concentrated using an Amicon Ultra-15 device with a 10 kDa molecular weight cutoff (Millipore) and passed through Superdex 200 Increase 10/30 size exclusion column (GE healthcare, Pittsburgh, Pa.) on an AKTA Pure (GE healthcare) with running buffer (10 mM Hepes pH 8.0, 150 mM NaCl, 0.8% C8E4). The samples were verified on an SDS-PAGE gel, pooled and further concentrated.

B. Preparation for Immunization

The detergent or surfactant in the membrane protein preparation generated as described above was replaced with an amphipathic surfactant amphipol, to prepare for immunization. A solution of amphipol A8-35 was prepared in distilled water, at a concentration of a protein:amphipol ratio of 1:4 (e.g., 4 mg amphipol per mg of protein). Each antigen was dissolved in the amphipol solution at 2 mg/ml protein concentration, and incubated for 4 hours at 4° C. with gentle agitation. The protein/amphipol mixture was loaded onto a HiPrep 16/60 S-300 gel filtration column equilibrated in gel filtration buffer (20 mM Hepes pH 8.0, 150 mM NaCl), and the protein was eluted with the gel filtration buffer, to remove excess unbound amphipol or any remaining unbound detergent or surfactants remaining. The samples were verified on an SDS-PAGE gel, the protein/amphipol complex was pooled and further concentrated using an Amicon Ultra-15 device with a 10 kDa molecular weight cutoff (Millipore).

### Example 7: Illustrative Methods for Identifying Antibodies Binding to LptD

Hybridoma cells producing antibodies against A. baumannii LptD were generated from mice immunized with LptD. BALB/c mice were immunized with purified LptD/LptE complex. LptE is required for proper refolding of LptD. Antibody-producing cells were harvested from the spleens of mice showing a polyclonal serum response to purified LptD/LptE. Electrofusions were performed to generate hybridomas from discrete antibody-producing cells. Antibodies secreted by the hybridomas were collected as cell culture supernatants.

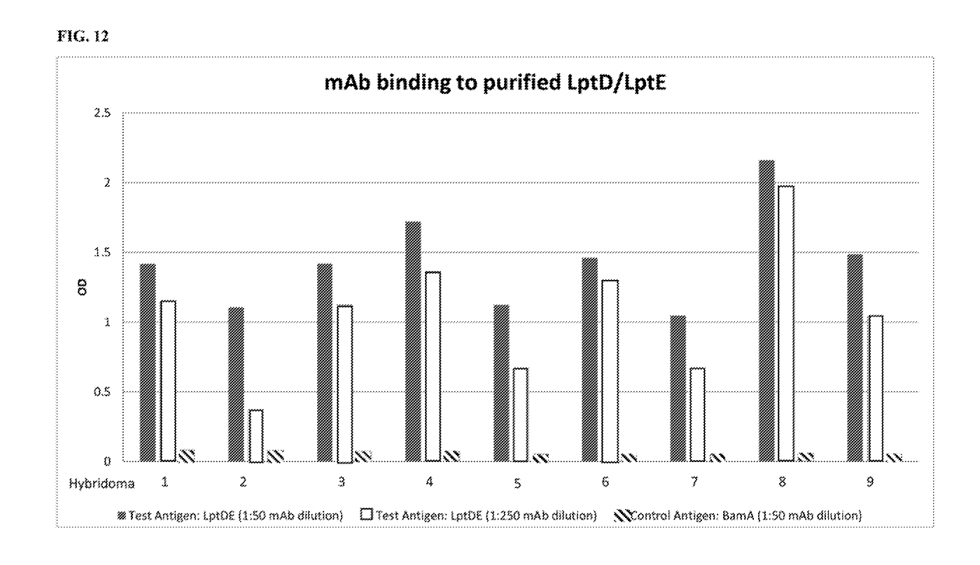

Binding of the antibodies obtained from the hybridoma cells were tested for binding to purified LptD/LptE and to a negative control antigen BamA by ELISA. LptD/LptE was tested at 1:50 and 1:250 mAb dilution, and the negative control BamA was tested at 1:50 mAb dilution. FIG. 12 shows the results from 9 independent hybridoma lines. The results show that the binding activity was titratable based on the amount of mAb added. The results show that all 9 monoclonal antibodies exhibit binding to LptD/LptE but not to the negative control antigen BamA, showing antigen-specific binding activity.

The hybridoma cells producing antibodies against LptD are encapsulated in agarose microdroplets with bacterial cells expressing LptD, and with beads conjugated with a variant of LptD, as described generally according to Example 4 above.

### Example 8: Humanized Antibodies to a Conserved Region of BamA

Transgenic mice genetically modified to produce humanized antibodies were immunized with A. baummannii BamA. Eight (8) humanized antibody-producing Trianni mice (TRIANNI, Inc) were immunized with 10 μg of purified preparation of barrel portion of BamA variant 1 set forth in SEQ ID NO:3, generated as described in Example 6 above. Cyclic di-nucleotide was used as an adjuvant, and the antigen preparation was injected in the footpad. Hyperimmunization was performed by footpad and subcutaneous injection.

Serum containing polyclonal antibody was obtained from each mouse at day 21 after immunization and at termination (terminal bleed). To test whether the Trianni donor mouse produces antibodies against a conserved region of BamA, the D21 sera and/or terminal bleed were tested for the presence of antibodies binding to a different variant of BamA. An A. baumanni test strain that conditionally expresses BamA variant 5 (set forth in SEQ ID NO:31) on the surface was generated. BamA variant 5 is a modified version of BamA variant 1, where the extracellular Loop 4, a loop that is highly variable between different isolates and variants of BamA, is replaced by the extracellular Loop 4 sequence of BamA variant 2. If an antibody obtained from immunization with BamA variant 1 also binds to BamA variant 5, the non-conserved hypervariable Loop 4 can be excluded from the epitopes recognized by the antibody.

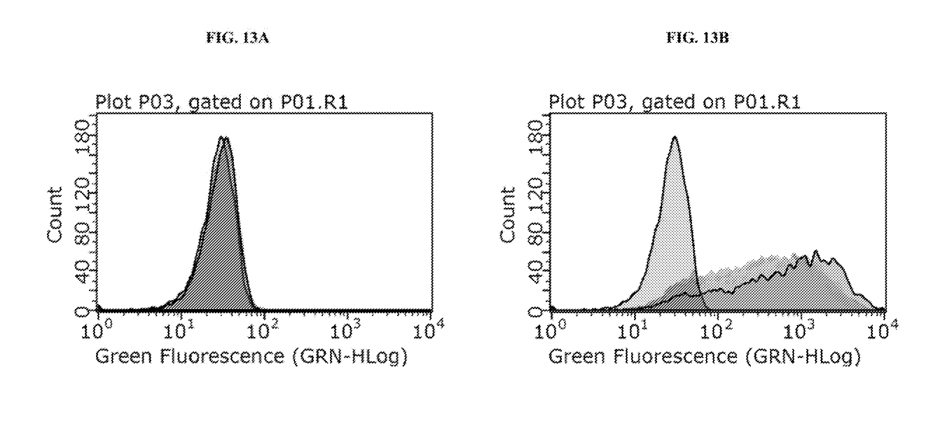

A 40× dilution of polyclonal sera from D21 and/or terminal bleed from the eight immunized mice was incubated with a A. baumannii control that did not express BamA (low) or the A. baumanni test strain expressing BamA variant 5 (high). Bound antibody was detected using a fluorescent labeled secondary antibody. FIG. 13A shows a histogram overlay of fluorescence signal after incubation with D21 and terminal bleed polyclonal sera for each of the eight mice binding to the control A. baumanni that did not express BamA. FIG. 13B shows a histogram overlay of fluorescence signal after incubation with terminal bleed polyclonal sera for each of the eight mice to A. baumanni test strain expressing BamA variant 5 (high). Table 1 shows the mean fluorescence intensity signal from the terminal bleed sera from each mouse for cell binding to BamA variant 5 divided by the background fluorescence signal from the control. As shown, the terminal bleed response showed an enriched binding signal to the non-loop 4 region of BamA, indicating binding to a conserved region of the extracellular portion.

TABLE 1
Cell Binding Response to
BamA Variant 5

| | Differential Signal |
|---|---|
| Mouse | (High/Low) |
| No. | Terminal Bleed |
| 28 | 10 |
| 29 | 14 |
| 58 | 13 |
| 60 | 12 |
| 68 | 6 |
| 95 | 8 |
| 105 | 11 |
| 109 | 19 |

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| # | SEQUENCE | ANNOTATION |
|---|---|---|
| 1 | EEQHSGTTTLAVGYSQSGGITFQAGLSQTNFMGTGNRVAIDLSRSE | Acinetobacter baumannii |
| | TQDYYNLSVTDPYFTIDGVSRGYNVYYRKTKLNDDYNVNNYVTDSF | BamA variant 1 (N-terminal |
| | GGSLSFGYPIDENQSLSASVGVDNTKVTTGAFVSTYVRDYLLANGG | deletion) |
| | KTTSTNTYCLVDLVQDPQTGLYKCPEGQTSQPYGNAFEGEFFTYNL | |
| | NLGWSYNTLNRPIFPTSGMSHRVGLEIGLPGSDVDYQKVTYDTQAF | |
| | FPIGSTGFVLRGYGKLGYGNDLPFYKNFYAGGYGSVRGYDNSTLGP | |
| | KYASVNLQEEKKNDSSPEEVGGNALVQFGTELVLPMPFKGDWTRQV | |
| | RPVLFAEGGQVFDTKCDVRSYSMIMNGQQISDAKKYCEDNYGFDLG | |
| | NLRYSVGVGVTWITMIGPLSLSYAFPLNDKPGDETKEIQFEIGRTF | |
| 2 | DDFVVRDIRVNGLVRLTPANVYTMLPINSGDRVNEPMIAEAIRTLY | A. baumannii BamA variant |
| | ATGLFDDIKASKENDTLVFNVIERPIISKLEFKGNKLIPKEALEQG | 2 full length (ATCC 19606) |
| | LKKMGIAEGEVFKKSALQTIETELEQQYTQQGRYDADVTVDTVARP | |
| | NNRVELKINFNEGTPAKVFDINVIGNTVFKDSEIKQAFAVKESGWA | |
| | SVVTRNDRYAREKMAASLEALRAMYLNKGYINFNINNSQLNISEDK | |
| | KHIFIEVAVDEGSQFKFGQTKFLGDALYKPEELQALKIYKDGDTYS | |
| | QEKVNAVKQLLLRKYGNAGYYFADVNIVPQINNETGVVDLNYYVNP | |
| | GQQVTVRRINFTGNSKTSDEVLRREMRQMEGALASNEKIDLSKVRL | |
| | ERTGFFKTVDIKPARIPNSPDQVDLNVNVEEQHSGTTTLAVGYSQS | |
| | GGITFQAGLSQTNFMGTGNRVAIDLSRSETQDYYNLSVTDPYFTID | |
| | GVSRGYNVYYRKTKLNDDYNVNNYVTDSFGGSLSFGYPIDENQSLS | |
| | ASVGVDNTKVTTGPYVSTYVRDYLLANGGKATSKGTYCPTDANGDS | |
| | QYDTEKGECKVPEETYDNAFEGEFFTYNLNLGWSYNTLNRPIFPTS | |
| | GMSHRVGLEIGLPGSDVDYQKVTYDTQAFFPIGSTGFVLRGYGKLG | |
| | YGNDLPFYKNFYAGGYGSVRGYDNSTLGPKYPSVNLQETKQNDSSP | |
| | EEVGGNALVQFGTELVLPMPFKGDWTRQVRPVLFAEGGQVFDTKCN | |
| | IDNSVYGNKGMKINGQTITDVRKYCEDNYGFDLGNLRYSVGVGVTW | |
| | ITMIGPLSLSYAFPLNDKPGDETKEIQFEIGRTF | |
| 3 | MSGLNDIFEAQKIEWHEGAHHHHHHHHHHDYDIPTSENLYFQGASE | AviTag-10xHis-TEV-A. |
| | EQHSGTTTLAVGYSQSGGITFQAGLSQTNFMGTGNRVAIDLSRSET | baumannii BamA variant 1 |
| | QDYYNLSVTDPYFTIDGVSRGYNVYYRKTKLNDDYNVNNYVTDSFG | (N-terminal deletion) |
| | GSLSFGYPIDENQSLSASVGVDNTKVTTGAFVSTYVRDYLLANGGK | |
| | TTSTNTYCLVDLVQDPQTGLYKCPEGQTSQPYGNAFEGEFFTYNLN | |
| | LGWSYNTLNRPIFPTSGMSHRVGLEIGLPGSDVDYQKVTYDTQAFF | |
| | PIGSTGFVLRGYGKLGYGNDLPFYKNFYAGGYGSVRGYDNSTLGPK | |
| | YASVNLQEEKKNDSSPEEVGGNALVQFGTELVLPMPFKGDWTRQVR | |
| | PVLFAEGGQVFDTKCDVRSYSMIMNGQQISDAKKYCEDNYGFDLGN | |
| | LRYSVGVGVTWITMIGPLSLSYAFPLNDKPGDETKEIQFEIGRTF | |
| 4 | MGSSHHHHHHSSGLVPRGSHMASADDFVVRDIRVNGLVRLTPANVY | 6xHis-A. baumannii BamA |
| | TMLPINSGDRVNEPMIAEAIRTLYATGLFDDIKASKENDTLVFNVI | variant 2 |
| | ERPIISKLEFKGNKLIPKEALEQGLKKMGIAEGEVFKKSALQTIET | |
| | ELEQQYTQQGRYDADVTVDTVARPNNRVELKINFNEGTPAKVFDIN | |
| | VIGNTVFKDSEIKQAFAVKESGWASVVTRNDRYAREKMAASLEALR | |
| | AMYLNKGYINFNINNSQLNISEDKKHIFIEVAVDEGSQFKFGQTKF | |
| | LGDALYKPEELQALKIYKDGDTYSQEKVNAVKQLLLRKYGNAGYYF | |
| | ADVNIVPQINNETGVVDLNYYVNPGQQVTVRRINFTGNSKTSDEVL | |
| | RREMRQMEGALASNEKIDLSKVRLERTGFFKTVDIKPARIPNSPDQ | |
| | VDLNVNVEEQHSGTTTLAVGYSQSGGITFQAGLSQTNFMGTGNRVA | |
| | IDLSRSETQDYYNLSVTDPYFTIDGVSRGYNVYYRKTKLNDDYNVN | |
| | NYVTDSFGGSLSFGYPIDENQSLSASVGVDNTKVTTGPYVSTYVRD | |
| | YLLANGGKATSKGTYCPTDANGDSQYDTEKGECKVPEETYDNAFEG | |
| | EFFTYNLNLGWSYNTLNRPIFPTSGMSHRVGLEIGLPGSDVDYQKV | |
| | TYDTQAFFPIGSTGFVLRGYGKLGYGNDLPFYKNFYAGGYGSVRGY | |
| | DNSTLGPKYPSVNLQETKQNDSSPEEVGGNALVQFGTELVLPMPFK | |
| | GDWTRQVRPVLFAEGGQVFDTKCNIDNSVYGNKGMKINGQTITDVR | |
| | KYCEDNYGFDLGNLRYSVGVGVTWITMIGPLSLSYAFPLNDKPGDE | |
| | TKEIQFEIGRTF | |
| 5 | EEQHSGTTTLAVGYSQSGGITFQAGLSQTNFMGTGNRVAIDLSRSE | BamA variant 3 (N-terminal |
| | TQDYYNLSVTDPYFTIDGVSRGYNVYYRKTKLNDDYNVNNYVTDSF | deletion) |
| | GGSLSFGYPIDENQSLSASVGVDNTKVTTGPYVSTYVRDYLLANGG | |
| | KATSKGTYCPTDANGDSQYDTEKGECKVPEETYDNAFEGEFFTYNL | |
| | NLGWSYNTLNRPIFPTSGMSHRVGLEIGLPGSDVDYQKVTYDTQAF | |
| | FPIGSTGFVLRGYGKLGYGNDLPFYKNFYAGGYGSVRGYDNSTLGP | |
| | KYPSVNLQETKQNDSSPEEVGGNALVQFGTELVLPMPFKGDWTRQV | |
| | RPVLFAEGGQVFDTKCNIDNSVYGNKGMKINGQTITDVRKYCEDNY | |
| | GFDLGNLRYSVGVGVTWITMIGPLSLSYAFPLNDKPGDETKEIQFE | |
| | IGRTF | |
| 6 | EEQHSGTTTLAVGYSQSGGITFQAGLSQTNFMGTGNRVAIDLSRSE | BamA variant 4(N-terminal |
| | TQDYYNLSVTDPYFTIDGVSRGYNVYYRKTKLNDDYNVNNYVTDSF | deletion) |
| | GGSLSFGYPIDENQSLSASVGVDNTKVTTGPYVSTYVRDYLLANGG | |
| | KATGKSSWCPTGKNEVDPKTQQPIPNTCEGGFEPYESAFEGEFFTY | |
| | NLNLGWSYNTLNRPIFPTSGMSHRVGLEIGLPGSDVDYQKVTYDTQ | |
| | AFFPIGSTGFVIRGYGKLGYGNDLPFYKNFYAGGYGSVRGYDNSTL | |
| | GPKYASVNLQETKQNDGSPEEVGGNALVQFGTELVLPMPFKGDWTR | |
| | QVRPVLFAEGGQVFDTKCNIDNTVYGDKGMKINGQTITDVRKYCED | |
| | NYGFDLGNLRYSVGVGVTWITMIGPLSLSYAFPLNDKPGDETKEIQ | |
| | FEIGRTF | |
| 7 | MSGLNDIFEAQKIEWHEGAHHHHHHHHHHDYDIPTSENLYFQGASE | AviTag-10xHis-TEV-A. |
| | EQHSGTTTLAVGYSQSGGITFQAGLSQTNFMGTGNRVAIDLSRSET | baumannii BamA variant 4 |
| | QDYYNLSVTDPYFTIDGVSRGYNVYYRKTKLNDDYNVNNYVTDSFG | (N-terminal deletion) |
| | GSLSFGYPIDENQSLSASVGVDNTKVTTGPYVSTYVRDYLLANGGK | |
| | ATSKGTYCPTDANGDSQYDTEKGECKVPEETYDNAFEGEFFTYNLN | |
| | LGWSYNTLNRPIFPTSGMSHRVGLEIGLPGSDVDYQKVTYDTQAFF | |
| | PIGSTGFVLRGYGKLGYGNDLPFYKNFYAGGYGSVRGYDNSTLGPK | |
| | YPSVNLQETKQNDSSPEEVGGNALVQFGTELVLPMPFKGDWTRQVR | |
| | PVLFAEGGQVFDTKCNIDNSVYGNKGMKINGQTITDVRKYCEDNYG | |
| | FDLGNLRYSVGVGVTWITMIGPLSLSYAFPLNDKPGDETKEIQFEI | |
| | GRTF | |
| 8 | MSGLNDIFEAQKIEWHEGAHHHHHHHHHHDYDIPTSENLYFQGASE | AviTag-10xHis-TEV-A. |
| | EQHSGTTTLAVGYSQSGGITFQAGLSQTNFMGTGNRVAIDLSRSET | baumannii BamA variant 4 |
| | QDYYNLSVTDPYFTIDGVSRGYNVYYRKTKLNDDYNVNNYVTDSFG | (N-terminal deletion) |
| | GSLSFGYPIDENQSLSASVGVDNTKVTTGPYVSTYVRDYLLANGGK | |
| | ATGKSSWCPTGKNEVDPKTQQPIPNTCEGGFEPYESAFEGEFFTYN | |
| | LNLGWSYNTLNRPIFPTSGMSHRVGLEIGLPGSDVDYQKVTYDTQA | |
| | FFPIGSTGFVIRGYGKLGYGNDLPFYKNFYAGGYGSVRGYDNSTLG | |
| | PKYASVNLQETKQNDGSPEEVGGNALVQFGTELVLPMPFKGDWTRQ | |
| | VRPVLFAEGGQVFDTKCNIDNTVYGDKGMKINGQTITDVRKYCEDN | |
| | YGFDLGNLRYSVGVGVTWITMIGPLSLSYAFPLNDKPGDETKEIQF | |
| | EIGRTF | |
| 9 | MSGLNDIFEAQKIEWHEGAHHHHHHHHHHDYDIPTSENLYFQGAS | AviTag-10xHis-TEV |
| 10 | MGSSHHHHHHSSGLVPRGSHMASA | 6xHis |
| 11 | MRHTHFLMPLALVSAMAAVQQAYAADDFVVRDIRVNGLVRLTPANV | A. baumannii ATCC 19606 |
| | YTMLPINSGDRVNEPMIAEAIRTLYATGLFDDIKASKENDTLVFNV | full length including signal |
| | IERPIISKLEFKGNKLIPKEALEQGLKKMGIAEGEVFKKSALQTIE | sequence |
| | TELEQQYTQQGRYDADVTVDTVARPNNRVELKINFNEGTPAKVFDI | |
| | NVIGNTVFKDSEIKQAFAVKESGWASVVTRNDRYAREKMAASLEAL | |
| | RAMYLNKGYINFNINNSQLNISEDKKHIFIEVAVDEGSQFKFGQTK | |
| | FLGDALYKPEELQALKIYKDGDTYSQEKVNAVKQLLLRKYGNAGYY | |
| | FADVNIVPQINNETGVVDLNYYVNPGQQVTVRRINFTGNSKTSDEV | |
| | LRREMRQMEGALASNEKIDLSKVRLERTGFFKTVDIKPARIPNSPD | |
| | QVDLNVNVEEQHSGTTTLAVGYSQSGGITFQAGLSQTNFMGTGNRV | |
| | AIDLSRSETQDYYNLSVTDPYFTIDGVSRGYNVYYRKTKLNDDYNV | |
| | NNYVTDSFGGSLSFGYPIDENQSLSASVGVDNTKVTTGPYVSTYVR | |
| | DYLLANGGKATSKGTYCPTDANGDSQYDTEKGECKVPEETYDNAFE | |
| | GEFFTYNLNLGWSYNTLNRPIFPTSGMSHRVGLEIGLPGSDVDYQK | |
| | VTYDTQAFFPIGSTGFVLRGYGKLGYGNDLPFYKNFYAGGYGSVRG | |
| | YDNSTLGPKYPSVNLQETKQNDSSPEEVGGNALVQFGTELVLPMPF | |
| | KGDWTRQVRPVLFAEGGQVFDTKCNIDNSVYGNKGMKINGQTITDV | |
| | RKYCEDNYGFDLGNLRYSVGVGVTWITMIGPLSLSYAFPLNDKPGD | |
| | ETKEIQFEIGRTF | |
| 12 | EEQHSGTTTLAVGYSQ | A. baumannii ATCC 19606 |
| | | BamA residues 423-438 |
| 13 | GGITFQAGLSQTNFMGTGNRV | A. baumannii ATCC 19606 |
| | | BamA residues 440-460 |
| 14 | IDLSRSETQDYYNLSVTDPYFTIDGVSRGYNVYYRKTKLND | A. baumannii ATCC 19606 |
| | | BamA residues 462-502 |
| 15 | YNVNNYVTDSFGGSLSFGYPIDENQSLSAS | A. baumannii ATCC 19606 |
| | | BamA residues 504-533 |
| 16 | DNTKVTTG | A. baumannii ATCC 19606 |
| | | BamA residues 537-544 |
| 17 | VSTYVRDYL | A. baumannii ATCC 19606 |
| | | BamA residues 547-555 |
| 18 | ANGGK | A. baumannii ATCC 19606 |
| | | BamA residues 557-561 |
| 19 | GEFFTY | A. baumannii ATCC 19606 |
| | | BamA residues 599-604 |
| 20 | LNLGWSYNTLNRPIFPTSGMSHRVGLEIGLPGSDVDYQK | A. baumannii ATCC 19606 |
| | | BamA residues 606-644 |
| 21 | TYDTQAF | A. baumannii ATCC 19606 |
| | | BamA residues 646-652 |
| 22 | GFVLRGYGKLGYGNDLPFYKNFYAGGYGSVRGYDNSTLGPKY | A. baumannii ATCC 19606 |
| | | BamA residues 659-700 |
| 23 | SVNLQE | A. baumannii ATCC 19606 |
| | | BamA residues 702-707 |
| 24 | VGGNAL | A. baumannii ATCC 19606 |
| | | BamA residues 718-723 |
| 25 | PFKGDWTRQVRPV | A. baumannii ATCC 19606 |
| | | BamA residues 735-747 |
| 26 | FAEGGQVFDTKC | A. baumannii ATCC 19606 |
| | | BamA residues 749-760 |
| 27 | KYCEDNYGFDL | A. baumannii ATCC 19606 |
| | | BamA residues 784-794 |
| 28 | RYSVGVG | A. baumannii ATCC 19606 |
| | | BamA residues 798-804 |
| 29 | TWITMIGPLS | A. baumannii ATCC 19606 |
| | | BamA residues 806-815 |
| 30 | SYAFPLNDKPGDETKEIQFEIGRTF | A. baumannii ATCC 19606 |
| | | BamA residues 817-841 |
| 31 | EEQHSGTTTLAVGYSQSGGITFQAGLSQTNFMGTGNRVAIDLSRSE | A. baumannii BamA variant |
| | TQDYYNLSVTDPYFTIDGVSRGYNVYYRKTKLNDDYNVNNYVTDSF | 5 (N-terminal deletion) |
| | GGSLSFGYPIDENQSLSASVGVDNTKVTTGPYVSTYVRDYLLANGG | |
| | KATSKGTYCPTDANGDSQYDTEKGECKVPEETYDNAFEGEFFTYNL | |
| | NLGWSYNTLNRPIFPTSGMSHRVGLEIGLPGSDVDYQKVTYDTQAF | |
| | FPIGSTGFVLRGYGKLGYGNDLPFYKNFYAGGYGSVRGYDNSTLGP | |
| | KYASVNLQEEKKNDSSPEEVGGNALVQFGTELVLPMPFKGDWTRQV | |
| | RPVLFAEGGQVFDTKCDVRSYSMIMNGQQISDAKKYCEDNYGFDLG | |
| | NLRYSVGVGVTWITMIGPLSLSYAFPLNDKPGDETKEIQFEIGRTF | |

What is claimed:

1. A method for identifying an antibody that binds a target microorganism, comprising:
(a) obtaining a plurality of candidate antibody-producing cells;
(b) encapsulating the plurality of candidate antibody-producing cells in gel microdroplets with a target microorganism; and
(c) determining whether the antibody-producing cell(s) within the gel microdroplet produce an antibody that binds the target microorganism, thereby identifying an antibody that specifically binds to the target microorganism.

2. The method of claim 1, wherein:
step (b) further comprises encapsulating, in the microdroplets, an epitope-comprising fragment of the target microorganism or a variant thereof; and
step (c) comprises determining whether the antibody identified as binding the target microorganism also binds the epitope-comprising fragment thereof within the same gel microdroplet.

3. A method for identifying an antibody that binds a target microorganism, comprising:
(a) obtaining a plurality of candidate antibody-producing cells;
(b) encapsulating the plurality of candidate antibody-producing cells in gel microdroplets with a target microorganism and with an epitope-comprising fragment of the target microorganism or a variant thereof; and
(c) determining whether the antibody-producing cell(s) within the gel microdroplet produce an antibody that binds the target microorganism and/or epitope-comprising fragment thereof present in the same gel microdroplet, thereby identifying an antibody that specifically binds to the target microorganism or epitope-comprising fragment thereof.

4. The method of any of claims 1-3, wherein the epitope-comprising fragment is bound to a solid support.

5. The method of claim 4, wherein the solid support is a bead.

6. The method of any of claims 1-5, wherein the target microorganism is a bacterium, a fungus, a parasite or a virus.

7. The method of claim 6, wherein the target microorganism is a bacterium or a fungus.

8. The method of claim 6 or claim 7, wherein the microorganism is a multi-drug resistant microorganism.

9. The method of any of claims 6-8, wherein the microorganism is a bacterium that is a Gram-negative bacterium.

10. The method of claim 9, wherein the Gram-negative bacterium is a proteobacterium.

11. The method of any of claims 6-10, wherein the microorganism is a bacterium selected from among a species of Acinetobacter, Bdellovibrio, Burkholderia, Chlamydia, Enterobacter, Escherichia, Francisella, Haemophilus, Helicobacter, Klebsiella, Legionella, Moraxella, Neisseria, Pantoea, Pseudomonas, Salmonella, Shigella, Stenotrophomonas, Vibrio and Yersinia.

12. The method of any of claims 6-11, wherein the microorganism is selected from among Acinetobacter apis, Acinetobacter baumannii, Acinetobacter baylyi, Acinetobacter beijerinckii, Acinetobacter bereziniae, Acinetobacter bohemicus, Acinetobacter boissieri, Acinetobacter bouvetii, Acinetobacter brisouii, Acinetobacter calcoaceticus, Acinetobacter gandensis, Acinetobacter gerneri, Acinetobacter guangdongensis, Acinetobacter guillouiae, Acinetobacter gyllenbergii, Acinetobacter haemolyticus, Acinetobacter harbinensis, Acinetobacter indicus, Acinetobacter johnsonii, Acinetobacter junii, Acinetobacter kookii, Acinetobacter lwoffii, Acinetobacter nectaris, Acinetobacter nosocomialis, Acinetobacter pakistanensis, Acinetobacter parvus, Acinetobacter pitii, Acinetobacter pittii, Acinetobacter puyangensis, Acinetobacter qingfengensis, Acinetobacter radioresistans, Acinetobacter radioresistens, Acinetobacter rudis, Acinetobacter schindleri, Acinetobacter seifertii, Acinetobacter soli, Acinetobacter tandoii, Acinetobacter tjernbergiae, Acinetobacter towneri, Acinetobacter ursingii, Acinetobacter variabilis, Acinetobacter venetianus, Escherichia coli, Haemophilus influenzae, Klebsiella pneumoniae, Pseudomonas aeruginosa, Salmonella typhimurium, Shigella boydii, Shigella dysenteriae, Shigella flexneri, Shigella sonnei, Vibrio cholera and Yersinia pestis.

13. The method of claim 12, wherein the microorganism is Acinetobacter baumannii.

14. The method of any of claims 6-8, wherein the microorganism is a bacterium that is a Gram-positive bacterium.

15. The method of claim 14, wherein the microorganism is selected from among a species of Staphylococcus and Streptococcus.

16. The method of any of claims 6-8, wherein the microorganism is a fungus that is an Aspergillus species or a Candida species.

17. The method of claim 6 or claim 8, wherein the microorganism is a parasite that is a Coccidia or a Plasmodium species.

18. The method of any of claims 1-17, wherein the plurality of candidate antibody-producing cells are obtained from a donor that has been exposed to the target microorganism or an epitope-comprising fragment of the target microorganism or a variant thereof.

19. The method of any of claims 1-18, wherein the plurality of candidate antibody-producing cells is obtained by a method comprising:
(i) expanding antibody-producing cells obtained from a donor that has been exposed to the target microorganism or an epitope-comprising fragment of the target microorganism or a variant thereof by introducing a cell composition comprising the antibody-producing cells into an immunocompromised animal; and
(ii) recovering the expanded antibody-producing cells, thereby obtaining the plurality of candidate antibody-producing cells.

20. The method of claim 19, wherein the cell composition comprising the antibody-producing cells comprises cells obtained from the spleen and/or lymph node of the donor.

21. The method of claim 19 or claim 20, wherein the cell composition comprises T cells.

22. The method of any of claims 19-21, wherein the cell composition comprises peripheral blood mononuclear cells (PBMCs) comprising the antibody-producing cells.

23. The method of any of claims 19-22, wherein the immunocompromised animal is a SCID mouse.

24. The method of any of claims 19-23, wherein the cell composition comprising the antibody-producing cells is introduced into the immunocompromised animal intravenously or by transplant into the immunocompromised animal's spleen.

25. The method of any of claims 19-24, wherein:
the antibody-producing cells are from a donor exposed to a first variant of the target microorganism or epitope-comprising fragment thereof, and
prior to introducing the cell composition comprising the antibody-producing cells into the immunocompromised animal, the method comprises mixing or incubating the antibody-producing cells with a second variant of the target microorganism or epitope-comprising fragment thereof, wherein the introduced cell composition comprises the antibody-producing cells complexed with the second variant of the target microorganism or epitope-comprising fragment thereof.

26. The method of any of claims 1-25, wherein the epitope-comprising fragment comprises an essential protein or fragment of an essential protein of the target microorganism.

27. The method of any of claims 1-26, wherein the epitope-comprising fragment comprises a bacterial outer membrane (OM) protein, a membrane protein, an envelope proteins, a cell wall protein, a cell wall component, a surface lipid, a glycolipid, a lipopolysaccharide, a glycoprotein, a surface polysaccharide, a capsule, a surface appendage, a flagellum, a pilus, a monomolecular surface layer, or an S-layer or a fragment thereof derived from the target microorganism.

28. The method of any of claims 1-27, wherein the epitope-comprising fragment comprises a lipid from the surface of the target microorganism.

29. The method of claim 28, wherein the epitope-comprising fragment comprises a lipopolysaccharide (LPS) or a lipoprotein.

30. The method of any of claims 1-27, wherein the epitope-comprising fragment comprises an outer membrane (OM) protein.

31. The method of claim 30, wherein the OM protein is selected from among BamA, LptD, AdeC, AdeK, BtuB, FadL, FecA, FepA, FhaC, FhuA, LamB, MepC, MexA, NalP, NmpC, NspA, NupA, Omp117, Omp121, Omp200, Omp71, OmpA, OmpC, OmpF, OmpG, OmpT, OmpW, OpcA, OprA, OprB, OprF, OprJ, OprM, OprN, OstA, PagL, PagP, PhoE, PldA, PorA, PorB, PorD, PorP, SmeC, SmeF, SrpC, SucY, TolC, TtgC and TtgF.

32. The method of claim 31, wherein the OM protein is BamA or LptD.

33. The method of any of claims 25-27 and 30-32, wherein the epitope-comprising fragment is prepared by solubilization of the OM protein or a fragment thereof.

34. The method of claim 33, wherein solubilization is carried out by addition of one or more detergent or surfactant.

35. The method of claim 33 or claim 34, further comprising refolding of the epitope-comprising fragment prior to mixing or incubating with the antibody-producing cells.

36. The method of claim 35, wherein the refolding is carried out in the presence of one or more detergent or surfactant.

37. The method of any of claims 34-36, wherein the detergent or surfactant is selected from among lauryldimethylamine oxide (LDAO), 2-methyl-2,4-pentanediol (MPD), an amphipol, amphipol A8-35, C8E4, Triton X-100, octylglucoside, DM (n-Decyl-β-D-maltopyranoside), DDM (n-Dodecyl-β-D-maltopyranoside, 3-[(3-Cholamidopropyl)dimethylammonio]-1-propanesulfonate (CHAPS) and 3-[(3-cholamidopropyl)dimethylammonio]-2-hydroxy-1-propanesulfonate (CHAPSO).

38. The method of any of claims 34-37, further comprising replacing some or all of the detergent and/or surfactant in the preparation with an amphipathic polymer or a surfactant.

39. The method of any of claims 34-38, wherein prior to mixing or incubating with the antibody-producing cells, excess detergent or surfactant is removed or reduced from the preparation of the epitope-comprising fragment to a level or amount that is not toxic to and/or does not induce lysis of the antibody-producing cells.

40. The method of any of claims 25-39, wherein the first and second variant each independently comprises an epitope-comprising fragment of the target microorganism.

41. The method of any of claims 25-40, wherein the first and the second variant shares at least one conserved region or domain.

42. The method of claim 41, wherein the first and the second variant each comprise at least one region or domain that differs from each other.

43. The method of any of claims 25-42, wherein the first and second variant comprises an OM protein or fragment thereof derived from two different clinical isolates of the same microorganism.

44. The method of any of claims 25-43, wherein the first variant and/or second variant is a full-length OM protein and the other of the first and/or second variant is a fragment of the OM protein comprising deletion of an immunodominant epitope or loop of the OM protein.

45. The method of any of claims 41-44, wherein the identified antibody binds to the at least one conserved region or domain of the target microorganism.

46. The method of any of claims 18-45, wherein the donor has been immunized or infected with the target microorganism or an epitope-comprising fragment of the target microorganism or a variant thereof.

47. The method of any of claims 18-46, wherein the donor is an immunized animal or an infected animal.

48. The method of any of claims 18-47, wherein the donor is a mammal or a bird.

49. The method of any of claims 18-48, wherein the donor is a human, a mouse or a chicken.

50. The method of any of claims 18-49, wherein the donor is a human donor who was infected by the microorganism.

51. The method of any of claims 18-50, wherein the donor is a genetically modified non-human animal that produces partially human or fully human antibodies.

52. The method of any of claims 1-51, wherein the antibody-producing cells comprise peripheral blood mononuclear cells (PBMCs), B cells, plasmablasts or plasma cells.

53. The method of any of claims 1-52, wherein the antibody-producing cells comprise B cells, plasmablasts or plasma cells.

54. The method of any of claims 18-53, wherein the plurality of candidate antibody-producing cells are selected from the donor by a positive or negative selection to isolate or enrich for B cells.

55. The method of claim 54, wherein the B cell is a plasmablast or a plasma cell.

56. The method of claim 55, wherein the selection is a positive selection based on expression of a cell surface marker selected from among one or more of: CD2, CD3, CD4, CD14, CD15, CD16, CD34, CD56, CD61, CD138, CD235a (Glycophorin A) and FceRIa.

57. The method of any of claims 52-56, wherein the antibody-producing cells comprise CD138+ cells.

58. The method of any of claims 52-57, wherein at least or at least about 50%, 60%, 70%, 80%, 85%, 90%, 95%, or more of the cells are plasma cells or plasmablasts and/or are CD138+ cells.

59. The method of any of claims 1-58, wherein the antibody is an antibody or an antigen-binding fragment thereof.

60. The method of any of claims 1-59, wherein the gel microdroplet is generated by a microfluidics-based method.

61. The method of any of claims 1-60, wherein the gel microdroplet comprises material selected from among agarose, carrageenan, alginate, alginate-polylysine, collagen, cellulose, methylcellulose, gelatin, chitosan, extracellular matrix, dextran, starch, inulin, heparin, hyaluronan, fibrin, polyvinyl alcohol, poly(N-vinyl-2-pyrrolidone), polyethylene glycol, poly(hydroxyethyl methacrylate), acrylate polymers and sodium polyacrylate, polydimethyl siloxane, cis-polyisoprene, Puramatrix™, poly-divenylbenzene, polyurethane, or polyacrylamide or combinations thereof.

62. The method of claim 61, wherein the gel microdroplet comprises agarose.

63. The method of claim 62, wherein the agarose is low gelling temperature agarose.

64. The method of claim 62 or claim 63, wherein the agarose has a gelling temperature of lower than about 35° C., about 30° C., about 25° C., about 20° C., about 15° C., about 10° C. or about 5° C.

65. The method of claim 62 or claim 63, wherein the agarose has a gelling temperature of between about 5° C. and about 30° C., about 5° C. and about 20° C., about 5° C. and about 15° C., about 8° C. and about 17° C. or about 5° C. and about 10° C.

66. The method of any of claims 1-65, wherein step (b) further comprises incubating the gel microdroplets at a temperature of between about 0° C. and about 5° C. for about 1 minute to about 10 minutes subsequent to encapsulation.

67. The method of any of claims 5-66, wherein the bead has an average diameter of between about 100 nm and about 100 μm, or between about 3 μm and about 5 μm.

68. The method of any of claims 1-67, wherein the average ratio of candidate antibody-producing cell per gel microdroplet is less than or less than about 1.

69. The method of any of claims 1-68, wherein the average ratio of candidate antibody-producing cell per gel microdroplet is between about 0.05 and about 1.0, about 0.05 and about 0.5, about 0.05 and about 0.25, about 0.05 and about 0.1, about 0.1 and about 1.0, about 0.1 and about 0.5, about 0.1 and about 0.25, about 0.25 and about 1.0, about 0.25 and about 0.5 or 0.5 and about 1.0, each inclusive.

70. The method of claim 69, wherein the average ratio of candidate antibody-producing cells per microdroplet is or is about 0.1.

71. The method of any of claims 1-70, wherein the average ratio of the microorganism per gel microdroplet is between about 50 and about 150 or about 50 and about 100.

72. The method of any of claims 5-71, wherein the average ratio of the bead per gel microdroplet is between about 2 and about 10 or about 3 and about 5.

73. The method of any of claims 5-72, wherein the average ratio of the candidate cell to microorganism to bead is about 0.1:100:10.

74. The method of any of claims 1-73, wherein the gel microdroplets comprise growth media and are surrounded by a non-aqueous environment.

75. The method of claim 74, wherein the non-aqueous environment comprises an oil.

76. The method of claim 75, wherein the oil is gas permeable.

77. The method of any of claims 1-76, further comprising incubating the gel microdroplets at a temperature of at or about 37° C. prior to step (c).

78. The method of claim 77, wherein the gel microdroplets are incubated in growth media.

79. The method of any of claims 1-78, wherein prior to step (c), introducing into the gel microdroplets a reagent that binds to antibodies, said reagent comprising a detectable moiety.

80. The method of claim 79, wherein the reagent comprises a secondary antibody specific for antibodies produced by the encapsulated antibody-producing cells.

81. The method of claim 79 or claim 80, wherein determining whether the antibody-producing cell(s) within the gel microdroplet produce an antibody that binds the target microorganism and/or epitope-comprising fragment thereof present in the same gel microdroplet comprises detecting the presence of a complex comprising: (i) the target microorganism or epitope-comprising fragment thereof; (ii) the antibody produced by the antibody-producing cell; and (iii) the reagent comprising the detectable moiety bound, wherein the presence of the complex indicates that the antibody specifically binds the target microorganism or epitope-comprising fragment thereof.

82. The method of any of claims 1-78, wherein determining whether the antibody-producing cell(s) within the gel microdroplet produce an antibody that binds the target microorganism and/or epitope-comprising fragment thereof present in the same gel microdroplet comprises determining whether the presence of the antibody modifies a phenotypic characteristic of the target microorganism in the same gel microdroplet, wherein the presence of the modified phenotypic characteristic indicates that the antibody specifically binds the target microorganism or epitope-comprising fragment thereof.

83. The method of claim 82, wherein the modified phenotypic characteristic is selected from among cell growth, cell death, changes in in behavior, binding, transcription, translation, expression, protein transport, cellular or membrane architecture, adhesion, motility, cellular stress, cell division and/or cell viability.

84. The method of claim 82 or claim 83, wherein determining whether the antibody-producing cell(s) within the gel microdroplet produce an antibody that binds the target microorganism and/or epitope-comprising fragment thereof present in the same gel microdroplet comprises detecting a signal produced by a reporter molecule, wherein the signal is produced in the presence of the modified phenotypic characteristic.

85. The method of claim 84, wherein the microorganism comprises a polynucleotide encoding the reporter molecule.

86. The method of claim 85, wherein the polynucleotide comprises a regulatory region operably linked to a sequence encoding the reporter molecule, wherein the regulatory region is responsive to the modified phenotypic characteristic.

87. The method of claim 86, wherein the regulatory region comprises a promoter.

88. The method of any of claims 82-87, wherein the modified phenotypic characteristic comprises cellular stress and the signal is produced in the presence of the cellular stress.

89. The method of any of claims 83-88, wherein the cellular stress comprises stress to the outer membrane (OM) of the bacterium.

90. The method of any of claims 84-89, wherein the signal produced by the reporter molecule is detected with a detectable moiety.

91. The method of any of claims 84-90, wherein the signal produced by the reporter molecule comprises a fluorescent signal, a luminescent signal, a colorimetric signal, a chemiluminescent signal or a radioactive signal.

92. The method of any of claims 84-91, wherein the reporter molecule is a fluorescent protein, a luminescent protein, a chromoprotein or an enzyme.

93. The method of any of claims 1-78, wherein determining whether the antibody-producing cell(s) within the gel microdroplet produce an antibody that binds the target microorganism and/or epitope-comprising fragment thereof present in the same gel microdroplet comprises determining whether the presence of the antibody kills the target microorganism in the same gel microdroplet, wherein killing of the target microorganism indicates that the antibody specifically binds the target microorganism or epitope-comprising fragment thereof.

94. The method of claim 93, wherein the gel microdroplets comprise a detectable moiety indicative of cell death.

95. The method of any of claims 79-81, 90-92 and 94, wherein the detectable moiety comprises one or more detectable label selected from among a chromophore moiety, a fluorescent moiety, a phosphorescent moiety, a luminescent moiety, a light absorbing moiety, a radioactive moiety, and a transition metal isotope mass tag moiety.

96. The methods of any of claims 1-95, further comprising:
(d) isolating the microdroplet comprising the cell producing the identified antibody or isolating polynucleotides encoding the antibody identified as specifically binding the target microorganism or epitope-comprising fragment thereof.

97. The method of claim 96, wherein isolation is carried out using a micromanipulator or an automated sorter.

98. The method of any of claims 1-97, further comprising:
(e) determining the sequence of the nucleic acids encoding the identified antibody.

99. The method of claim 98, wherein determining the sequence of the nucleic acids is carried out using nucleic acid amplification and/or sequencing.

100. The method of claim 98 or claim 99, wherein determining the sequence of the nucleic acids is carried out using single cell PCR and nucleic acid sequencing.

101. The methods of any of claims 98-100, further comprising:
(f) introducing a polynucleotide comprising a sequence of the nucleic acids encoding the identified antibody or fragment thereof into a cell.

102. The method of any of claims 1-101, wherein the method is completed within about 60 days, 50 days, 40 days, 30 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days or 1 day from completion of step (a).

103. The method of claim 102, wherein the method is completed within about 30 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days or 1 day from completion of step (a).

104. The antibody identified by the method of any of claims 1-103, or an antigen-binding fragment thereof.

105. The antibody or antigen-binding fragment thereof of claim 104, that binds to an epitope present in the at least one conserved region or domain of BamA (β-barrel assembly machinery) of a Gram-negative bacterium.

106. An antibody or antigen-binding fragment thereof, wherein said antibody or antigen-binding fragment thereof binds to an epitope present in at least one conserved region or domain of BamA (β-barrel assembly machinery) of a Gram-negative bacterium.

107. The antibody or antigen-binding fragment thereof of claim 105 or claim 106, wherein the Gram negative bacterium is an Acinetobacter species.

108. The antibody or antigen-binding fragment thereof of any of claim 105-107, wherein the Gram negative bacterium is Acinetobacter baummannii.

109. The antibody or antigen-binding fragment thereof of any of claims 105-108, wherein the conserved region or domain is a conserved region or domain that is shared between BamA from A. baumannii ATCC 19606 and A. baumannii ATCC 17978.

110. The antibody or antigen-binding fragment thereof of claim 109, wherein the conserved region or domain comprises amino acid residues 423-438, 440-460, 462-502, 504-533, 537-544, 547-555, 557-561, 599-604, 606-644, 646-652, 659-700, 702-707, 718-723, 735-747, 749-760, 784-794, 798-804, 806-815 and 817-841 A. baumannii BamA sequence set forth in SEQ ID NO:11.

111. The antibody or antigen-binding fragment thereof of claim 110, wherein the conserved region or domain comprises the sequences set forth in SEQ ID NOS:12-20.

112. The antibody or antigen-binding fragment thereof of any of claims 105-111, wherein the epitope is a contiguous or non-contiguous sequence of the conserved region or domain.

113. The antibody or antigen-binding fragment of any of claims 104-112, wherein the antibody or antigen-binding fragment is human.

114. The antibody or antigen-binding fragment of any of claims 104-112, wherein the antibody or antigen-binding fragment is a humanized antibody.

115. The antibody or antigen-binding fragment of claim 114, wherein the antibody or antigen-binding fragment thereof is produced by antibody-producing cells from a transgenic animal engineered to produce humanized antibodies.

116. The antibody or antigen-binding fragment of any of claims 104-115 wherein the antibody or antigen-binding fragment is recombinant.

117. The antibody or antigen-binding fragment of any of claims 104-116, wherein the antibody or antigen-binding fragment is monoclonal.

118. The antibody or antigen-binding fragment of any of claims 104-117, that is an antigen-binding fragment.

119. The antibody or antigen-binding fragment of any of claims 104-118, wherein said antibody or antigen-binding fragment further comprises an affinity tag, a detectable protein, a protease cleavage sequence, a linker or a nonproteinaceous moiety.

120. The antibody or antigen-binding fragment of any of claims 104-119, wherein:
said antibody or antigen-binding fragment has an equilibrium dissociation constant (K_{D}) for A. baumannii BamA of at or less than or less than about 400 nM, 300 nM, 200 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM.

121. A polynucleotide encoding the antibody or antigen-binding fragment thereof of any of claims 104-120.

122. A composition comprising the antibody of any of claims 104-120.

123. The composition of claim 122, further comprising a pharmaceutically acceptable excipient.

124. A composition comprising a plurality of microdroplets, each microdroplet comprising:
a candidate antibody-producing cell; and
a target microorganism.

125. The composition of claim 124, wherein each microdroplet further comprises the target microorganism or epitope-comprising fragment thereof or a variant thereof bound to a solid support.

126. The composition of claim 124 or claim 125, wherein the target microorganism comprises a polynucleotide encoding a reporter molecule.

127. A library of microdroplets, each microdroplet comprising:
a candidate antibody-producing cell; and
a target microorganism.

128. The library of claim 127, each microdroplet further comprises the target microorganism or epitope-comprising fragment thereof or a variant thereof bound to a solid support.

129. The library of claim 127 or claim 128, wherein the target microorganism comprises a polynucleotide encoding a reporter molecule.


SEQUENCE LISTING (31 sequences)

SEQ ID NO: 1 - protein, 414 aa, Acinetobacter baumannii
  BamA variant 1 (N-terminal deletion)
EEQHSGTTTLAVGYSQSGGITFQAGLSQTNFMGTGNRVAIDLSRSETQDYYNLSVTDPYFTIDGVSRGYNVYYRKTKLNDDYNVNNYVTDSFGGSLSFGYPIDENQSLSASVGVDNTKVTTGAFVSTYVRDYLLANGGKTTSTNTYCLVDLVQDPQTGLYKCPEGQTSQPYGNAFEGEFFTYNLNLGWSYNTLNRPIFPTSGMSHRVGLEIGLPGSDVDYQKVTYDTQAFFPIGSTGFVLRGYGKLGYGNDLPFYKNFYAGGYGSVRGYDNSTLGPKYASVNLQEEKKNDSSPEEVGGNALVQFGTELVLPMPFKGDWTRQVRPVLFAEGGQVFDTKCDVRSYSMIMNGQQISDAKKYCEDNYGFDLGNLRYSVGVGVTWITMIGPLSLSYAFPLNDKPGDETKEIQFEIGRTF

SEQ ID NO: 2 - protein, 816 aa, Acinetobacter baumannii
  BamA variant 2 full length (ATCC 19606)
DDFVVRDIRVNGLVRLTPANVYTMLPINSGDRVNEPMIAEAIRTLYATGLFDDIKASKENDTLVFNVIERPIISKLEFKGNKLIPKEALEQGLKKMGIAEGEVFKKSALQTIETELEQQYTQQGRYDADVTVDTVARPNNRVELKINFNEGTPAKVFDINVIGNTVFKDSEIKQAFAVKESGWASVVTRNDRYAREKMAASLEALRAMYLNKGYINFNINNSQLNISEDKKHIFIEVAVDEGSQFKFGQTKFLGDALYKPEELQALKIYKDGDTYSQEKVNAVKQLLLRKYGNAGYYFADVNIVPQINNETGVVDLNYYVNPGQQVTVRRINFTGNSKTSDEVLRREMRQMEGALASNEKIDLSKVRLERTGFFKTVDIKPARIPNSPDQVDLNVNVEEQHSGTTTLAVGYSQSGGITFQAGLSQTNFMGTGNRVAIDLSRSETQDYYNLSVTDPYFTIDGVSRGYNVYYRKTKLNDDYNVNNYVTDSFGGSLSFGYPIDENQSLSASVGVDNTKVTTGPYVSTYVRDYLLANGGKATSKGTYCPTDANGDSQYDTEKGECKVPEETYDNAFEGEFFTYNLNLGWSYNTLNRPIFPTSGMSHRVGLEIGLPGSDVDYQKVTYDTQAFFPIGSTGFVLRGYGKLGYGNDLPFYKNFYAGGYGSVRGYDNSTLGPKYPSVNLQETKQNDSSPEEVGGNALVQFGTELVLPMPFKGDWTRQVRPVLFAEGGQVFDTKCNIDNSVYGNKGMKINGQTITDVRKYCEDNYGFDLGNLRYSVGVGVTWITMIGPLSLSYAFPLNDKPGDETKEIQFEIGRTF

SEQ ID NO: 3 - protein, 459 aa, Artificial Sequence
  AviTag-10xHis-TEV-A. Baumannii BamA variant 1 (N-terminal deletion)
MSGLNDIFEAQKIEWHEGAHHHHHHHHHHDYDIPTSENLYFQGASEEQHSGTTTLAVGYSQSGGITFQAGLSQTNFMGTGNRVAIDLSRSETQDYYNLSVTDPYFTIDGVSRGYNVYYRKTKLNDDYNVNNYVTDSFGGSLSFGYPIDENQSLSASVGVDNTKVTTGAFVSTYVRDYLLANGGKTTSTNTYCLVDLVQDPQTGLYKCPEGQTSQPYGNAFEGEFFTYNLNLGWSYNTLNRPIFPTSGMSHRVGLEIGLPGSDVDYQKVTYDTQAFFPIGSTGFVLRGYGKLGYGNDLPFYKNFYAGGYGSVRGYDNSTLGPKYASVNLQEEKKNDSSPEEVGGNALVQFGTELVLPMPFKGDWTRQVRPVLFAEGGQVFDTKCDVRSYSMIMNGQQISDAKKYCEDNYGFDLGNLRYSVGVGVTWITMIGPLSLSYAFPLNDKPGDETKEIQFEIGRTF

SEQ ID NO: 4 - protein, 840 aa, Artificial Sequence
  6xHis-A. Baumannii BamA variant 2
MGSSHHHHHHSSGLVPRGSHMASADDFVVRDIRVNGLVRLTPANVYTMLPINSGDRVNEPMIAEAIRTLYATGLFDDIKASKENDTLVFNVIERPIISKLEFKGNKLIPKEALEQGLKKMGIAEGEVFKKSALQTIETELEQQYTQQGRYDADVTVDTVARPNNRVELKINFNEGTPAKVFDINVIGNTVFKDSEIKQAFAVKESGWASVVTRNDRYAREKMAASLEALRAMYLNKGYINFNINNSQLNISEDKKHIFIEVAVDEGSQFKFGQTKFLGDALYKPEELQALKIYKDGDTYSQEKVNAVKQLLLRKYGNAGYYFADVNIVPQINNETGVVDLNYYVNPGQQVTVRRINFTGNSKTSDEVLRREMRQMEGALASNEKIDLSKVRLERTGFFKTVDIKPARIPNSPDQVDLNVNVEEQHSGTTTLAVGYSQSGGITFQAGLSQTNFMGTGNRVAIDLSRSETQDYYNLSVTDPYFTIDGVSRGYNVYYRKTKLNDDYNVNNYVTDSFGGSLSFGYPIDENQSLSASVGVDNTKVTTGPYVSTYVRDYLLANGGKATSKGTYCPTDANGDSQYDTEKGECKVPEETYDNAFEGEFFTYNLNLGWSYNTLNRPIFPTSGMSHRVGLEIGLPGSDVDYQKVTYDTQAFFPIGSTGFVLRGYGKLGYGNDLPFYKNFYAGGYGSVRGYDNSTLGPKYPSVNLQETKQNDSSPEEVGGNALVQFGTELVLPMPFKGDWTRQVRPVLFAEGGQVFDTKCNIDNSVYGNKGMKINGQTITDVRKYCEDNYGFDLGNLRYSVGVGVTWITMIGPLSLSYAFPLNDKPGDETKEIQFEIGRTF

SEQ ID NO: 5 - protein, 419 aa, Acinetobacter baumannii
  BamA variant 3 (N-terminal deletion)
EEQHSGTTTLAVGYSQSGGITFQAGLSQTNFMGTGNRVAIDLSRSETQDYYNLSVTDPYFTIDGVSRGYNVYYRKTKLNDDYNVNNYVTDSFGGSLSFGYPIDENQSLSASVGVDNTKVTTGPYVSTYVRDYLLANGGKATSKGTYCPTDANGDSQYDTEKGECKVPEETYDNAFEGEFFTYNLNLGWSYNTLNRPIFPTSGMSHRVGLEIGLPGSDVDYQKVTYDTQAFFPIGSTGFVLRGYGKLGYGNDLPFYKNFYAGGYGSVRGYDNSTLGPKYPSVNLQETKQNDSSPEEVGGNALVQFGTELVLPMPFKGDWTRQVRPVLFAEGGQVFDTKCNIDNSVYGNKGMKINGQTITDVRKYCEDNYGFDLGNLRYSVGVGVTWITMIGPLSLSYAFPLNDKPGDETKEIQFEIGRTF

SEQ ID NO: 6 - protein, 421 aa, Acinetobacter baumannii
  BamA variant 4 (N-terminal deletion)
EEQHSGTTTLAVGYSQSGGITFQAGLSQTNFMGTGNRVAIDLSRSETQDYYNLSVTDPYFTIDGVSRGYNVYYRKTKLNDDYNVNNYVTDSFGGSLSFGYPIDENQSLSASVGVDNTKVTTGPYVSTYVRDYLLANGGKATGKSSWCPTGKNEVDPKTQQPIPNTCEGGFEPYESAFEGEFFTYNLNLGWSYNTLNRPIFPTSGMSHRVGLEIGLPGSDVDYQKVTYDTQAFFPIGSTGFVIRGYGKLGYGNDLPFYKNFYAGGYGSVRGYDNSTLGPKYASVNLQETKQNDGSPEEVGGNALVQFGTELVLPMPFKGDWTRQVRPVLFAEGGQVFDTKCNIDNTVYGDKGMKINGQTITDVRKYCEDNYGFDLGNLRYSVGVGVTWITMIGPLSLSYAFPLNDKPGDETKEIQFEIGRTF

SEQ ID NO: 7 - protein, 464 aa, Artificial Sequence
  AviTag-10xHis-TEV-A. Baumannii BamA variant 4 (N-terminal deletion)
MSGLNDIFEAQKIEWHEGAHHHHHHHHHHDYDIPTSENLYFQGASEEQHSGTTTLAVGYSQSGGITFQAGLSQTNFMGTGNRVAIDLSRSETQDYYNLSVTDPYFTIDGVSRGYNVYYRKTKLNDDYNVNNYVTDSFGGSLSFGYPIDENQSLSASVGVDNTKVTTGPYVSTYVRDYLLANGGKATSKGTYCPTDANGDSQYDTEKGECKVPEETYDNAFEGEFFTYNLNLGWSYNTLNRPIFPTSGMSHRVGLEIGLPGSDVDYQKVTYDTQAFFPIGSTGFVLRGYGKLGYGNDLPFYKNFYAGGYGSVRGYDNSTLGPKYPSVNLQETKQNDSSPEEVGGNALVQFGTELVLPMPFKGDWTRQVRPVLFAEGGQVFDTKCNIDNSVYGNKGMKINGQTITDVRKYCEDNYGFDLGNLRYSVGVGVTWITMIGPLSLSYAFPLNDKPGDETKEIQFEIGRTF

SEQ ID NO: 8 - protein, 466 aa, Artificial Sequence
  AviTag-10xHis-TEV-A. Baumannii BamA variant 4 (N-terminal deletion)
MSGLNDIFEAQKIEWHEGAHHHHHHHHHHDYDIPTSENLYFQGASEEQHSGTTTLAVGYSQSGGITFQAGLSQTNFMGTGNRVAIDLSRSETQDYYNLSVTDPYFTIDGVSRGYNVYYRKTKLNDDYNVNNYVTDSFGGSLSFGYPIDENQSLSASVGVDNTKVTTGPYVSTYVRDYLLANGGKATGKSSWCPTGKNEVDPKTQQPIPNTCEGGFEPYESAFEGEFFTYNLNLGWSYNTLNRPIFPTSGMSHRVGLEIGLPGSDVDYQKVTYDTQAFFPIGSTGFVIRGYGKLGYGNDLPFYKNFYAGGYGSVRGYDNSTLGPKYASVNLQETKQNDGSPEEVGGNALVQFGTELVLPMPFKGDWTRQVRPVLFAEGGQVFDTKCNIDNTVYGDKGMKINGQTITDVRKYCEDNYGFDLGNLRYSVGVGVTWITMIGPLSLSYAFPLNDKPGDETKEIQFEIGRTF

SEQ ID NO: 9 - protein, 45 aa, Artificial Sequence
  AviTag-10xHis-TEV
MSGLNDIFEAQKIEWHEGAHHHHHHHHHHDYDIPTSENLYFQGAS

SEQ ID NO: 10 - protein, 24 aa, Artificial Sequence
  6xHis
MGSSHHHHHHSSGLVPRGSHMASA

SEQ ID NO: 11 - protein, 841 aa, Acinetobacter baumannii
  A. baumannii ATCC 19606 full length including signal sequence
MRHTHFLMPLALVSAMAAVQQAYAADDFVVRDIRVNGLVRLTPANVYTMLPINSGDRVNEPMIAEAIRTLYATGLFDDIKASKENDTLVFNVIERPIISKLEFKGNKLIPKEALEQGLKKMGIAEGEVFKKSALQTIETELEQQYTQQGRYDADVTVDTVARPNNRVELKINFNEGTPAKVFDINVIGNTVFKDSEIKQAFAVKESGWASVVTRNDRYAREKMAASLEALRAMYLNKGYINFNINNSQLNISEDKKHIFIEVAVDEGSQFKFGQTKFLGDALYKPEELQALKIYKDGDTYSQEKVNAVKQLLLRKYGNAGYYFADVNIVPQINNETGVVDLNYYVNPGQQVTVRRINFTGNSKTSDEVLRREMRQMEGALASNEKIDLSKVRLERTGFFKTVDIKPARIPNSPDQVDLNVNVEEQHSGTTTLAVGYSQSGGITFQAGLSQTNFMGTGNRVAIDLSRSETQDYYNLSVTDPYFTIDGVSRGYNVYYRKTKLNDDYNVNNYVTDSFGGSLSFGYPIDENQSLSASVGVDNTKVTTGPYVSTYVRDYLLANGGKATSKGTYCPTDANGDSQYDTEKGECKVPEETYDNAFEGEFFTYNLNLGWSYNTLNRPIFPTSGMSHRVGLEIGLPGSDVDYQKVTYDTQAFFPIGSTGFVLRGYGKLGYGNDLPFYKNFYAGGYGSVRGYDNSTLGPKYPSVNLQETKQNDSSPEEVGGNALVQFGTELVLPMPFKGDWTRQVRPVLFAEGGQVFDTKCNIDNSVYGNKGMKINGQTITDVRKYCEDNYGFDLGNLRYSVGVGVTWITMIGPLSLSYAFPLNDKPGDETKEIQFEIGRTF

SEQ ID NO: 12 - protein, 16 aa, Acinetobacter baumannii
  A. baumannii ATCC 19606 BamA residues 423-438
EEQHSGTTTLAVGYSQ

SEQ ID NO: 13 - protein, 21 aa, Acinetobacter baumannii
  A. baumannii ATCC 19606 BamA residues 440-460
GGITFQAGLSQTNFMGTGNRV

SEQ ID NO: 14 - protein, 41 aa, Acinetobacter baumannii
  A. baumannii ATCC 19606 BamA residues 462-502
IDLSRSETQDYYNLSVTDPYFTIDGVSRGYNVYYRKTKLND

SEQ ID NO: 15 - protein, 30 aa, Acinetobacter baumannii
  A. baumannii ATCC 19606 BamA residues 504-533
YNVNNYVTDSFGGSLSFGYPIDENQSLSAS

SEQ ID NO: 16 - protein, 8 aa, Acinetobacter baumannii
  A. baumannii ATCC 19606 BamA residues 537-544
DNTKVTTG

SEQ ID NO: 17 - protein, 9 aa, Acinetobacter baumannii
  A. baumannii ATCC 19606 BamA residues 547-555
VSTYVRDYL

SEQ ID NO: 18 - protein, 5 aa, Acinetobacter baumannii
  A. baumannii ATCC 19606 BamA residues 557-561
ANGGK

SEQ ID NO: 19 - protein, 6 aa, Acinetobacter baumannii
  A. baumannii ATCC 19606 BamA residues 599-604
GEFFTY

SEQ ID NO: 20 - protein, 39 aa, Acinetobacter baumannii
  A. baumannii ATCC 19606 BamA residues 606-644
LNLGWSYNTLNRPIFPTSGMSHRVGLEIGLPGSDVDYQK

SEQ ID NO: 21 - protein, 7 aa, Acinetobacter baumannii
  A. baumannii ATCC 19606 BamA residues 646-652
TYDTQAF

SEQ ID NO: 22 - protein, 42 aa, Acinetobacter baumannii
  A. baumannii ATCC 19606 BamA residues 659-700
GFVLRGYGKLGYGNDLPFYKNFYAGGYGSVRGYDNSTLGPKY

SEQ ID NO: 23 - protein, 6 aa, Acinetobacter baumannii
  A. baumannii ATCC 19606 BamA residues 702-707
SVNLQE

SEQ ID NO: 24 - protein, 6 aa, Acinetobacter baumannii
  A. baumannii ATCC 19606 BamA residues 718-723
VGGNAL

SEQ ID NO: 25 - protein, 13 aa, Acinetobacter baumannii
  A. baumannii ATCC 19606 BamA residues 735-747
PFKGDWTRQVRPV

SEQ ID NO: 26 - protein, 12 aa, Acinetobacter baumannii
  A. baumannii ATCC 19606 BamA residues 749-760
FAEGGQVFDTKC

SEQ ID NO: 27 - protein, 11 aa, Acinetobacter baumannii
  A. baumannii ATCC 19606 BamA residues 784-794
KYCEDNYGFDL

SEQ ID NO: 28 - protein, 7 aa, Acinetobacter baumannii
  A. baumannii ATCC 19606 BamA residues 798-804
RYSVGVG

SEQ ID NO: 29 - protein, 10 aa, Acinetobacter baumannii
  A. baumannii ATCC 19606 BamA residues 806-815
TWITMIGPLS

SEQ ID NO: 30 - protein, 25 aa, Acinetobacter baumannii
  A. baumannii ATCC 19606 BamA residues 817-841
SYAFPLNDKPGDETKEIQFEIGRTF

SEQ ID NO: 31 - protein, 414 aa, Artificial Sequence
  BamA variant 5 (N-terminal deletion)
EEQHSGTTTLAVGYSQSGGITFQAGLSQTNFMGTGNRVAIDLSRSETQDYYNLSVTDPYFTIDGVSRGYNVYYRKTKLNDDYNVNNYVTDSFGGSLSFGYPIDENQSLSASVGVDNTKVTTGPYVSTYVRDYLLANGGKATSKGTYCPTDANGDSQYDTEKGECKVPEETYDNAFEGEFFTYNLNLGWSYNTLNRPIFPTSGMSHRVGLEIGLPGSDVDYQKVTYDTQAFFPIGSTGFVLRGYGKLGYGNDLPFYKNFYAGGYGSVRGYDNSTLGPKYASVNLQEEKKNDSSPEEVGGNALVQFGTELVLPMPFKGDWTRQVRPVLFAEGGQVFDTKCDVRSYSMIMNGQQISDAKKYCEDNYGFDLGNLRYSVGVGVTWITMIGPLSLSYAFPLNDKPGDETKEIQFEIGRTF